SEQUENCE LISTING (15 sequences)

SEQ ID NO: 1 - DNA, 7696 bp, Artificial Sequence
  synthetic sequence   human fumarylacetoacetate hydrolase as inserted into a lentiviral vector
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatagcgctacgcgtcctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcgcgccaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtggctaagcttggtaccgagctcggatccgcttagcatgtccttcatcccggtggccgaggattccgacttccccatccacaacctgccctacggcgtcttctcgaccagaggcgacccaagaccgaggataggtgtggccattggcgaccagatcctggacctcagcatcatcaagcacctctttactggtcctgtcctctccaaacaccaggatgtcttcaatcagcctacactcaacagcttcatgggcctgggtcaggctgcctggaaggaggcgagagtgttcttgcagaacttgctgtctgtgagccaagccaggctcagagatgacaccgaacttcggaagtgtgcattcatctcccaggcttctgccacgatgcaccttccagccaccataggagactacacagacttctattcctctcggcagcatgctaccaacgtcggaatcatgttcagggacaaggagaatgcgttgatgccaaattggctgcacttaccagtgggctaccatggccgtgcctcctctgtcgtggtgtctggcaccccaatccgaaggcccatgggacagatgaaacctgatgactctaagcctcccgtatatggtgcctgcaagctcttggacatggagctggaaatggctttttttgtaggccctggaaacagattgggagagccgatccccatttccaaggcccatgagcacatttttggaatggtccttatgaacgactggagtgcacgagacattcagaagtgggagtatgtccctctcgggccattccttgggaagagttttgggaccactgtctctccgtgggtggtgcccatggatgctctcatgccctttgctgtgcccaacccgaagcaggaccccaggcccctgccgtatctgtgccatgacgagccctacacatttgacatcaacctctctgttaacctgaaaggagaaggaatgagccaggcggctaccatatgcaagtccaattttaagtacatgtactggacgatgctgcagcagctcactcaccactctgtcaacggctgcaacctgcggccgggggacctcctggcttctgggaccatcagcgggccggagccagaaaacttcggctccatgttggaactgtcgtggaagggaacgaagcccatagacctggggaatggtcagaccaggaagtttctgctggacggggatgaagtcatcataacagggtactgccagggggatggttaccgcatcggctttggccagtgtgctggaaaagtgctgcctgctctcctgccatcatgagaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtattataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 2 - DNA, 29 bp, Artificial Sequence
  Forward primer
tgtgtgactctggtaactagagatccctc

SEQ ID NO: 3 - DNA, 20 bp, Artificial Sequence
  Reverse primer
agtagcacccaccaaggcaa

SEQ ID NO: 4 - DNA, 23 bp, Artificial Sequence
  Forward primer
ggatgtcttcaatcagcctacac

SEQ ID NO: 5 - DNA, 22 bp, Artificial Sequence
  Reverse primer
tgaaacctgatgactctaagcc

SEQ ID NO: 6 - DNA, 20 bp, Artificial Sequence
  Forward primer
tgttggaactgtcgtggaag

SEQ ID NO: 7 - DNA, 20 bp, Artificial Sequence
  Reverse primer
taactagggaacccactgct

SEQ ID NO: 8 - DNA, 1260 bp, Artificial Sequence
  Nucleic acid sequence for human fumarylacetoacetate hydrolase
atgtccttcatcccggtggccgaggattccgacttccccatccacaacctgccctacggcgtcttctcgaccagaggcgacccaagaccgaggataggtgtggccattggcgaccagatcctggacctcagcatcatcaagcacctctttactggtcctgtcctctccaaacaccaggatgtcttcaatcagcctacactcaacagcttcatgggcctgggtcaggctgcctggaaggaggcgagagtgttcttgcagaacttgctgtctgtgagccaagccaggctcagagatgacaccgaacttcggaagtgtgcattcatctcccaggcttctgccacgatgcaccttccagccaccataggagactacacagacttctattcctctcggcagcatgctaccaacgtcggaatcatgttcagggacaaggagaatgcgttgatgccaaattggctgcacttaccagtgggctaccatggccgtgcctcctctgtcgtggtgtctggcaccccaatccgaaggcccatgggacagatgaaacctgatgactctaagcctcccgtatatggtgcctgcaagctcttggacatggagctggaaatggctttttttgtaggccctggaaacagattgggagagccgatccccatttccaaggcccatgagcacatttttggaatggtccttatgaacgactggagtgcacgagacattcagaagtgggagtatgtccctctcgggccattccttgggaagagttttgggaccactgtctctccgtgggtggtgcccatggatgctctcatgccctttgctgtgcccaacccgaagcaggaccccaggcccctgccgtatctgtgccatgacgagccctacacatttgacatcaacctctctgttaacctgaaaggagaaggaatgagccaggcggctaccatatgcaagtccaattttaagtacatgtactggacgatgctgcagcagctcactcaccactctgtcaacggctgcaacctgcggccgggggacctcctggcttctgggaccatcagcgggccggagccagaaaacttcggctccatgttggaactgtcgtggaagggaacgaagcccatagacctggggaatggtcagaccaggaagtttctgctggacggggatgaagtcatcataacagggtactgccagggggatggttaccgcatcggctttggccagtgtgctggaaaagtgctgcctgctctcctgccatcatga

SEQ ID NO: 9 - protein, 419 aa, Artificial Sequence
  Protein sequence for human fumarylacetoacetate hydrolase
MSFIPVAEDSDFPIHNLPYGVFSTRGDPRPRIGVAIGDQILDLSIIKHLFTGPVLSKHQDVFNQPTLNSFMGLGQAAWKEARVFLQNLLSVSQARLRDDTELRKCAFISQASATMHLPATIGDYTDFYSSRQHATNVGIMFRDKENALMPNWLHLPVGYHGRASSVVVSGTPIRRPMGQMKPDDSKPPVYGACKLLDMELEMAFFVGPGNRLGEPIPISKAHEHIFGMVLMNDWSARDIQKWEYVPLGPFLGKSFGTTVSPWVVPMDALMPFAVPNPKQDPRPLPYLCHDEPYTFDINLSVNLKGEGMSQAATICKSNFKYMYWTMLQQLTHHSVNGCNLRPGDLLASGTISGPEPENFGSMLELSWKGTKPIDLGNGQTRKFLLDGDEVIITGYCQGDGYRIGFGQCAGKVLPALLPS

SEQ ID NO: 10 - DNA, 28 bp, Artificial Sequence
  Synthetic sequences primers for identifying sites of integration (FIG.1)
atgtgactctggtaactagagatccctc

SEQ ID NO: 11 - DNA, 26 bp, Artificial Sequence
  Synthetic sequences primers for identifying sites of integration (FIG.1)
gagatccctcagacccttttagtcag

SEQ ID NO: 12 - DNA, 17 bp, Artificial Sequence
  MseI linker
tagtcccttaagcggag

SEQ ID NO: 13 - DNA, 37 bp, Artificial Sequence
  MseI linker
gtaatacgactcactatagggcctccgcttaagggac

SEQ ID NO: 14 - DNA, 83 bp, Artificial Sequence
  Linker primer (reverse primer)
caagcagaagacggcatacgagatcggtctcggcattcctgctgaaccgctcttccgatctgtaatacgactcactatagggc

SEQ ID NO: 15 - DNA, 90 bp, Artificial Sequence
  Nested LSP
aatgatacggcgaccaccgagatctacactctttccctacacgacgctcttccgatctcgatgtgagatccctcagacccttttagtcag

(19) United States
(12) Patent Application Publication
(10) Pub. No.: US 2020/0216823 A1
(43) Pub. Date: Jul. 9, 2020

(54) VECTORS AND METHODS OF USE

(71) Applicant: Children's Hospitals and Clinics of Minnesota, Minneapolis, MN (US)

(72) Inventor: Joseph LILLEGARD, Minneapolis, MN (US)

(73) Assignee: Children's Hospitals and Clinics of Minnesota, Minneapolis, MN (US)

(21) Appl. No.: 16/775,918

(22) Filed: Jan. 29, 2020

Related U.S. Application Data
(63) Continuation of application No. PCT/US2018/054219, filed on Oct. 3, 2018.
(60) Provisional application No. 62/567,631, filed on Oct. 3, 2017.
(60) Provisional application No. 62/588,106, filed on Nov. 17, 2017.
(60) Provisional application No. 62/640,787, filed on Mar. 9, 2018.

(51) Int. Cl. C12N 9/02; C12N 15/86; C12N 9/14; C12N 7/00; A61K 9/00; A61K 38/46; C12N 5/071

(52) U.S. Cl. CPC C12N 9/0071; C12N 15/86; C12N 9/14; C12Y 114/16001; C12N 7/00; C12N 2510/00; A61K 38/46; C12Y 307/01002; C12N 5/067; C12N 2740/15043; A61K 9/0019

(57) ABSTRACT

This disclosure provides vectors and strategies for increasing the efficiency of gene therapy in hepatocytes. The efficiency of the delivery of a corrected gene or wild type gene is improved through the use of delivery to hepatocytes via intrahepatic (parenchyma) administration or administration via the portal vein. In addition, the corrected gene or wild type gene is delivered using an isolated exogenous nucleic acid comprising a promoter that is specifically expressed in hepatocytes. These methods and isolated exogenous nucleic acids are useful to correct gene defects in the liver such as inherited diseases of the liver.

### CROSS-REFERENCE TO RELATED APPLICATIONS

This application is a Continuation of PCT International patent application No. PCT/US2018/054219, filed Oct. 3, 2018, which claims priority to U.S. Provisional Patent Application No. 62/567,631, filed Oct. 3, 2017, U.S. Provisional Patent Application No. 62/588,106, filed Nov. 17, 2017, and to U.S. Provisional Patent Application No. 62/640,787, filed Mar. 9, 2018, the disclosures of which are hereby incorporated by reference herein in their entirety.

### SEQUENCE LISTING SUBMISSION

The present application includes a Sequence Listing in electronic format as a txt file titled “SEQUENCE-LISTING,” which was originally created on Oct. 2, 2018 and revised on Jan. 27, 2020, which has a size of 18.2 kilobytes (KB). The contents of txt file “SEQUENCE-LISTING” are incorporated by reference herein.

### BACKGROUND

Gene therapy is useful to cure many diseases by repairing or replacing a defective gene responsible for disease. Traditional gene therapies have been best suited to treat diseases of deficient or absent gene products rather than those diseases caused by aberrantly functioning proteins. Even now, gene therapy efforts remain focused on gene addition strategies using vector-borne full-length complementary DNA cassettes for the mutated gene of interest (Mueller C, Flotte T R. Clinical gene therapy using recombinant adeno-associated virus vectors. Gene Ther. 2008; 15:858-863.) Vectors useful in this process include adeno associated virus, lentiviral vectors, or retroviral vectors. Despite many advances, gene addition approaches with adeno-associated virus (AAV) are limited by transient and unregulated expression, infrequent highly random integrations, transgene silencing and increased mutagenic and oncogenic risks. Not all protein-coding genes have open reading frames small enough to fit within the low coding capacity of AAV (4.7 kb), thus, this type of gene therapy is not applicable for all disorders.

Gene repair strategies using homologous site-specific recombination to repair the underlying mutation are developing. Contrary to more widely used gene addition paradigms, gene repair restores gene function within the context of all endogenous regulatory elements, thereby eliminating potential problems with inadequate or inappropriate expression. Different constructs have been utilized for performing gene repair including single-strand oligonucleotides, triplex-forming oligonucleotides, RNA-DNA hybrids, small fragment DNA, and AAV. However, these strategies have suffered from very low rates of site-specific recombination and as a result have not been viewed as therapeutically effective.

Thus there is a need to identify vectors and targeting strategies for more efficient delivery and integration of genetic material at a specific site and in a specific tissue in order to replace or repair genetic defects responsible for disease.

### SUMMARY

This disclosure provides vectors and strategies for increasing the efficiency of gene therapy in hepatocytes. The efficiency of the delivery of a corrected gene or wild type gene is improved through the use of delivery to hepatocytes via intrahepatocyte administration or administration via the portal vein. In addition, the corrected gene or wild type gene is delivered using an isolated exogenous nucleic acid comprising a promoter that is specifically expressed in hepatocytes. These methods and isolated exogenous nucleic acids are useful to correct gene defects in the liver such as inherited diseases of the liver, including hereditary tyrosinemias (HTI), phenyl ketonuria (PKU), Wilson's disease, alpha-1-antitrypsin deficiency, and urea cycle disorder.

In a first aspect, the disclosure provides a method of delivering an isolated exogenous nucleic acid coding for all or part of a polypeptide to the liver comprising administering the isolated exogenous nucleic acid to hepatocytes of the subject, such as a fetus, infant, or older pediatric patient, by delivering the isolated exogenous nucleic acid intrahepatically or via the portal vein. In embodiments, the polypeptide is a functional polypeptide that replaces the defective or absent polypeptide due to a genetic defect in the subject. In embodiments, the isolated exogenous nucleic acid comprises a vector. In embodiments, the vector provides for integration into the genome of the hepatocyte. In some embodiments, the isolated exogenous nucleic acid integrates at the location of a nucleic acid coding for all or part of the polypeptide in the genome of the hepatocyte. In embodiments, the nucleic acid coding for all or part of the polypeptide in the genome of the hepatocyte codes for a polypeptide that has decreased function in the hepatocytes. In embodiments, the nucleic acid coding for all or part of the polypeptide in the isolated nucleic acid codes for a polypeptide having increased function as compared to that in the hepatocytes. In embodiments, the isolated nucleic acid integrates more often at the location of a nucleic acid coding for all or part of the polypeptide in the genome of the hepatocyte.

In other embodiments, the vector should be entirely consumed by the liver, precluding exposure and subsequent integration in organs other than the liver. Non limiting examples of such a vector include a lentivirus, an adeno associated virus, and a retrovirus. When a vector such as a lentivirus is employed, the vector is serotyped and the dose is selected to minimize dissemination to other tissues, such as a dose of approximately 10¹⁰ TU/kg or less. In embodiments, the lentiviral vector is a self-inactivating vector. In other embodiments the lentiviral vector expresses a glycoprotein that helps to restrict the viral vector to liver cells. In some embodiments, the glycoprotein is derived from vesicular stomatitis virus (VSV). In embodiments, the vector shows minimal preference for integrating in or near tumorigenesis genes. In embodiments, a vector is selected that shows a preference for integration based on sequence homology to a nucleic acid coding for all or part of the polypeptide in the genome of the hepatocyte. In embodiments, the vector has decreased integration into coding regions for tumor related genes, The term “decreased integration” as used herein means as compared to the sites of homology with the polypeptide and/or the promoter region in the vector, the number of integrations of the vector into tumorigenic genes is at least 2 fold less, at least 4 fold less, at least 10 fold less, at least 100 fold or less than that at sites of homology between the vector and the genome.

In embodiments, the isolated exogenous nucleic acid comprises double stranded oligonucleotides, single-strand oligonucleotides, triplex-forming oligonucleotides, RNA-DNA hybrids, or a small fragment DNA. In certain embodiments, the double stranded oligonucleotides, single-strand oligonucleotides, triplex-forming oligonucleotides, RNA-DNA hybrids, or a small fragment DNA code for all or a part of a polypeptide. In certain embodiments, the double stranded oligonucleotides, single-strand oligonucleotides, triplex-forming oligonucleotides, RNA-DNA hybrids, or a small fragment DNA code for a corrected sequence for a mutation in an intron, such as a mutation that results in a truncation or deletion of one or more exons. In some embodiments, double stranded oligonucleotides, single-strand oligonucleotides, triplex-forming oligonucleotides, RNA-DNA hybrids, or a small fragment DNA are included in a vector, such as a plasmid or the viral vectors described herein. When a vector such as a lentivirus is employed, the vector is serotyped and the dose is selected to minimize dissemination to other tissues, such as a dose of approximately 10¹⁰ TU/kg or less

In embodiments, the isolated exogenous nucleic acid codes for all or part of a wild type polypeptide or a functional polypeptide. In other embodiments, all or part of the polypeptide comprises a functional fumarylacetoacetate hydrolase, a functional tyrosine transaminase, a functional 4-hydroxy-phenylpyruvate dioxygenase, or a functional phenylalanine hydroxylase. In embodiments, the isolated exogenous nucleic acid comprises a lentiviral vector, wherein the lentiviral vector comprises a spleen focus forming virus promoter, a cytomegalovirus promoter, or an alpha1-antitrypsin (AAT) promoter operably linked to all or part of the isolated exogenous nucleic acid coding for all or part of the polypeptide.

In certain embodiments, promoters are selected to provide for gene expression in hepatocytes including alpha 1 anti trypsin promoter or thyroxin binding globulin promoter. In embodiments, the promoter is the alpha 1 antitrypsin promoter. In embodiments, a promoter can be an inducible promoter. In embodiments, a promoter is selected that has low genotoxicity. Standard in vitro genotoxicity assays include the bacterial reverse mutation assay, the in vitro mammalian chromosomal aberration test, the in vitro mammalian cell gene mutation test [Hprt] and [MLA/tk]), and the in vitro mammalian cell micronucleus test. Confirmatory in vivo tests are also known. (Corvi et al., Food and Chemical Toxicity, 2017, 106:600.) In embodiments, a low genotoxic promoter is the alpha1 antitrypsin promoter. In embodiments, a promoter is selected that has decreased integration in tumorigenesis genes as compared to the chicken βactin promoter.

The promoter region further comprises enhancer regions that are specific to hepatocytes such as those associated with the alpha 1 anti trypsin promoter or the hepatic control region enhancer. In addition, a hepatocyte specific promoter is selected that minimizes any expression in non-target cells (non-hepatocytes). In embodiments, the promoter/enhancer region is minimized in order to maximize the space in the vector for transgenes. In embodiments, the promoter enhancer region is 1000 bp or less, 900 bp or less, 800 bp or less, 700 bp or less, 600 bp or less, 500 bp or less, 400 bp or less, or 300 bp or less.

In another aspect of the disclosure, the isolated exogenous nucleic acid is contained with a hepatocyte from the same species as the subject. In embodiments, the hepatocyte is obtained from or derived from the fetus, infant, or other pediatric patient to be treated. The hepatocytes are expanded ex vivo and transduced, transfected, or transformed with the isolated exogenous nucleic acid that provides for expression of all or a part of a polypeptide. In embodiments, the polypeptide is one that has a decreased function in the hepatocytes due to a genetic alteration.

In embodiments, the expansion of the hepatocytes can be conducted in a genetically altered animal that is immunosuppressed such as a severe combined immunodeficient (SCID) pig homozygous for a disruption in the fumarylacetoacetate hydrolase (Fah) gene such that the disruption results in loss of expression of functional FAH protein, wherein the pig exhibits decreased liver function. These pigs are referred to as fumarylacetoacetate hydrolase (FAH)-deficient pigs or FAH −/−. In embodiments the FAH−/− pig is a fetal pig and the hepatocytes are expanded in a fetal liver in utero. Production of such pigs and methods of expanding hepatocytes in them are described in U.S. Pat. Nos. 9,000,257 and 9,485,971, which are hereby incorporated by reference In embodiments, one or more corrected genes or wild type genes in the isolated exogenous nucleic acid sequence in the hepatocytes can be expanded in the FAH−/− pig including those that further comprise a nucleic acid coding for a functional fumarylacetoacetate hydrolase as these cells have a competitive advantage as compared to FAH−/− hepatocytes, such as those endogenous to the FAH−/− pig. In embodiments, the isolated exogenous nucleic acid comprises a nucleic acid coding for a functional fumarylacetoacetate hydrolase and a nucleic acid coding for a functional polypeptide comprising phenylalanine hydroxylase, a tyrosine transaminase, a 4-hydroxy-phenylpyruvate dioxygenase or combinations thereof.

In addition, the FAH−/− pig can be immunosuppressed either by knocking out genes or by treating the animal with an immunosuppressive agent. Any suitable immunosuppressive agent or agents effective for achieving immunosuppression in a pig can be used. Examples of immunosuppressive agents include, but are not limited to, FK506, cyclosporin A, fludarabine, mycophenolate, prednisone, rapamycin and azathioprine. Combinations of immunosuppressive agents can also be administered. In some embodiments, the one or more immunosuppressive agents are administered to the Fah-deficient pig at least about 2 days prior to human hepatocyte transplantation. Immunosuppression may be continued for a period of time, for example for a portion of or the entire life of the pig. Although the use of immunosuppressive drugs is exemplified for obtaining immunodeficient animals, also contemplated are genetic alterations that result in a lack of a specific component of the immune system, or a lack of functionality of a specific component of the immune system (such as a lack of functional B, T and/or NK cells). In some embodiments, the genetic alteration is in the Rag1, Rag2 and/or Il2rg gene. Specific cells of the immune system (such as macrophages or NK cells) can also be depleted. Methods of depleting particular cell types are known in the art. Moreover, the two disclosed methods of expanding human hepatocytes in Fah-deficient pigs can be combined. Fetal transplantation can be carried out to induce tolerance to the human hepatocytes, thus permitting the administration of a larger dose of human hepatocytes postnatally.

In embodiments, a method comprises expanding human hepatocytes in vivo by transplanting human hepatocytes into an immunodeficient FAH−/− pig and allowing the human hepatocytes to expand. In embodiments, the hepatocytes are delivered via the portal vein, intrahepatically, or to lymph nodes. In embodiments, the FAH−/− pig also lacks a functional gene for Rag1, Rag2, Il2rg gene, or combinations thereof. In embodiments, the human hepatocyte is obtained from or derived from the fetus, the infant, or older pediatric patient and as such, are considered autologous to the fetus or infant. The hepatocytes are transduced, transfected, or transformed with the isolated exogenous nucleic acid that provides for expression of a functional polypeptide, that is defective in the cells of the fetus, infant, or older pediatric patient.

In embodiments, the isolated nucleic acid introduced into a hepatocyte derived from the subject can have a further alteration in order to provide a selective advantage to those transduced cells. In embodiments, the FAH gene is added to the isolated nucleic acid in combination with the transgene that corrects another metabolic defect such as PKU. Other gene alterations in hepatocytes or the isolated nucleic acid providing selective advantage include overexpression of Forkhead boxM1 transcriptional activator or expression of Bcl-2.

The length of time for hepatocyte expansion can vary and will depend on a variety of factors, including the number of hepatocytes originally transplanted, the number of human hepatocytes desired following expansion and/or the desired degree of liver repopulation with the human hepatocytes. In some cases, these factors will be dictated by the desired use of the hepatocytes or the desired use of the Fah-deficient pig engrafted with the human hepatocytes. In some embodiments, the human hepatocytes are expanded in the Fah-deficient pig of Fah-deficient fetal pig for at least about 3 days, at least about 5 days, at least about 7 days, at least about 2 weeks, or at least about 4 weeks, at least about 2 months, at least about 3 months, at least about 4 months, at least about 5 months, at least about 6 months, at least about 7 months, at least about 8 months, at least about 9 months, at least about 10 months or at least about 11 months. In particular examples, the human hepatocytes are expanded in the Fah-deficient pig for at least 7 days. In other examples, the human hepatocytes are expanded in the Fah-deficient pig for at least 6 months. In some examples, the human hepatocytes are expanded in the Fah-deficient pig no more than 12 months.

In some embodiments, the FAH−/−-deficient pig is administered an agent that inhibits, delays or prevents the development of liver disease in the pig. Administration of such an agent is necessary to prevent liver dysfunction and/or death of the animal prior to repopulation of the animal with healthy (FAH-expressing) hepatocytes. The agent can be any compound or composition known in the art to inhibit liver disease. One such agent is 2-(2-nitro-4-trifluoro-methyl-benzoyl)-1, 3 cyclohexanedione (NTBC), but other pharmacologic inhibitors of phenylpyruvate dioxygenase, such as methyl-NTBC can be used. NTBC (or a similar compound) is administered to regulate the development of liver disease in the Fah-deficient pig. The dose, dosing schedule and method of administration can be adjusted as needed to prevent liver dysfunction in the Fah-deficient pig or Fah-deficient fetal pig. In some embodiments, the FAH−/−-deficient pig is further administered NTBC for at least two days, at least three days, at least four days, at least five days or at least six days following hepatocyte transplantation. In some embodiments, the FAH−/− deficient pig is further administered NTBC for at least about one week, at least about two weeks, at least about three weeks, at least about four weeks, at least about one month, at least about two months, at least about three months, at least about four months, at least about five months, or at least about six months.

In embodiments, once expanded, the hepatocytes are collected from the animal and delivered to a fetus, infant, or older pediatric patient. In embodiments, the fetus, infant, or older pediatric patient have been screened for a genetic defect, and the isolated nucleic acid and/or hepatocyte containing the isolated nucleic acid codes for a nucleic acid that can correct the genetic defect or provide for a polypeptide with increased or otherwise redundant/therapeutic function as compared to the polypeptide that is defective.

In embodiments, the hepatocytes are delivered intrahepatically, via the portal vein, or delivered to lymph nodes. In embodiments, during delivery via the portal vein, the delivery is ultrasound guided percutaneous delivery. In further embodiments, the portal pressure is monitored during delivery and stopped or paused if the portal pressure is 2 mm Hg, 3 mm Hg, 4 mm Hg, 5 mm Hg, 6 mm Hg, 7 mm Hg, or 8 mm Hg or greater than normal portal pressures. Normal portal pressures in infants or children are about 5 to 10 mm Hg. In embodiments, the subject can also be treated with an anticoagulant before, during, and/or after administration.

In embodiments, the transduced or transformed hepatocytes can be delivered as single cells or in spheroids. In embodiments, about 4 to 15 grams of single cell suspension is delivered to the animals. In other embodiments, about 9 to 12 grams of spheroids are delivered to the animals with or without a pharmaceutical agent to prevent thrombosis, such as enoxaparin. In certain embodiments, at least about 1×10⁹ to 1×10¹² cells are delivered.

In yet other embodiments, the isolated exogenous nucleic acid is contained within a stem cell. In embodiments, the stem cell is an adult stem cell, a hepatic stem cell, a fetal stem cell, or an embryonic stem cell. In embodiments, the stem cell is obtained from or derived from the same species of the subject. In embodiments the stem cell is obtained from or derived from the same fetus, infant, or older pediatric patient.

In certain embodiments, the subject is a human. In embodiments, the human is a fetus, neonate, infant or older pediatric patient. In embodiments, the fetus is at least 4 weeks old, 5 weeks old, 6 weeks old, 7 weeks old, 8 weeks old, 9 weeks old, 10 weeks old, 11 weeks old, 12 weeks old, 13 weeks old, 14 weeks old, 15 weeks old, 16 weeks old, 17 weeks old, 18 weeks old, 19 weeks old, 20 weeks old, 21 weeks old, 22 weeks old, 23 weeks old, 24 weeks old, 25 weeks old, 26 weeks old, 27 weeks old, 28 weeks old, 29 weeks old, 30 weeks old, 31 weeks old, 32 weeks old, 33 weeks old, 34 weeks old, 35 weeks old, 36 weeks old, 37 weeks old, 38 weeks old, 39 weeks old, or 40 weeks old. In embodiments the human is a neonate that is at least 1 day old, 2 days old, 3 days old, 4 days old, 5 days old, 6 days old, 7 days old, 8 days old, 9 days old, 10 days old, 11 days old, 12 days old, 13 days old, 14 days old, 15 days old, 16 days old, 17 days old, 18 days old, 19 days old, 20 days old, 21 days old, 22 days old, 23 days old, 24 days old, 25 days old, 26 days old, 27 days old, or 28 days old. In certain embodiments, the human is an infant at least 29 days old. In other embodiments, the infant is 1 year old or less. In embodiments, the human is an older pediatric patient at least 1 year old or greater, and/or 18 years old or less.

In embodiments, a method for treating hereditary tyrosinemia comprises administering an isolated exogenous nucleic acid to hepatocytes of a subject, such as a fetus, infant, or older pediatric patient by delivering the isolated exogenous nucleic acid intrahepatically or via the portal vein, wherein the isolated exogenous nucleic acid codes for all or part of a functional fumarylacetoacetate hydrolase, a functional tyrosine transaminase, a functional p hydroxyphenyl pyruvate dioxegenase, or combinations thereof, under control of a hepatocyte specific promoter. In some embodiments, the isolated exogenous nucleic acid is delivered to lymph nodes. In embodiments, the isolated exogenous nucleic acid comprises a vector. In embodiments, the hepatocyte specific promoter is an alpha1 anti trypsin (also referred to SERPINA1) promoter. In embodiments, any of the isolated exogenous nucleic acids as described herein can be utilized in the method of treating.

In embodiments, a method for treating phenyl ketonuria comprises administering an isolated exogenous nucleic acid to hepatocytes of a subject, such as a fetus, infant, or older pediatric patient by delivering the isolated exogenous nucleic acid intrahepatically or via the portal vein, wherein the isolated exogenous nucleic acid codes for all or part of a functional phenylalanine hydroxylase under control of a hepatocyte specific promoter. In some embodiments, the isolated exogenous nucleic acid is delivered to lymph nodes. In embodiments, the isolated exogenous nucleic acid comprises a vector. In embodiments, the hepatocyte specific promoter is an alpha1 anti trypsin promoter. In embodiments, any of the isolated exogenous nucleic acids as described herein can be utilized in the method of treating.

In some cases, genetic defects can be due to many different mutations in a gene. In such cases, a biological sample is isolated and analyzed for the specific mutation in the gene of interest. Once the mutation is identified, an isolated exogenous nucleic acid can be designed to replace or repair the specific mutation. In addition treated subjects can be monitored for correction of any metabolic defect such as by measuring the levels of liver compounds or other biomarkers such as AST, alkaline phosphatase, tyrosine, ammonia, total bilirubin, and/or international normalized ratio (INR). In addition, markers of fibrosis or cirrhosis can be monitored, for example, by imaging. Treatment can be continued until the level of these markers return to normal values for the subject.

In another aspect of the disclosure, a vector for providing a functional polypeptide to a hepatocyte is disclosed. In embodiments, the vector is a lentiviral vector comprising an isolated exogenous nucleic acid coding for all or part of a polypeptide operably linked to a hepatocyte specific promoter. In embodiments, all or part of the polypeptide codes for a functional fumarylacetoacetate hydrolase, a functional tyrosine transaminase, a functional 4-hydroxyphenyl pyruvate dioxegenase, a functional phenylalanine hydroxylase. In embodiments, the hepatocyte specific promoter is an alpha 1 anti trypsin promoter. In embodiments, the hepatocyte specific promoter also comprises an enhancer.

### BRIEF DESCRIPTION OF THE DRAWINGS

FIG. 1 is a schematic diagram showing the arrangement of elements in a lentiviral vector construct. The lentiviral backbone contains the 5′ long terminal repeat (LTR) followed by the psi packaging sequence (Ψ) followed by the rev responsive element (RRE), the central polypurine tract (cPPT), the woodchuck hepatitis virus posttranscriptional regulatory element (WPRE), and the 3′ LTR with all or part of the U3 region deleted. A nucleic acid comprising a hepatic control region enhancer (HCR) operably linked to the human alpha 1 anti trypsin promoter (hAAT)(SerpinAl exon 1; 252 bp) operably linked to the human FAH gene (herein “GENE”) was inserted into the Lentiviral backbone between the cPPT site and the WPRE site using ClaI and BamHI restriction sites for the AAT promoter, followed by newly introduced BlpI and EcoRI for the FAH gene. Primers are shown for identifying sites of integration of the vector.

FIG. 2A shows luciferase expression in the abdomens of wild type mouse fetuses injected intrahepatically in utero with a lentiviral vector carrying the luciferase gene under control of the cytomegalovirus (CMV) promoter. Injections were performed at day 15 of gestation with 3×10⁸ transducing units (TU)/fetus. Mothers and fetuses that received luciferase were imaged with the Xenogen IVIS-200 system 3-5 days after injection. FIG. 2B shows distribution of expression limited to liver (top right) and absent in the hepatectomied fetus (left) and other abdominal organs (bottom right) resulting from this procedure, indicating that this is a potentially valuable route of administration for treating these diseases in the developing fetus without exposing off-target organs.

FIGS. 3A to 3E show biodistribution of single cells or spheroids of hepatocytes transduced with lentiviral construct ex vivo in pigs and mice. Wild-type pigs received equal doses of Zirconium 89-labeled cells and spheroids, and were imaged through PET-CT at 12 h post-transplantation. Images are shown in FIGS. 3A and 3B. Mice were imaged at 24 h post-transplantation as shown in FIGS. 3C and 3D. Distribution of cells was comparable between single cell and spheroid animals. FIG. 3E

FIGS. 4A to 4D show changes in portal pressure upon administration of single cells or spheroids. Thrombic events are also illustrated. FIG. 4A shows that animals receiving single cells, 4.8-15.0 g, experienced mean portal pressure changes of 0.8-6.0 mmHg. No thrombus was noted. FIG. 4B shows that animals receiving spheroid hepatocytes, 9.1-10.8 g, experienced mean portal pressure changes of 10.9-12.5 mmHg. FIGS. 4C and 4D show portal vein thrombi in two animals, which were treated with enoxaparin for seven days.

FIGS. 5A and 5B show immunohistochemical staining of liver biopsies that were performed at six months post-transplantation on a pig. Multiple FAH-positive nodules (dark) were identified by immunohistochemistry on one spheroid-treated pig (FIG. 5A) and one single cell-treated pig (FIG. 5B).

FIG. 6 shows the presence of lentiviral (LV)-hFAH by genomic PCR in multiple samples of thirteen different tissue types at 48 hours (pig 1, top) and 60 days (pig2, bottom) post-treatment with in vivo injection of lentivirus. Four different liver samples were analyzed: liver left lateral, liver right medial, liver left medial, and liver right lateral and are shown in the first 4 lanes. LV-hFAH was detected in each of the liver samples. No detectable lentiviral integration was found in any of the extra-hepatic tissues analyzed.

FIGS. 7A and 7B show liver biopsy showing multiple FAH+ nodules at day 60 post-treatment of FAH−/− pigs subjected to direct percutaneous portal vein infusions of a lentiviral vector carrying the human Fah gene under control of the hepatocyte-specific alpha1-antitrypsin promoter at a dose of 2×10¹⁰ transducing units at six weeks of age.

FIGS. 8A to 8F show the levels of AST (FIG. 8A), alkaline phosphatase (FIG. 8B), tyrosine (FIG. 8C), ammonia (FIG. 8D), total bilirubin (FIG. 8E), and international normalized ratio (INR) (FIG. 8F) were more similar to the levels in wild type controls than the FAH−/− pigs subjected to direct percutaneous portal vein infusions of a lentiviral vector carrying the human Fah gene under control of the hepatocyte-specific alpha1-antitrypsin promoter at a dose of 2×10¹⁰ transducing units at six weeks of age at 5 months after transduction.

FIGS. 9A and 9B show weight graphs for pig 1 (FIG. 9A) and pig 2 (FIG. 9B) FAH−/− pigs subjected to direct percutaneous portal vein infusions of a lentiviral vector carrying the human Fah gene under control of the hepatocyte-specific alpha1-antitrypsin promoter at a dose of 2×10¹⁰ transduction units at six weeks of age showing complete NTBC independence at days 98 and 78 post-treatment, respectively.

FIG. 10 shows autologous hepatocyte transplantation by direct injection of a suspension of single hepatocytes transduced with the sodium iodine symporter (NIS) for incorporation of radiotracer and carrying the human Fah gene under control of the hepatocyte-specific alpha1-antitrypsin promoter into lymph nodes at a dose of 2×10¹⁰ transduction units at five weeks of age. All mesenteric lymph nodes were injected. The figure shows PET-CT images of ⁸⁹Zr-labeled hepatocytes at 6, 54, and 150 h post-transplantation into pig mesenteric lymph nodes. Representative axial images showing presence of ⁸⁹Zr-labeled hepatocytes in the mesenteric lymph nodes within 6 h of transplantation in a single pig, and engraftment maintained at nearly a week post-transplantation. At the 54 and 150 h time-points, hepatocyte engraftment is observed within the liver as well as in the mesenteric lymph nodes.

FIG. 11 shows long-term presence of ectopic liver tissue in mesenteric lymph nodes. PET-CT images of NIS-labeled hepatocytes at 104, 177, and 203 days post-transplantation into mesenteric lymph nodes. Representative coronal images showing persistent engraftment of NIS-labeled hepatocytes in the mesenteric lymph nodes in pigs 265 and 268. Mesenteric lymph nodes are outlined in white; stomach is outlined in dark line and kidney is outlined in dark line; bladder is labeled as “B”.

FIG. 12 shows histological confirmation of ectopic hepatocyte presence in mesenteric lymph nodes at 140 and 235-239 days post-transplantation. Pig 265: IHC showing presence of FAH-positive hepatocytes at days 140 (top left, middle) and 239 (top right) post-transplantation. Immunofluorescence at day 239 showing cytokeratin-18 (CK18) positive hepatocytes and reticular fibroblasts (ER-TR7) on Hoechst background (bottom left, middle), and cytokeratin-7 (CK7) positive bile duct cells (bottom right). Pig 268: IHC showing presence of FAH-positive hepatocytes at days 140 (top left, middle) and 235 (top right) post-transplantation. Immunofluorescence at day 235 showing cytokeratin-18 (CK18) positive hepatocytes and reticular fibroblasts (ER-TR7) on Hoechst background (bottom left, middle), and glutamine synthetase (GS) positive hepatocytes around CD31-positive endothelial cells forming possible central vein (bottom right).

FIG. 13 shows weight gain of treated pigs. Weight stabilization of pigs 272 and 278 demonstrating NTBC-independent growth at days 97 and 131 post-transplantation, respectively.

FIG. 14 shows the nucleic acid sequence coding for human fumarylacetoacetate hydrolase as inserted into a lentiviral vector. The nucleic acid coding for fumarylacetoacetate hydrolase is located at nucleotides 5545 to 6806, starting with the highlighted ATG and ending with the highlighted TGA. The hepatic control center enhancer and alpha 1 anti trypsin promoter are also shown. The alpha 1 anti trypsin promoter is located at nucleotides 5535 to 5545.

FIG. 15 shows the primer pairs used to show the integration of the lentiviral vector including the human fumarylacetoacetate hydrolase gene into tissues or cells.

FIG. 16 shows the nucleic acid sequence and polypeptide sequence for human fumarylacetoacetate hydrolase utilized in the vectors described herein.

FIGS. 17A and 17B show the schematic representation of a lentiviral vector and experimental process involved in ex vivo gene therapy. (FIG. 17A) The porcine Fah cDNA is under control of the human TBG promoter. LTR, long terminal repeat; Ψ, psi packaging sequence; RRE, Rev-responsive element; cPPT, central polypurine tract; WPRE, Woodchuck hepatitis virus posttranscriptional regulatory element; LTR/AU3, 3′ long terminal repeat with deletion in U3 region. (FIG. 17B) Four steps are involved in this process: 1) partial hepatectomy, 2) primary hepatocyte isolation through collagenase perfusion, 3) ex vivo gene therapy with a lentiviral vector carrying the porcine Fah gene, and 4) retransplantation of autologous hepatocytes in single cell or spheroid form.

FIGS. 18A to 18C show morphology and viability of spheroids, and differences in transduction efficiency between single cell and spheroid hepatocytes. (FIG. 18A) Light microscopy image of spheroids. (FIG. 18B) Inverted fluorescence microscopy image of spheroid hepatocytes using the Fluoroquench viability stain prior to transplantation in pig 897; light spots represents live cells and darker spots represents necrotic cells. (FIG. 18C) Ex vivo transduction of primary pig single cell hepatocytes and hepatocyte spheroids with LV-GFP at MOIs of 500 and 2000 LPs per cell and quantification of GFP-positive cells detected by flow cytometry 96 h after transduction. Data are means±SD (n=3 replicates for single cells, 2 replicates for spheroids). *P<0.01.

FIGS. 19A to 19D show microPET-CT images of ⁸⁹Zr-labeled single cell and spheroid hepatocytes at 2, 24, and 48 h post-transplantation in mice. (FIG. 19A) Percent of initial administered dose present in liver, spleen, and intestine at the 2, 24, and 48 h time points in spheroid and single cell mice. (FIG. 19B) Coronal images of a representative single cell mouse showing near homogenous distribution. (FIGS. 19C and 19D) Coronal images of two representative spheroid mice, showing intragroup differences in cell biodistribution.

FIGS. 20A to 20C show PET-CT images of ⁸⁹Zr-labeled single cell and spheroid hepatocytes at 6, 30, and 66 h post-transplantation in pig. (FIG. 20A) Quantification of liver, spleen, stomach, and intestine standardized uptake values (SUV). (FIGS. 20B and 20C) Axial views of five sequential PET-CT cuts in 19.6 mm slices at the 6, 30, and 66, h after single cell hepatocyte (B) or spheroid hepatocyte (C) transplantation in the pig. Stomach and spleen contours are outlined.

FIGS. 21A and 21B show Weight gain and corresponding histology. (FIG. 20A) Weight stabilization of pigs 896 (single cell) and 888 (spheroid) demonstrating similar growth and NTBC independence patterns. (FIG. 20B) Representative liver tissues stained for FAH from pigs 896 (top) and 888 (bottom) at 9-10 months post-transplantation, showing near-complete liver repopulation with FAH-positive hepatocytes in pig 888. H&E-stained and Masson's trichrome-stained serial liver sections are also provided.

FIG. 22 shows biochemical correction of treated pigs. Normalization of tyrosine, aspartate aminotransferase (AST), alkaline phosphatase (ALP), ammonia, and total bilirubin levels at the time of euthanization in all animals. Mean±SD are shown for experimental animal cohort alongside historical wild-type and untreated Fah−/− controls. p-values are provided for experimental animal cohort compared to untreated Fah−/− controls.

FIG. 23 shows liver repopulation with FAH-positive hepatocytes. Pig 278: IHC showing presence of FAH-positive hepatocytes in mesenteric lymph node (top image 1), liver (top image 2), and expanded view of FAH-positive hepatocytes in liver (top image 3 at day 239 post-transplantation; Trichrome (top image 4) and expanded view (top image 5) showing hepatocellular damage and fibrosis in FAH-negative areas of the liver at day 239 post-transplantation. Pig 270: IHC showing minimal presence of FAH-positive hepatocytes in mesenteric lymph node (bottom image 1) and complete liver repopulation with FAH-positive cells (bottom image 2; expanded view bottom image 3) at day 241 post-transplantation; Trichrome (bottom image 4; expanded image bottom image 5) showing resolution of fibrosis with minimal residual scarring in FAH-repopulated livers at day 241 post-transplantation.

FIGS. 24A to 24D show lentiviral integration profiles in lymph node vs. liver hepatocytes. (FIG. 24A) Relative integration frequency in exons, introns, intergenic regions, 3-prime UTRs, 5-prime UTRs, inside transcription units, and outside transcriptions units for transplanted hepatocytes that remained in lymph nodes and transplanted hepatocytes that migrated to the liver. (FIG. 24B) Relative integration frequency in CpG rich promoter regions vs. other areas of the genome for transplanted hepatocytes that remained in lymph nodes and transplanted hepatocytes that migrated to the liver. (FIG. 24C) Relative integration frequency based on distance upstream and downstream of transcription start sites for lymph node (left) and liver (right) hepatocytes. (FIG. 24D) Relative integration frequency in tumorigenic genes vs. other areas of the genome for lymph node (left) and liver (right) hepatocytes.

FIG. 25 shows weight stabilization of pigs 265, 268, and 270. NTBC-independent weight gain was achieved at days 158, 133, and 163, respectively, after three to six cycles on the drug.

FIG. 26 shows lentiviral integration profile in lymph node vs. liver hepatocytes by chromosome. Relative gene density per chromosome (top) compared to relative integration density per chromosome by tissue type (bottom).

FIG. 27 shows density of integration sites across each chromosome partitioned into 1 Mb windows. The density is calculated by counting the number of integration points overlapping each 1 Mb window, divided by the size of the window. In each chromosome, dark areas indicate highly condensed regions, light areas indicate least condensed regions. Red areas indicate centromeres.

FIG. 28 show the correlation of integration site density and gene density in each chromosome.

FIGS. 29A to 29F show characterization of integration sites in transcription units and regulatory elements. (FIG. 29A) percent of integration sites across features, not normalized by the genomic prevalence of each feature. The transcription units contain 77% of the integration sites. Within the transcription units, the introns show the highest percentage of integration sites. (FIG. 29B) Relative integration frequency across features normalized by the genomic prevalence of each feature. Relative integration frequency above 1 indicates integration events were enriched in that feature. (FIG. 29C) Relative integration frequency across features excluding integration sites in FAH and SerpinAl. (FIG. 29D) average percentage of integration points across features in transcripts divided into quadrants of transcript from 5′ to 3′. (FIG. 29E) percentages of integration sites around transcription start sites (TSS). (FIG. 29F) comparison of relative frequency of integration sites across known CpG islands and non-CpG regions of the genome.

FIGS. 30A and 30B show relative integration frequency of lentivirus in actively transcribed genes categorized into 5 groups based on publicly available expression RPKM values in (A) HepG2 Hepatocellular Carcinoma from the American Type Culture Collection (ATCC). B) Normal Liver Tissues from the Genotype-Tissue Expression (GTEx) project. The categories are labelled in increasing order of expression levels, with 1 representing group-1, lowest expression and 5 representing group-5, highest expression.

FIGS. 31A to 31C shows comparison of integration frequency in tumorigenic genes and the entire genome. (FIG. 31A) the distribution of integration points in features in the whole genome (blue) and the Mayo Large NGS Tumor Gene Panel (red) comprising 745 Tumor Associated Genes. (FIG. 31B) integrations per million base pairs (IPM) in the coding regions of tumor-associated genes (from Mayo large NGS Tumor Panel) and coding regions of other non-tumor-associated genes. (FIG. 31C) Percentages of integration sites around transcription start sites of tumor panel genes.

FIG. 32 shows PCR validation of 1× and 5×NGS reads. PCR forward primer was designed in junction of virus-host DNA. Reverse primer was from downstream host DNA. 10 sequences from 1× read and 5× reads were randomly selected. All real sequences NGS reported should show a clear unique band.

FIGS. 33A to 33D show LV-FAH transduction into wild type and chemical liver injury mice. A. schematic representation of the lenti vector expressing FAH. B. Diagram of study design with body weights by group showing the timing of the initiator DEN dose on Day 1 (darker dotted line—1), administration of the LV-FAH on Day 8/9 (gray dotted line—2) and CCl4 repeat dosing phase of the study from Days 43-106 (shaded gray region). Although body weights were similar throughout the study, the LV+DC group (Group 4) was statistically lower than Control (Group 1) from Day 71 through the end of the study (*, p<0.05). C. Bar graph showing the body weights on Day 1 (left bar of each set) compared to the end of the study (right bar of each set) for each group. Notably, DC and LV groups were statistically heavier than control and LV+DC at the initiation of the study despite age matching (* p<0.001 compared to control and LV+DC groups). D. Analysis of genomic DNA demonstrated that the LV group and DC group demonstrated 1-2% of cells were integrated with LV-FAH by the end of the study, while control and DC groups had negligible lentiviral vector genomes detected, similar to negative control sample (* p<0.01 compared to control and DC groups).

FIGS. 34A to 34D show serum chemistry parameters of liver and kidney effects of chemical liver injury and LV-FAH. Results of serum chemistry analysis at the end of the study (Day 106) from mice by groups for A. aspartate aminotransferase, B. Alanine aminotransferase, C. blood urea nitrogen, and D. creatinine. Results indicate increased liver enzymes in the DC-treated groups, regardless of LV and slight elevations in kidney parameters in some animals in LV-treated groups. Where applicable, upper and lower assay limits are indicated for AST and Creatinine by dashed lines, respectively.

FIGS. 35A to 35D show organ to body weight ratios for chemical liver injury in LV-FAH treated mice. Organ weights were collected at necropsy and normalized to terminal body weights for A. heart, B. spleen, C. kidney, and D. liver. Heart to body weight ratio was slightly decreased in liver injury mice regardless of LV-FAH administration. Spleen and liver weight ratios were greatest in the combination (LV+DC) group. p<0.05 compared to * control, ** control and LV, *** control, DC, and LV.

FIGS. 36A and 36B show markers of tumorigenicity and regeneration from chemical liver injury in LV-FAH treated mice. A. alpha fetoprotein was increased in chemical liver injury, regardless of administration of LV (note logarithmic Y axis). B. Percent of ki-67 positive cells in random sections of liver from mice for each group. C. Representative panels of ki-67 immunohistochemistry from mice from each group. Ki-67 staining was markedly increased in response to chemical liver injury, regardless of LV co-administration. p<0.05 compared to * control or ** control and LV.

FIGS. 37A to 37F shows biochemical analyses confirmed amelioration of metabolic disease. (FIG. 37A) Long-term weight data on pig Y842 from birth (day 0) though 532 days of age. Time on NTBC is represented by shaded bars. (B-E) Plasma or serum from the time of sacrifice was compared to historical wild type (n=4) and Fah^{-/-} untreated controls (n=5). (F) succinyl acetone (SUAC) in urine from the time of sacrifice was compared to historical wild type (n=3) and Fah^{-/-} untreated controls (n=4).

FIG. 38 Histological analyses revealed no variation in liver pathology associated with LV integration. (A-B) Representative low (left) and high (right) magnification images from pig Y842 at time of euthanasia after FAH IHC. (C-D) Representative low (left) and high (right) magnification images from pig Y842 at time of euthanasia after H&E staining. (E) Masson's Trichrome (MT) staining in a representative liver section at time of euthanasia. (F) Quantification of collagen staining was compared in the same animal from time of transplant (Pre), 1 year post transplantation biopsy (12 mo), and at time of euthanasia (31 mo). (G) Ki67-staining staining in a representative liver section at time of euthanasia in pig Y842. (H) Quantification of Ki67-positive hepatocyte nuclei staining was compared in pig Y842 and a 42-month old control pig.

### DETAILED DESCRIPTION

### Definitions

As used herein, “collecting” expanded human hepatocytes refers to the process of removing the expanded hepatocytes from an animal that has been injected or transplanted with isolated human hepatocytes (also referred to as a recipient animal). Collecting optionally includes separating the hepatocytes from other cell types. In one embodiment, the expanded human hepatocytes are collected from the liver of a Fah-deficient animal. In some examples, the expanded human hepatocytes are collected from the lymph nodes of a Fah-deficient pig.

As used herein, “coding for” refers to a nucleic acid sequence comprising triplets of four possible nucleotides that form codons that are translated into one of twenty possible amino acids of a polypeptide. A sequence of codons results in a corresponding sequence of amino acids that form all or part of a polypeptide.

As used herein, “Common-.gamma chain of the interleukin receptor (Il12rg)” refers to a gene encoding the common gamma chain of interleukin receptors. Il12rg is a component of the receptors for a number of interleukins, including IL-2, IL-4, IL-7 and IL-15 (Di Santo et al. Proc. Natl. Acad. Sci. U.S.A. 92:377-381, 1995). Animals deficient in Il12rg exhibit a reduction in B cells and T cells and lack natural killer cells. Also known as interleukin-2 receptor gamma chain.

As used herein, “cyclosporine” refers to an immunosuppressant compound that is a non-ribosomal cyclic peptide of 11 amino acids produced by the soil fungus Beauveria nivea. Cyclosporin A is used for the prophylaxis of graft rejection in organ and tissue transplantation. Cyclosporin A is also known as cyclosporine and ciclosporin.

As used herein, “Decreased liver function” refers to an abnormal change in any one of a number of parameters that measure the health or function of the liver. Decreased liver function is also referred to herein as “liver dysfunction.” Liver function can be evaluated by any one of a number of means well known in the art, such as, but not limited to, examination of liver histology and measurement of liver enzymes or other proteins, especially in the blood. For example, liver dysfunction can be indicated by necrosis, inflammation, fibrosis, oxidative damage or dysplasia of the liver. In some instances, liver dysfunction is indicated by hepatic cancer, such as hepatocellular carcinoma. Examples of liver enzymes and proteins that can be tested in the blood to evaluate liver dysfunction include, but are not limited to, alanine aminotransferase (ALT), aspartate aminotransferase (AST), bilirubin, alkaline phosphatase and albumin. Liver dysfunction also can result in generalized liver failure. Procedures for testing liver function are well known in the art, such as those taught by Grompe et al. (Genes Dev. 7:2298-2307, 1993) and Manning et al. (Proc. Natl. Acad. Sci. U.S.A. 96:11928-11933, 1999).

As used herein, “Fah-deficient” or “deficient in Fah” or “FAH−/−” refers to an animal, such as a pig, comprising a disruption in the Fah gene (such as an insertion, deletion or one or more point mutations), which results in a substantial decrease in, or the absence of, Fah mRNA expression and/or functional FAH protein or FAH activity.

As used herein, the term “loss of expression” of functional FAH protein does not refer to only a complete loss of expression, but also includes a substantial decrease in expression of functional FAH protein or FAH activity, such as a decrease of about 80%, about 90%, about 95% or about 99%. In some embodiments, the Fah-deficient animal comprises homozygous insertions in the Fah gene (such as an insertion that includes an in-frame stop codon). In some embodiments, the insertion is in exon 5 of Fah. In some embodiments, the Fah-deficient animal comprises homozygous deletions in the Fah gene. As one example, the homozygous deletion is in exon 5 of Fah. In another embodiment, the Fah-deficient animal comprises one or more point mutations in the Fah gene. Examples of suitable Fah point mutations are known in the art (see, for example, Aponte et al. Proc. Natl. Acad. Sci. U.S.A. 98(2):641-645, 2001).

As used herein, a “disruption” in a gene refers to any insertion, deletion or point mutation, or any combination thereof. In some embodiments, the disruption leads to a partial or complete loss of expression of mRNA and/or functional protein.

As used herein “Embryonic stem (ES) cells” refers to pluripotent cells isolated from the inner cell mass of the developing blastocyst. ES cells are pluripotent cells, meaning that they can generate all of the cells present in the body (bone, muscle, brain cells, etc.). Methods for producing murine ES cells can be found in U.S. Pat. No. 5,670,372. Methods for producing human ES cells can be found in U.S. Pat. No. 6,090,622, PCT Publication No. WO 00/70021 and PCT Publication No. WO 00/27995. Also contemplated herein are induced pluripotent stem cells (iPS cells), which are a type of pluripotent stem cell artificially derived from a non-pluripotent cell (such as an adult somatic cell) by inducing expression of certain genes, such as OCT3/4, SOX2, NANOG, LIN28, Klf4, and/or c-Myc (Yu et al., Science 318(5858):1917-1920, 2007; Takahashi et al., Cell 131(5):861-872, 2007). Thus far, iPS cells from mouse (Okita et al., Nature 448(7151):313-317, 2007), human (Yu et al., Science 318(5858):1917-1920, 2007; Takahashi et al., Cell 131(5):861-872, 2007), rat (Li et al., Cell Stem Cell 4(1):16-19, 2009), monkey (Liu et al., Cell Stem Cell 3(6):587-590, 2008) and pig (Esteban et al., J. Biol. Chem. Epub Apr. 21, 2009) have been reported.

As used herein, “Exogenous” refers to a non-endogenous isolated nucleic acid sequence or polypeptide obtained from a source outside of the subject or produced outside the subject. In some cases, an exogenous isolated nucleic acid is present as an extrachromosomal element in a portion of its cells or is stably integrated into its DNA (i.e., in the genomic sequence of most or all of its cells) or genome of a cell type, such as a hepatocyte. Exogenous isolated nucleic acid can be introduced into the germ line of transgenic animals by genetic manipulation of, for example, embryos or embryonic stem cells of the host animal according to methods well known in the art or introduced into a fetal, infant, or adult hepatocytes using the methods as described herein.

As used herein, “Expand” refers to increase in quantity. As used herein, “expanding” human hepatocytes refers to the process of allowing cell division to occur such that the number of human hepatocytes increases.

As used herein, “Fetus” refers to the unborn offspring of an animal in the postembryonic period.

As used herein, “FK506” refers to FK506, also known as tacrolimus or fujimycin, is an immunosuppressant drug. FK506 a 23-membered macrolide lactone first discovered in the fermentation broth of a Japanese soil sample that contained the bacteria Streptomyces tsukubaensis. This compound is often used after allogeneic organ transplant to reduce the activity of the patient's immune system and lower the risk of organ rejection. FK506 reduces T-cell and interleukin-2 activity. It is also used in a topical preparation in the treatment of severe atopic dermatitis (eczema), severe refractory uveitis after bone marrow transplants, and the skin condition vitiligo.

As used herein, “Fludarabine” refers to a purine analog that inhibits DNA synthesis. Fludarabine is often used as a chemotherapeutic drug for the treatment of various hematologic malignancies.

As used herein, “Fumarylacetoacetate hydrolase (FAH)” refers to a metabolic enzyme that catalyzes the last step of tyrosine catabolism. Mice having a homozygous deletion of the Fah gene exhibit altered liver mRNA expression and severe liver dysfunction (Grompe et al. Genes Dev. 7:2298-2307, 1993). Point mutations in the Fah gene have also been shown to cause hepatic failure and postnatal lethality (Aponte et al. Proc. Natl. Acad. Sci. U.S.A. 98(2):641-645, 2001). Humans deficient for Fah develop the liver disease hereditary tyrosinemia type 1 (HT1) and develop liver failure. Fah deficiency leads to accumulation of fumarylacetoacetate, a potent oxidizing agent and this ultimately leads to cell death of hepatocytes deficient for Fah. Thus, Fah-deficient animals can be repopulated with hepatocytes from other species, including humans.

As used herein, “functional polypeptide” refers to a polymer of amino acids that has the function of known polypeptide or protein. For example, a functional phenylalanine hydroxylase catalyzes the conversion of phenylalanine to tyrosine. An example of a polypeptide sequence for human phenylalanine hydroxylase is found at GenBank Accession No. NP_000268 and a nucleic acid coding for this polypeptide is found at GenBank Accession No. NM_000277. For example, a functional fumarylacetoacetate hydrolase catalyzes the conversion of 4-fumarylacetoacetate to acetoacetate. An example of a human polypeptide sequence for fumarylacetoacetate hydrolase is found at GenBank Accession No. NP_000128 and a nucleic acid coding for this polypeptide is found at GenBank Accession No. NM_000137 (nucleotides 165-1424). Other sequences for polypeptides and isolated exogenous nucleic acids for all or part of polypeptides can be found through GenBank or other sequence databases. In cases, where there is a mutation that leads to a polypeptide with reduced or lack of function, the polypeptide can be replaced with a functional polypeptide, including, for example, a wild type polypeptide.

As used herein, “Gestation” refers to the period of development from conception (fertilization of an oocyte) to birth. The gestation period for a pig is (on average) 112-115 days.

As used herein, “Hepatocyte” refers to a type of cell that makes up 70-80% of the cytoplasmic mass of the liver. Hepatocytes are involved in protein synthesis, protein storage and transformation of carbohydrates, synthesis of cholesterol, bile salts and phospholipids, and detoxification, modification and excretion of exogenous and endogenous substances. The hepatocyte also initiates the formation and secretion of bile. Hepatocytes manufacture serum albumin, fibrinogen and the prothrombin group of clotting factors and are the main site for the synthesis of lipoproteins, ceruloplasmin, transferrin, complement and glycoproteins. In addition, hepatocytes have the ability to metabolize, detoxify, and inactivate exogenous compounds such as drugs and insecticides, and endogenous compounds such as steroids.

As used herein, “Hereditary tyrosinemia type 1 (HT1)” refers to an error of metabolism, usually inborn, in which the body cannot effectively break down the amino acid tyrosine. HT1 is the most severe form of this disorder and is caused by a shortage of the enzyme fumarylacetoacetate hydrolase (FAH) encoded by the gene Fah found on human chromosome number 15. FAH is the last in a series of five enzymes needed to break down tyrosine. Symptoms of HT1 usually appear in the first few months of life and include failure to gain weight and grow at the expected rate (failure to thrive), diarrhea, vomiting, yellowing of the skin and whites of the eyes (jaundice), cabbage-like odor, and increased tendency to bleed (particularly nosebleeds). HT1 can lead to liver and kidney failure, problems affecting the nervous system, and an increased risk of liver cancer.

As used herein, “Immunodeficient” refers to lacking in at least one essential function of the immune system. As used herein, an “immunodeficient” animal is one lacking specific components of the immune system or lacking function of specific components of the immune system (such as, for example, B cells, T cells or NK cells). In some cases, an immunodeficient animal lacks macrophages. In some embodiments, an immunodeficient animal (such as a Fah-deficient pig) comprises one or more genetic alterations that prevent or inhibit the development of functional immune cells (such as B cells, T cells or NK cells). In some examples, the genetic alteration is in the Rag1, Rag2 or Il12rg gene. Thus, in some cases, the immunodeficient animal is Rag1.sup.−/−, Rag2.sup.−/− or Il2. sup.−/−.

As used herein, “Immunosuppressant” refers to any compound that decreases the function or activity of one or more aspects of the immune system, such as a component of the humoral or cellular immune system or the complement system. Immunosuppressants are also referred to as “immunosuppressive agents.” In particular embodiments of the disclosure, the immunosuppressant is FK506, cyclosporin A, fludarabine, mycophenolate, prednisone, rapamycin or azathioprine, or combinations thereof. Known immunosuppressants include, but are not limited to: (1) antimetabolites, such as purine synthesis inhibitors (e.g., azathioprine and mycophenolic acid), pyrimidine synthesis inhibitors (e.g., leflunomide and teriflunomide) and antifolates (e.g., methotrexate); (2) macrolides, such as FK506, cyclosporine A and pimecrolimus; (3) TNF-.alpha. inhibitors, such as thalidomide and lenalidomide; (4) IL-1 receptor antagonists, such as anakinra; (5) mammalian target of rapamycin (mTOR) inhibitors, such as rapamycin (sirolimus), deforolimus, everolimus, temsirolimus, zotarolimus and biolimus A9; (6) corticosteroids, such as prednisone; and (7) antibodies to any one of a number of cellular or serum targets.

As used herein, “Immunosuppression” refers to the act of reducing the activity or function of the immune system. Immunosuppression can be achieved by administration of an immunosuppressant compound or can be the effect of a disease or disorder (for example, immunosuppression that results from HIV infection or due to a genetic defect). In some cases, immunosuppression occurs as the result of a genetic mutation that prevents or inhibits the development of functional immune cells.

As used herein, “Isolated” refers to an “isolated” biological component, such as an isolated exogenous nucleic acid, protein (including antibodies) or organelle, has been substantially separated or purified away from other biological components in the environment (such as a cell) in which the component naturally occurs, i.e., other chromosomal and extra-chromosomal DNA and RNA, proteins and organelles. Isolated exogenous nucleic acids and proteins that have been “isolated” include isolated exogenous nucleic acids and proteins purified by standard purification methods. The term also embraces isolated exogenous nucleic acids and proteins prepared by recombinant expression in a host cell as well as chemically synthesized isolated exogenous nucleic acids.

As used herein, “Isolated exogenous nucleic acid” refers to a polymer of nucleotides including ribonucleic acid (RNA) and deoxyribonucleic acid (DNA). Nucleic acids can contain units of nucleotides called genes. A gene contains nucleotides that code for a polypeptide.

As used herein, “NTBC (2-nitro-4-trifluoro-methyl-benzoyl)-1, 3 cyclohexanedione)” refers to an inhibitor of 4-hydroxy-phenylpyruvate dioxygenase (HPPD). HPPD catalyzes the conversion of 4-hydroxyphenylpyruvate to homogentisate, the second step in tyrosine catabolism. Treatment with NTBC blocks the tyrosine catabolism pathway at this step and prevents the accumulation of succinylacetone, a pathognomonic metabolite that accumulates in Fah-deficient humans and animals.

As used herein, “Recombinase activating gene 1 (Rag1)” refers to a gene involved in activation of immunoglobulin V (D) J recombination. The RAG1 protein is involved in recognition of the DNA substrate, but stable binding and cleavage activity also requires RAG2. As used herein. “Recombinase activating gene 2 (Rag2)” refers to a gene involved in recombination of immunoglobulin and T cell receptor loci. Animals deficient in the Rag2 gene are unable to undergo V (D) J recombination, resulting in a complete loss of functional T cells and B cells (Shinkai et al. Cell 68:855-867, 1992).

As used herein, “Stem cell” refers to a cell having the unique capacity to produce unaltered daughter cells (self-renewal; cell division produces at least one daughter cell that is identical to the parent cell) and to give rise to specialized cell types (potency). Stem cells include, but are not limited to, embryonic stem (ES) cells, embryonic germ (EG) cells, germline stem (GS) cells, fetal stem cells. Hepatic stem cells, human mesenchymal stem cells (hMSCs), adipose tissue-derived stem cells (ADSCs), multipotent adult progenitor cells (MAPCs), multipotent adult germline stem cells (maGSCs) and unrestricted somatic stem cell (USSCs). The role of stem cells in vivo is to replace cells that are destroyed during the normal life of an animal. Generally, stem cells can divide without limit. After division, the stem cell may remain as a stem cell, become a precursor cell, or proceed to terminal differentiation. A precursor cell is a cell that can generate a fully differentiated functional cell of at least one given cell type. Generally, precursor cells can divide. After division, a precursor cell can remain a precursor cell, or may proceed to terminal differentiation. In one embodiment, the stem cells give rise to hepatocytes.

As used herein, “Subject” refers to living multi-cellular vertebrate organisms, a category that includes both human and non-human mammals, including pigs, as well as fetuses and live born mammals.

As used herein, “Vector” refers to an isolated nucleic acid molecule allowing insertion of exogenous isolated exogenous nucleic acid without disrupting the ability of the vector to integrate in a host cell. A vector can also include one or more selectable marker genes and other genetic elements. An integrating vector is capable of integrating itself into a host isolated exogenous nucleic acid. An expression vector is a vector that contains the necessary regulatory sequences to allow transcription and translation of inserted gene or genes.

As used herein, “wild type” refers to the dominant or common phenotype of a particular gene or characteristic in a species. In embodiments, the wild type gene or protein has an associated function.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for isolated exogenous nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Methods

A first aspect of the disclosure provides for methods of gene therapy to correct defective genes in liver cells. The methods described herein provide improved targeting of the expression of corrected gene and/or wild type gene to liver cells avoiding expression and/or integration into other cell types that could lead to detrimental side effects. A method of delivering an isolated exogenous nucleic acid coding for all or part of a polypeptide to the liver as disclosed herein comprises administering the isolated exogenous nucleic acid to hepatocytes of a subject, such as a fetus, infant, or older pediatric patient, by delivering the nucleic acid intrahepatically or via the portal vein, wherein the isolated exogenous nucleic acid codes for all or part of a polypeptide operably linked to a hepatocyte specific promoter, wherein the isolated exogenous nucleic acid integrates at the location of a nucleic acid coding for all or part of the polypeptide in the genome of the fetal hepatocyte. In embodiments, the isolated exogenous nucleic acid can be delivered in a hepatocyte or in a stem cell of the same species of the subject, especially those hepatocytes or stem cells obtained from or derived from the same fetus, infant, or older pediatric patient. In embodiments, the isolated exogenous nucleic acid or hepatocytes containing the isolated nucleic acid can be delivered to lymph nodes.

In other embodiments, the isolated exogenous nucleic acid comprises a vector that delivers the isolated exogenous nucleic acid to a hepatocyte. In certain embodiments, the vector integrates into the genome of a hepatocyte. In embodiments, once delivered to the hepatocyte, the isolated exogenous nucleic acid is expressed in the hepatocyte using a hepatocyte specific promoter. In embodiments, the promoter is an inducible promoter. In embodiments, use of a hepatocyte specific promoter provides for targeting the expression of the nucleic acid in hepatocytes rather than other cell types such as the brain, lungs, or heart.

### Isolated Exogenous Nucleic Acids

In embodiments, the methods of the disclosure employ an isolated exogenous nucleic acid that codes for all or a portion of a polypeptide. In embodiments, the polypeptide is one that has a decreased function in cells such as hepatocytes due to a genetic defect. In embodiments, the isolated exogenous nucleic acid comprises a vector. In specific embodiments, the vector is one that can integrate into the genome of the hepatocyte. In embodiments, the vector has a preference for integration based on sequence homology and optionally, a minimal preference for integration at tumorigenesis related genes. Non limiting examples of vectors include lentivirus, adeno associated virus, and retroviruses. In embodiments, the isolated exogenous nucleic acid that codes for all or part of a polypeptide can comprise double stranded oligonucleotides, single-strand oligonucleotides, triplex-forming oligonucleotides, RNA-DNA hybrids, or a small fragment DNA. In other embodiments, single-strand oligonucleotides, triplex-forming oligonucleotides, RNA-DNA hybrids, or a small fragment DNA codes for a sequence to correct a mutation in an intron that results in truncation or deletion of one or more exons of the polypeptide.

The targeting of isolated exogenous nucleic acid to hepatocytes can be improved by providing the nucleic acid intrahepatically, or via the portal vein. In embodiments, such delivery methods are targeted using ultrasound guided percutaneous delivery. In further embodiments, the portal pressure is monitored during delivery and stopped or paused if the portal pressure is 2 mm Hg, 3 mm Hg, 4 mm Hg, 5 mm Hg, 6 mm Hg, 7 mm Hg, or 8 mm Hg or greater than normal portal pressures. Normal portal pressures in infants or children are about 5 to 10 mm Hg. Once intraportal pressure returns to about 10 mm Hg or less, administration is resumed. In addition, the subject can be treated with an anticoagulant before, during, and/or after administration.

In embodiments, when a vector is administered, a dosage of about 10⁸ to 10¹¹ Transducing Units per/kg is employed. In embodiments, using a dose of less than about 10⁹ transfection units per gram of tissue minimizes the introduction of the vector into other cell types and/or tissues. In embodiments, it is desirable to minimize introduction of the vector into cell types and/or tissue that could cause unwanted side effects such as the heart, lungs, or brain. In addition, a vector can be selected in order to reduce immunogenicity. Subjects can be screened for existing immune responses to any of the viral vectors.

In embodiments, the targeting of the isolated exogenous nucleic acid to hepatocytes can be improved by providing the isolated exogenous nucleic acid in a stem cell or hepatocyte. In this case, vectors are not being administered systemically such that they come into contact with other cell types or tissues. In embodiments, a hepatocyte or stem cell comprising the isolated exogenous nucleic acid as described herein is administered at a dose of 10⁷ cells to about 10¹⁰ cells per subject. In certain embodiments, the transformed or transduced cells are administered as spheres. In other embodiments, the spheres have a diameter of about 50-100 mm. In embodiments, about 4 to 15 grams of single cell suspension is delivered to the animals. In other embodiments, about 9 to 12 grams of spheres are delivered to the animals with or without a pharmaceutical agent to prevent thrombosis, such as enoxaparin. In addition, employing an autologous stem cell or hepatocyte may decrease any immunogenicity associated with using vectors such as AAV.

In some cases, the isolated exogenous nucleic acid codes for a functional polypeptide that corresponds to a polypeptide that is lacking, defective, or has reduced function in a (e.g. less than 80% of the functional activity of the wild type polypeptide) in the subject. In such cases, the gene therapy involves gene addition to provide for a functional polypeptide in cells that do not have a functional polypeptide. In embodiments involving gene addition, the isolated exogenous nucleic acid comprises double stranded nucleic acid coding for a functional polypeptide. The function of the polypeptide can readily be tested using enzymatic assays or cell based assays. For example, polypeptides that are associated with metabolic pathways can be tested for catalytic activity. In another case, for cell signaling molecules, the functional polypeptide can be expressed in a cell and signaling activity measured in the cell. Function of polypeptides and assays for testing function of polypeptides are known to those of skill in the art. In embodiments a functional polypeptide has at least 85%, 86%, 87%, 88%, 89%, 90%, 91%, 92%, 93%, 94%, 95%, 96%, 97%, 98%, or 99% of the functional activity of a reference polypeptide or the wild type polypeptide.

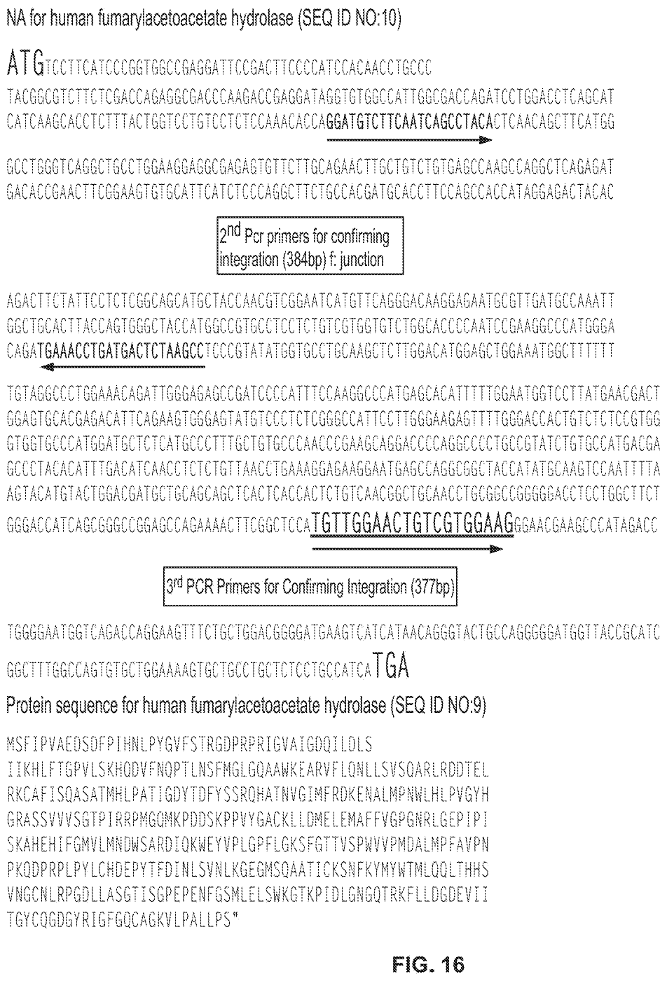

In specific embodiments, the isolated exogenous nucleic acid codes for a functional fumarylacetoacetate hydrolase, a functional tyrosine transaminase, a functional p-hydroxyphenyl pyruvate dioxegenase, a functional phenylalanine hydroxylase, or combinations thereof. Sequences for nucleic acid and polypeptides for these enzymes are known to those of skill in the art and can readily be identified using publicly available sequence databases. In some embodiments, the nucleic acid codes for a wild type polypeptide. In other embodiments, the polypeptide may have changes from the wild type sequence but still retain the function of the wild type polypeptide. Such changes can include amino acid substitutions, deletions, or insertions. In some cases, naturally occurring variants exist that can be utilized. In addition, all or a part of any leader sequence or preprotein sequence may not be present. In embodiments, a functional polypeptide has an amino acid sequence identity of at least 90%, 91%, 92%, 93%, 94%, 95%, 96%, 97%, 98%, or 99% sequence identity to a human reference polypeptide (as identified in GenBank or other databases) and retains the function of the polypeptide. For example, the nucleic acid sequence (SEQ ID NO: 8) and polypeptide sequence (SEQ ID NO: 9) for human fumarylacetoacetate hydrolase are shown in FIG. 16.

In other embodiments, the functional polypeptide is selected based on a lack of or reduced immunogenicity in the subject using methods known to those of skill in the art as described with respect to the use of therapeutic proteins. The generation of antibodies to polypeptides causing undesirable immunogenicity is dependent on the presence of T helper cell epitopes, which have been identified by in vitro methods using blood cells from human donors. In silico methods that identify T cell epitopes have been developed. These methods are relatively inexpensive and allow the mapping of epitopes from virtually all human leukocyte antigen molecules derived from a wide genetic background. In other embodiments, for example, 84 different mutations in fumarylacetoacetate hydrolase have been identified and any one or more of these mutations may be corrected using the isolated exogenous nucleic acid described herein. In embodiments, a subject is screened for the type and location of the mutation in the polypeptide and an isolated exogenous nucleic acid coding for all or part of a polypeptide can be designed for that mutation. The polypeptide encoded by the isolated exogenous nucleic acid can be screened for immunogenicity using the in vitro and/or in silico methods for identifying T cell epitopes as described herein.

In other embodiments, the isolated exogenous nucleic acid comprises single-strand oligonucleotides, triplex-forming oligonucleotides, RNA-DNA hybrids, or a small fragment DNA. The single-strand oligonucleotides, triplex-forming oligonucleotides, RNA-DNA hybrids, or a small fragment DNA codes for a portion of a polypeptide that needs to be corrected. In such cases, the constructs comprise at least the corresponding nucleotides that code for the genetic defect but have the corrected sequence that provides a functional polypeptide. Such a genetic defect can be found in introns, exons, or both. In other embodiments, 84 different mutations in fumarylacetoacetate hydrolase have been identified and any one or more of these mutations may be corrected using the vectors described herein. In embodiments, a subject is screened for the type and location of the mutation in the polypeptide so a gene repair construct can be designed for that mutation.

In embodiments, the isolated exogenous nucleic acid comprises a promoter operably linked to a nucleic acid coding for all or a part of a polypeptide. In embodiments, the promoter is a spleen focus forming virus promoter, a cytomegalovirus promoter, or an alpha1-antitrypsin (AAT) promoter. In embodiments, a promoter is one that is expressed predominantly in hepatocytes such as the alpha1-antitrypsin (AAT) promoter and thyroxin binding globulin (TBG) promoter. In embodiments, the promoter is the human alpha 1 antitrypsin promoter.

In embodiments, a promoter can be an inducible promoter. In embodiments, a promoter is selected that has low genotoxicity. Standard in vitro genotoxicity assays include the bacterial reverse mutation assay, the in vitro mammalian chromosomal aberration test, the in vitro mammalian cell gene mutation test [Hprt] and [MLA/tk]), and the in vitro mammalian cell micronucleus test. Confirmatory in vivo tests are also known. (Corvi et al., Food and Chemical Toxicity, 2017, 106:600.) In embodiments, a low genotoxic promoter is the alpha1 antitrypsin promoter. In embodiments, the promoter reduces integration near tumorigenic genes. For example, the alpha anti trypsin promoter has a lower risk of being associated with hepatocellular carcinoma than the thyroxin binding globulin promoter (Chandler et al., J. Clin. Investigation 125:870(2015).

In embodiments, the promoter is modified from its native form by reducing the size of the promoter region. In embodiments, the promoter has 1000 bp or less, 900 bp or less, 800 bp or less, 700 bp or less, 600 bp or less, 500 bp or less, 400 bp or less, 300 bp or less, or 200 bp or less. In embodiments, the promoter is a chimeric promoter with enhancer sequences different than that normally associated with the promoter.

In embodiments, a hepatocyte specific promoter is selected that provides expression of all or part of the polypeptide of at least 99%, 95, 90%, 80%, 70%, 60%, 50%, 40%, 30% of the amount of expression when expression of the polypeptide is under the control of the CMV promoter.

In embodiments, the isolated nucleic acid construct comprises an enhancer sequence to provide for enhancement of expression. In embodiments, enhancer sequences are known to those of skill in the art. In embodiments, the enhancer sequence provides for enhanced expression in hepatocytes, for example, the enhancer sequences of the alpha1-antitrypsin (AAT) promoter or the hepatocyte control region.

In embodiments, the isolated exogenous nucleic acid is prepared all or in part synthetically or all or in part by combining nucleic acid elements recombinantly using methods known to those of skill in the art.

### Lentiviral Vector

Another aspect of the disclosure provides a lentiviral vector that provides for specific expression in hepatocytes. In embodiment, the lentiviral vector comprises an isolated exogenous nucleic acid coding for all or part of a polypeptide operably linked to a promoter. In embodiments, the promoter is a hepatocyte specific promoter. In embodiments, the vector further comprises hepatocyte specific enhancer sequences.

In embodiments, the vector has deletions from the wild type to render it replication deficient and includes elements to reduce virulence. In certain embodiments, the lentiviral vector has a backbone comprising the 5′ long terminal repeat (LTR) followed by the psi packaging sequence (P) followed by the rev responsive element (RRE), the central polypurine tract (cPPT), the woodchuck hepatitis virus posttranscriptional regulatory element (WPRE), and the 3′ LTR with all or part of the U3 region deleted. A nucleic acid comprising a hepatic control region enhancer (HCR) operably linked to the human alpha 1 anti trypsin promoter (AAT) operably linked to the human Fah gene (SEQ ID NO: 8) was inserted into the Lentiviral backbone between the cPPT site and the WPRE site using ClaI and BamHI restriction sites for the AAT promoter, followed by newly introduced BlpI and EcoRI for the FAH gene.

In some embodiments, lentiviral vectors are replication incompetent. Lentiviral vectors comprise RNA genome sequences coding gag, env, and pol proteins. In embodiments, one or more of RNA genome sequences coding for rev, tat, vpr, vif, vpx, vpu, or nef are absent or disrupted. In other embodiments, one or more of the gag, pol, or env are absent or disrupted. In embodiments, modified or wild type long terminal repeats (LTR) are present at the 5′ and 3′ ends of the viral vector. The LTRs comprise three elements: U3, R and U5. U3 is derived from the sequence unique to the 3′ end of the RNA. R is derived from a sequence repeated at both ends of the RNA and U5 is derived from the sequence unique to the 5′end of the RNA. For the viral genome, the site of transcription initiation is at the boundary between U3 and R in the left hand side LTR and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

In embodiments, a lentivirus vector comprises a LTR region that has reduced promoter activity relative to wild-type LTR, in that such constructs provide a self-inactivating (SIN) biosafety feature. Self-inactivating vectors are ones in which the production of full-length vector RNA in transduced cells in greatly reduced or abolished altogether. This feature greatly minimizes the risk that replication-competent recombinants will emerge. Furthermore, it reduces the risk that that cellular coding sequences located adjacent to the vector integration site will be aberrantly expressed. Furthermore, an SIN design reduces the possibility of interference between the LTR and the promoter that is driving the expression of the transgene.

Self-inactivation is preferably achieved through in the introduction of a deletion in the U3 region of the 3′ LTR of the vector DNA, i.e., the DNA used to produce the vector RNA. Thus, during reverse transcription, this deletion is transferred to the 5′ LTR of the proviral DNA. It is desirable to eliminate enough of the U3 sequence to greatly diminish or abolish altogether the transcriptional activity of the LTR, thereby greatly diminishing or abolishing the production of full-length vector RNA in transduced cells. However, it is generally desirable to retain those elements of the LTR that are involved in polyadenylation of the viral RNA, a function spread out over U3, R and U5. Accordingly, it is desirable to eliminate as many of the transcriptionally important motifs from the LTR as possible while sparing the polyadenylation determinants. In embodiments, a modification of a LTR comprises a deletion of the LTR TATA box (e.g., deletions from −418 to −18), without significant reductions in vector titers. These deletions render the LTR region substantially transcriptionally inactive in that the transcriptional ability of the LTR in reduced to about 90% or lower. In preferred embodiments the LTR transcription is reduced to about 95% to 99%. Thus, the LTR may be rendered about 90%, 91%, 92%, 93%, 94%, 95%, 96%, 97%, 98%, to about 99% transcriptionally inactive.

For certain applications, for example, in the case of promoters that are only modestly active in cells targeted for transduction it is desirable to employ a posttranscriptional regulatory sequence positioned to promote the expression of the transgene. One type of posttranscriptional regulatory sequence is an intron positioned within the expression cassette, which may serve to stimulate gene expression. However, introns placed in such a manner may expose the lentiviral RNA transcript to the normal cellular splicing and processing mechanisms. Thus, in particular embodiments it may be desirable to locate intron-containing transgenes in an orientation opposite to that of the vector genomic transcript.

In embodiments, a method of enhancing transgene expression is through the use of a posttranscriptional regulatory element which does not rely on splicing events, such as the posttranscriptional processing element of herpes simplex virus, the posttranscriptional regulatory element of the hepatitis B virus (HPRE) or that of the woodchuck hepatitis virus (WPRE), which contains an additional cis-acting element not found in the HPRE. The regulatory element is positioned within the vector so as to be included in the RNA transcript of the transgene, but outside of stop codon of the transgene translational unit. It has been found that the use of such regulatory elements is particularly preferred in the context of modest promoters, but may be contraindicated in the case of very highly efficient promoters.

In some applications, the vector is replication deficient (or replication defective) to avoid uncontrolled proliferation of the virus in the subject to be treated. In such instances mammalian cell lines are selected which have been engineered, either by modification of the producer cell's genome to encode essential viral functions or by the co-infection of the producer cell with a helper virus, to express proteins complementing the effect of the sequences deleted from the viral genome. In the production of minimal vector systems, the producer cell is engineered (either by modification of the viral genome or by the use of helper virus or cosmid) to complement the functions of the parent virus enabling replication and packaging into virions in the producer cell line. Stable packaging lines are known to those of skill in the art and are described, for example, at Sanber et. al., Scientific reports 5, article 9021, (2015).

In other embodiments, the lentiviral vector is psuedotyped by expanding the vector in a packaging cell line with another isolated nucleic acid coding for another viral glycoprotein, such as the VSV glycoprotein. In other embodiments, other viral glycoproteins can be utilized such as from Sindbis virus, Ross River virus, or Baculovirus GP64. In embodiments, utilizing other glycoproteins increases the targeting of the vector to hepatocytes or lymph nodes.

In embodiments, a lentivirus vector may also contain a nucleic acid sequence coding for a reporter molecule and/or coding for a selectable marker. Such reporter molecules provide for identification of cells that have incorporated the lentivirus vector. Reporter molecules include green fluorescent protein, and/or luciferase. Many types of lentivirus vectors with reporters and selectable markers are commercially available.

In embodiments, the lentivirus vector comprises an isolated exogenous nucleic acid coding for all or part of a polypeptide as described herein. In embodiments, the isolated exogenous nucleic acid codes for all or part of a functional polypeptide that corrects a genetic defect in a hepatocyte. In embodiments, the isolated exogenous nucleic acid can be delivered in a hepatocyte or in a stem cell of the same species of the subject, especially those hepatocytes or stem cells obtained from or derived from the fetus or the infant. In other embodiments, the isolated exogenous nucleic acid comprises a lentiviral vector that delivers the isolated exogenous nucleic acid to a hepatocyte or lymph node.

In certain embodiments, the vector integrates into the genome of a hepatocyte or lymph node cell. In embodiments, a vector is selected with a preference for integration based on sequence homology, and optionally, with a minimal preference for integration at tumorigenesis related genes and/or CpG islands. In embodiments, the vector integrates more often into the nucleic acid region coding for the polypeptide that has a gene mutation in the genome of the hepatocyte, via an apparently homology-guided mechanism. In embodiments, once delivered to the hepatocyte or lymph node, the isolated exogenous nucleic acid is expressed in the hepatocyte using a hepatocyte specific promoter and/or lymph node specific promoter. In embodiments, the promoter is an inducible promoter. In embodiments, use of a hepatocyte specific promoter provides for targeting the expression of the nucleic acid in hepatocytes or lymph nodes rather than other cell types such as the brain or heart.

These methods and isolated exogenous nucleic acids are useful to correct gene defects in the liver such as inherited diseases of the liver. Inherited diseases of the liver include but are not limited to tyrosinemia, phenyl ketonuria, Wilson's disease, or alpha 1 anti trypsin deficiency.

In embodiments, a lentiviral vector is useful in a method for treating hereditary tyrosinemia. In embodiments the lentiviral vector comprises an isolated exogenous nucleic acid coding for all or part of a functional fumarylacetoacetate hydrolase, a functional tyrosine transaminase, a functional p hydroxyphenyl pyruvate dioxegenase, or combinations thereof, under control of a hepatocyte specific promoter. An example of a sequence of an isolated exogenous nucleic acid coding for all or part of a functional fumarylacetoacetate hydrolase is provided in SEQ ID NO: 8.

In embodiments, the hepatocyte specific promoter is an alpha1 anti trypsin promoter. In embodiments, the nucleic acid also comprises enhancer sequences. In embodiments, the promoter is a chimeric promoter with the enhancer sequences different from the enhancer sequences normally associated with the promoter.

In embodiments, any of the isolated exogenous nucleic acids as described herein can be utilized in the method of treating.

In embodiments, a lentiviral vector is useful in a method for treating phenyl ketonuria. In embodiments, the lentiviral vector comprises an isolated exogenous nucleic acid that codes for all or part of a functional phenylalanine hydroxylase under control of a hepatocyte specific promoter. In embodiments, the hepatocyte specific promoter is an alpha1 anti trypsin promoter. In embodiments, the nucleic acid also comprises enhancer sequences. In embodiments, the promoter is a chimeric promoter with the enhancer sequences different from the enhancer sequences normally associated with the promoter. In embodiments, any of the isolated exogenous nucleic acids as described herein can be utilized in the method of treating.

In some cases, genetic defects can be due to many different mutations in a gene. In such cases, a biological sample is isolated and analyzed for the specific mutation in the gene of interest. Once the mutation is identified, an isolated exogenous nucleic acid can be designed to replace or repair the specific mutation. For example, fumarylacetoacetate hydrolase in hereditary tyrosinemia can have one of 84 different mutations. Identification of a mutation resulting in decreased function of fumarylacetoacetate hydrolase can be corrected using an isolated exogenous nucleic acid coding for a wild type fumarylacetoacetate hydrolase or using a specifically designed functional polypeptide. In embodiments, any specifically designed polypeptide has at least 90%, 91%, 92%, 93%, 94%, 95%, 96%, 97%, 98%, or 99% sequence identity to a wild type or reference polypeptide. In embodiments, any specifically designed polypeptide has at least 85%, 86%, 97%, 88%, 89%, 90%, 91%, 92%, 93%, 94%, 95%, 96%, 97%, 98%, or 99% of the functional activity of a reference polypeptide or the wild type polypeptide. In other embodiments, any specifically designed polypeptide is screened for immunogenicity.

### Administration

The routes of administration of the isolated exogenous nucleic acid are selected to provide delivery to the liver or lymph nodes. In embodiments, the route of delivery is intrahepatic (using vasculature or nonvasculature) or via the portal vein. In embodiments, delivery by the portal vein is ultrasound guided percutaneous delivery. In further embodiments, the portal pressure is monitored during delivery and stopped or paused if the portal pressure is 2 mm Hg, 3 mm Hg, 4 mm Hg, 5 mm Hg, 6 mm Hg, 7 mm Hg, or 8 mm Hg or greater than normal portal pressures. Normal portal pressures in infants or children are about 5 to 10 mm Hg. In embodiments, the subject can also be treated with an anticoagulant before, during, and/or after administration. Once intraportal pressure returns to about 10 mmHg or less, administration is resumed.

In other embodiments, the route of delivery is injection into mesenteric lymph nodes. In embodiments, a composition comprises an isolated exogenous nucleic acid or cells containing the isolated exogenous nucleic acid in a variety of pharmaceutically acceptable excipients. If cells are utilized, a composition comprises a dosage and a pharmaceutical excipient to keep the portal vein pressure at 14 mm Hg or less. In embodiments, the dosage of cells or spheres is about 10⁸ to 10¹⁰ cells, in a single injection. Multiple injections may be made as necessary based on the indication that the metabolic defect has not been fully corrected by examining biomarkers.

In addition, vectors or isolated exogenous nucleic acid can be packaged in liposomes or in combination with molecules that enhance uptake into liver cells, such as polyethylene imine, and chitosan.

### Subjects

In embodiments, the subject is a human. In embodiments, the subject is a fetus, a neonate, an infant, or an older pediatric patient. In embodiments, the fetus is at least 4 weeks old, 5 weeks old, 6 weeks old, 7 weeks old, 8 weeks old, 9 weeks old, 10 weeks old, 11 weeks old, 12 weeks old, 13 weeks old, 14 weeks old, 15 weeks old, 16 weeks old, 17 weeks old, 18 weeks old, 19 weeks old, 20 weeks old, 21 weeks old, 22 weeks old, 23 weeks old, 24 weeks old, 25 weeks old, 26 weeks old, 27 weeks old, 28 weeks old, 29 weeks old, 30 weeks old, 31 weeks old, 32 weeks old, 33 weeks old, 34 weeks old, 35 weeks old, 36 weeks old, 37 weeks old, 38 weeks old, 39 weeks old, or 40 weeks old. In other embodiments, the fetus is at least 25 weeks old. In embodiments the human is a neonate that is at least 1 day old, 2 days old, 3 days old, 4 days old, 5 days old, 6 days old, 7 days old, 8 days old, 9 days old, 10 days old, 11 days old, 12 days old, 13 days old, 14 days old, 15 days old, 16 days old, 17 days old, 18 days old, 19 days old, 20 days old, 21 days old, 22 days old, 23 days old, 24 days old, 25 days old, 26 days old, 27 days old, or 28 days old. In certain embodiments, the human is an infant at least 29 days old. In other embodiments, the infant is 1 year old or less. In embodiments, the human is an older pediatric patient at least 1 year old or greater, and/or 18 years old or less.

In embodiments, the fetus, a neonate, an infant, or an older pediatric patient has been screened for a genetic defect. In some embodiments, the genetic defect is a defect in a metabolic pathway and the specific mutation or mutations resulting in the decreased function of the polypeptide can be identified. The identification of the gene and the particular mutation allows for the design of a corrected gene to be included in the isolated exogenous nucleic acid or utilization of the wild type gene.

### Cells

In another aspect of the disclosure, the isolated exogenous nucleic acid is contained with a hepatocyte from the same species as the subject. In embodiments, the hepatocyte is obtained from or derived from the fetus or the infant. The hepatocytes are transduced or transformed with the isolated exogenous nucleic acid that provides for expression of a functional polypeptide and expanded. In embodiments, the expansion of the hepatocytes can be conducted in a genetically altered animal that is immunosuppressed such as a pig homozygous for a disruption in the fumarylacetoacetate hydrolase (Fah) gene such that the disruption results in loss of expression of functional FAH protein, wherein the pig exhibits decreased liver function. These pigs are referred to as fumarylacetoacetate hydrolase (FAH)-deficient pigs or FAH −/−. Production of such pigs and methods of expanding hepatocytes in them are described in U.S. Pat. Nos. 9,000,257 and 9,485,971, which are hereby incorporated by reference In embodiments, any type of corrected gene or wild type gene in the isolated exogenous nucleic acid sequence in the hepatocytes can be expanded in the FAH−/− pig in combination with a nucleic acid coding for a functional fumarylacetoacetate hydrolase as these cells have a competitive advantage as compared to the hepatocytes of the FAH−/− pig.

In addition, the FAH−/− pig can be immunosuppressed either by knocking out genes or by treating the animal with an immunosuppressive agent. Any suitable immunosuppressive agent or agents effective for achieving immunosuppression in a pig can be used. Examples of immunosuppressive agents include, but are not limited to, FK506, cyclosporin A, fludarabine, mycophenolate, prednisone, rapamycin and azathioprine. Combinations of immunosuppressive agents can also be administered. In some embodiments, the one or more immunosuppressive agents are administered to the Fah-deficient pig at least about 2 days prior to human hepatocyte transplantation. Immunosuppression may be continued for a period of time, for example for a portion of or the entire life of the pig. Although the use of immunosuppressive drugs is exemplified for obtaining immunodeficient animals, also contemplated are genetic alterations that result in a lack of a specific component of the immune system, or a lack of functionality of a specific component of the immune system (such as a lack of functional B, T and/or NK cells). In some embodiments, the genetic alteration is in the Rag1, Rag2 or Il2rg gene. Specific cells of the immune system (such as macrophages or NK cells) can also be depleted. Methods of depleting particular cell types are known in the art. Moreover, the two disclosed methods of expanding human hepatocytes in Fah-deficient pigs can be combined. Fetal transplantation can be carried out to induce tolerance to the human hepatocytes, thus permitting the administration of a larger dose of human hepatocytes postnatally.

In embodiments, a method comprises expanding human hepatocytes in vivo by transplanting human hepatocytes into an immunodeficient FAH−/− pig and allowing the human hepatocytes to expand. In embodiments, the FAH−/− pig also lacks a functional gene for Rag1, Rag2, Il2rg gene, or combinations thereof. In embodiments, the human hepatocyte is obtained from or derived from the fetus or the infant, and as such, are considered autologous to the fetus or infant. The hepatocytes are transduced or transformed with the isolated exogenous nucleic acid that provides for expression of a functional polypeptide to correct the genetic defect in the infant or fetus as well as a functional fumarylacetoacetate hydrolase.

In other embodiments, a method comprises expanding human hepatocytes in vivo by transplanting human hepatocytes into an immunodeficient phenylalanine hydroxylase (PAH)−/− pig and allowing the human hepatocytes to expand. In embodiments, the PAH−/− pig also lacks a functional gene for Rag1, Rag2, Il2rg gene, or combinations thereof. In embodiments, the human hepatocyte is obtained from or derived from the fetus or the infant, and as such, are considered autologous to the fetus or infant. The hepatocytes are transduced or transformed with the isolated exogenous nucleic acid that provides for expression of a functional polypeptide to correct the genetic defect in the infant or fetus as well as a functional phenylalanine hydroxylase. The length of time for hepatocyte expansion can vary and will depend on a variety of factors, including the number of hepatocytes originally transplanted, the number of human hepatocytes desired following expansion and/or the desired degree of liver repopulation with the human hepatocytes. In some cases, these factors will be dictated by the desired use of the hepatocytes or the desired use of the Fah-deficient pig or PAH deficient pig engrafted with the human hepatocytes. In some embodiments, the human hepatocytes are expanded in the Fah-deficient pig or PAH deficient pig for at least about 3 days, at least about 5 days, at least about 7 days, at least about 2 weeks, or at least about 4 weeks, at least about 2 months, at least about 3 months, at least about 4 months, at least about 5 months, at least about 6 months, at least about 7 months, at least about 8 months, at least about 9 months, at least about 10 months or at least about 11 months. In particular examples, the human hepatocytes are expanded in the Fah-deficient pig for at least 7 days. In other examples, the human hepatocytes are expanded in the Fah-deficient pig or FAH deficient pig for at least 6 months. In some examples, the human hepatocytes are expanded in the Fah-deficient pig or PAH deficient pig no more than 12 months.

In some embodiments, the FAH−/−-deficient pig or PAH deficient pig is administered an agent that inhibits, delays or prevents the development of liver disease in the pig. Administration of such an agent is necessary to prevent liver dysfunction and/or death of the animal prior to repopulation of the animal with healthy (FAH-expressing or PAH expressing) hepatocytes. The agent can be any compound or composition known in the art to inhibit liver disease. One such agent is 2-(2-nitro-4-trifluoro-methyl-benzoyl)-1, 3 cyclohexanedione (NTBC), but other pharmacologic inhibitors of phenylpyruvate dioxygenase, such as methyl-NTBC can be used. NTBC (or a similar compound) is administered to regulate the development of liver disease in the Fah-deficient pig. The dose, dosing schedule and method of administration can be adjusted as needed to prevent liver dysfunction in the Fah-deficient pig. In some embodiments, the FAH−/−-deficient pig is further administered NTBC for at least two days, at least three days, at least four days, at least five days or at least six days following hepatocyte transplantation. In some embodiments, the FAH−/−-deficient pig is further administered NTBC for at least about one week, at least about two weeks, at least about three weeks, at least about four weeks, at least about one month, at least about two months, at least about three months, at least about four months, at least about five months, or at least about six months.

In embodiments, once expanded, the hepatocytes are collected from the animal model, transduced, transformed, or transfected with an isolated exogenous nucleic acid as described herein, and delivered to the subject. In other embodiments, the hepatocytes are transduced, transformed, or transfected with an isolated exogenous nucleic acid as described herein, expanded in vivo, collected, and delivered to the subject. In embodiments, the hepatocytes are delivered intrahepatically or via the portal vein. In other embodiments, the hepatocytes are delivered to mesenteric lymph nodes. In embodiments, the transduced or transformed hepatocytes can be delivered as single cells or in spheres. In certain embodiments, spheres range in size from 50 to 100 mm.

In embodiments, the isolated nucleic acid introduced into a hepatocyte derived from the subject can have a further alteration in order to provide a selective advantage to those transduced cells. In embodiments, the FAH gene is added to the isolated nucleic acid in combination with the transgene that corrects another metabolic defect such as PKU. Other gene alterations in hepatocytes or the isolated nucleic acid providing selective advantage include overexpression of Forkhead boxM1 transcriptional activator or expression of Bcl-2.

In yet other embodiments, the isolated exogenous nucleic acid is contained within a stem cell. In embodiments, the stem cell is an adult stem cell, a hepatic stem cell, a fetal stem cell, or an embryonic stem cell of the same species as the subject. In embodiments, the stem cell is obtained from or derived from the same species of fetus or infant. In embodiments the stem cell is obtained from or derived from the same fetus or infant. In embodiments, the stem cells are from adipose tissue, bone marrow, and/or skin cells.

In embodiments, the stem cells are obtained from the fetus, infant, or older pediatric patient and then expanded in vitro. Stem cells can be expanded in vitro using methods known to those of skill in the art. (Snykers et al., In vitro differentiation of Embryonic and Adult Stem Cells into Hepatocytes: State of the Art, Stem Cells 2009, 27(3):577-605.) In embodiments, stem cells are cultured in a serum free medium including one or more growth factors. The combination of growth factors can provide a cell that can differentiate into hepatocytes. In embodiments, the stem cells are contacted with the isolated exogenous nucleic acid coding for all or part of a polypeptide as described herein. The stem cells are transduced, transfected or transformed with the exogenous nucleic acid as described herein so that the stem cells express a functional polypeptide.

A composition comprises an effective amount of transduced or transformed expanded stem cells and a pharmaceutically acceptable excipient. An effective amount of stem cells is about 10⁸ to 10¹² cells per kg.

### Method of Treatment

These methods and isolated exogenous nucleic acids are useful to correct gene defects in the liver such as inherited diseases of the liver. Inherited diseases of the liver include (but are not limited to) tyrosinemia, phenyl ketonuria, Wilson's disease, or alpha 1 anti trypsin deficiency.

In embodiments, a method for treating hereditary tyrosinemia comprises administering an isolated exogenous nucleic acid to hepatocytes of a subject, such as a fetus, infant, or older pediatric patient by delivering the isolated exogenous nucleic acid intrahepatically or via the portal vein, wherein the isolated exogenous nucleic acid codes for all or part of a functional fumarylacetoacetate hydrolase, a functional tyrosine transaminase, a functional p hydroxyphenyl pyruvate dioxegenase, or combinations thereof, under control of a hepatocyte specific promoter. In other embodiments, the isolated exogenous nucleic acid is delivered to mesenteric lymph nodes. In embodiments, the isolated exogenous nucleic acid is contained within a human hepatocyte derived from the subject to be treated.

In embodiments, the isolated exogenous nucleic acid comprises a vector. In embodiments, the hepatocyte specific promoter is a human alpha1 anti trypsin promoter. In embodiments, any of the isolated exogenous nucleic acids as described herein can be utilized in the method of treating. In embodiments, the human fumarylacetoacetate hydrolase has a nucleic acid sequence of SEQ ID NO: 8 and a polypeptide sequence of SEQ ID NO: 9.

In embodiments, a method for treating phenyl ketonuria comprises administering an isolated exogenous nucleic acid to hepatocytes of a subject, such as a fetus, infant, or older pediatric patient by delivering the isolated exogenous nucleic acid intrahepatically or via the portal vein, wherein the isolated exogenous nucleic acid codes for all or part of a functional phenylalanine hydroxylase under control of a hepatocyte specific promoter. In other embodiments, the isolated exogenous nucleic acid is delivered to mesenteric lymph nodes. In embodiments, the isolated exogenous nucleic acid is contained within a human hepatocyte derived from the subject to be treated.

In embodiments, the isolated exogenous nucleic acid comprises a vector. In embodiments, the hepatocyte specific promoter is an alpha1 anti trypsin promoter. In embodiments, any of the isolated exogenous nucleic acids as described herein can be utilized in the method of treating. In embodiments, the isolated nucleic acid comprises additional genes or genetic elements that provide for a selective advantage of transformed cells in the liver. For example, a FAH gene, Forkhead transcriptional activator and/or Bcl-2.

In some cases, genetic defects can be due to many different mutations in a gene. In such cases, a biological sample is isolated and analyzed for the specific mutation in the gene of interest. Once the mutation is identified, an isolated exogenous nucleic acid can be designed to replace or repair the specific mutation. For example, fumarylacetoacetate hydrolase in hereditary tyrosinemia can have one of 84 different mutations. Identification of a mutation resulting in decreased function of fumarylacetoacetate hydrolase can be corrected using an isolated exogenous nucleic acid coding for a wild type fumarylacetoacetate hydrolase or using a specifically designed functional polypeptide. In embodiments, any specifically designed polypeptide has at least 90%, 91%, 92%, 93%, 94%, 95%, 96%, 97%, 98%, or 99% sequence identity to a wild type or reference polypeptide. In embodiments, any specifically designed polypeptide has at least 85%, 86%, 97%, 88%, 89%, 90%, 91%, 92%, 93%, 94%, 95%, 96%, 97%, 98%, or 99% of the functional activity of the wild type polypeptide. In certain embodiments, the wild type gene is utilized. In other embodiments, any specifically designed polypeptide is screened for immunogenicity.

In embodiments, once the isolated exogenous nucleic acid is delivered to the hepatocytes, correction of the genetic defect can be monitored through biochemical analysis of liver function and tyrosine levels, histological analyses of liver fibrosis and clinically through the animal's ability to thrive off the protective drug NTBC/nitisinone. The levels of aspartate amino transferase (AST), alkaline phosphatase, tyrosine, ammonia, total bilirubin, and international normalized ratio (INR) is measured in in vivo treated animals, wild type control animals, and FAH−/− animals.

In embodiments, the methods as described herein for delivering an isolated exogenous nucleic acid coding for all or part of a polypeptide can comprise delivery of the isolated exogenous nucleic acid as described herein to lymph nodes, especially the mesenteric lymph nodes. In embodiments, a method for treating hereditary tyrosinemia comprises administering an isolated exogenous nucleic acid to lymph nodes of a subject, such as a fetus, infant, or older pediatric patient by delivering the isolated exogenous nucleic acid directly to the lymph nodes, wherein the isolated exogenous nucleic acid codes for all or part of a functional fumarylacetoacetate hydrolase, a functional tyrosine transaminase, a functional p hydroxyphenyl pyruvate dioxegenase, or combinations thereof, under control of a hepatocyte specific promoter.

In embodiments the human fumarylacetoacetate hydrolase has a nucleic acid sequence of SEQ ID NO:8 and a polypeptide sequence of SEQ ID NO: 9. In embodiments, the isolated exogenous nucleic acid is contained within a human hepatocyte derived from the subject to be treated.

In embodiments, a method for treating phenyl ketonuria comprises administering an isolated exogenous nucleic acid to lymph nodes of a subject, such as a fetus or infant, by delivering the isolated exogenous nucleic acid directly to lymph nodes, wherein the isolated exogenous nucleic acid codes for all or part of a functional phenylalanine hydroxylase under control of a hepatocyte specific promoter. In embodiments, the isolated exogenous nucleic acid is contained within a human hepatocyte derived from the subject to be treated.

The present disclosure will now be further described by way of the following non-limiting examples, provided for illustrative purposes only.

### Example 1

This example is directed to gene therapy of a murine model of hereditary tyrosinemia type 1 (HT1) through in utero gene therapy, as well as to evaluate lentiviral vector biodistribution and integration profile after in utero administration.

The Fah^{5981SB} mouse models hereditary tyrosinemia (HTI) by bearing a single N-ethyl-N-nitrosourea-induced point mutation in the final nucleotide of exon 8 within the Fah gene. (Aponte J L et al., Point mutations in the murine fumarylacetoacetate hydrolase gene: Animal models for the human genetic disorder hereditary tyrosinemia type 1. Proc Natl Acad Sci USA. 2001; 98:641-645.) This point mutation creates a premature downstream stop codon and exon 8 loss, ultimately leading to formation of truncated, unstable FAH protein that is degraded. Fah^{5981SB} mice die as neonates from acute liver failure if 2-(2-nitro-4-trifluoromethylbenzoyl)-1, 3-cyclohexanedione (NTBC), a highly potent inhibitor of the enzyme 4-hydroxyphenylpyruvate dioxygenase, is not continually administered in the drinking water. NTBC treatment at 4 mg/mL rescues the phenotype and prevents acute hepatocellular and renal injury. Discontinuation of NTBC provides an accurate model of HTI. Mice develop liver and renal disease within 10 days, which progresses to full end-stage liver disease and death within 6-8 weeks. (Grompe M et al., Pharmacological correction of neonatal lethal hepatic dysfunction in a murine model of hereditary tyrosinaemia type I. Nat Genet. 1995; 10:453-460).

### Methods

Direct fetal intrahepatic injections of a lentiviral vector carrying either the green fluorescent protein (GFP) under control of the spleen focus-forming virus (SFFV) promoter or the luciferase gene under control of the cytomegalovirus (CMV) promoter were performed in wild-type mice. Injections were performed at day 15 of gestation and 3×10⁸ TU/fetus. Mothers and fetuses that received GFP were euthanized 5-7 days after injection and their tissues sent for immunohistochemical (IHC) analysis. Mothers and fetuses that received luciferase were imaged with the Xenogen IVIS-200 system 3-5 days after injection.

In utero delivery of LV-GFP was then performed in a sow at gestational day 65 using 10⁸-10⁹ TU/fetus.

### Results and Conclusions

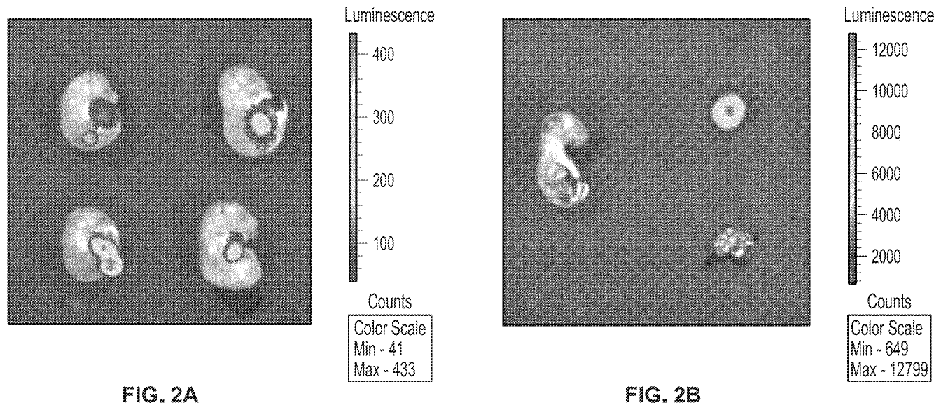

No evidence of GFP expression was found through IHC analysis of maternal tissues. Luciferase expression in mouse mothers was limited to the uterus, and in pups luciferase was preferentially expressed in the liver. (FIGS. 2A and 2B). PCR analysis performed on fetal/maternal pig tissues after LV-GFP delivery demonstrated no evidence of lentiviral vector integration in maternal tissues, and vector presence only in liver and kidney in piglets.

The results show that the lentiviral vector administered intrahepatically in utero was specifically targeted to the liver in the pups and did not affect tissues of the mother except the uterus.

### Example 2

This example is directed to gene therapy of a murine model of hereditary tyrosinemia type 1 (HT1) through in utero gene therapy.

### Methods

### Lentiviral Vector

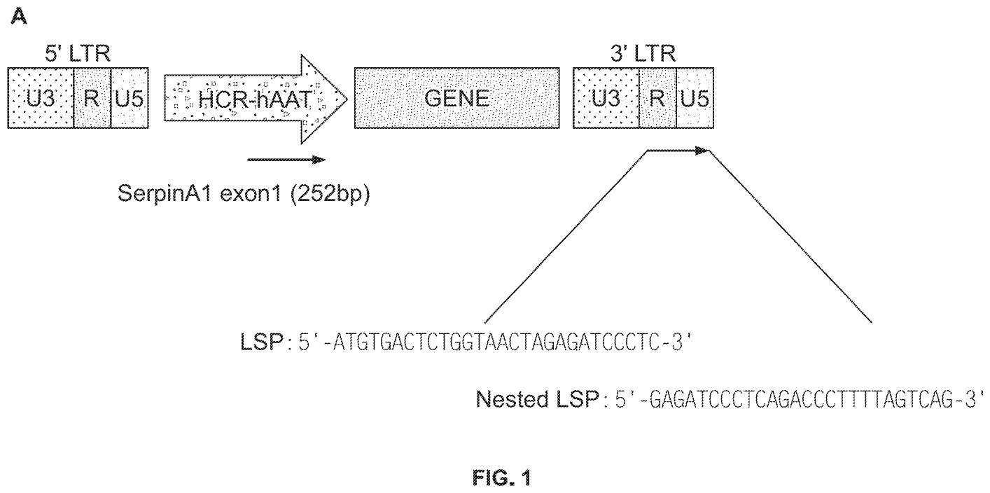

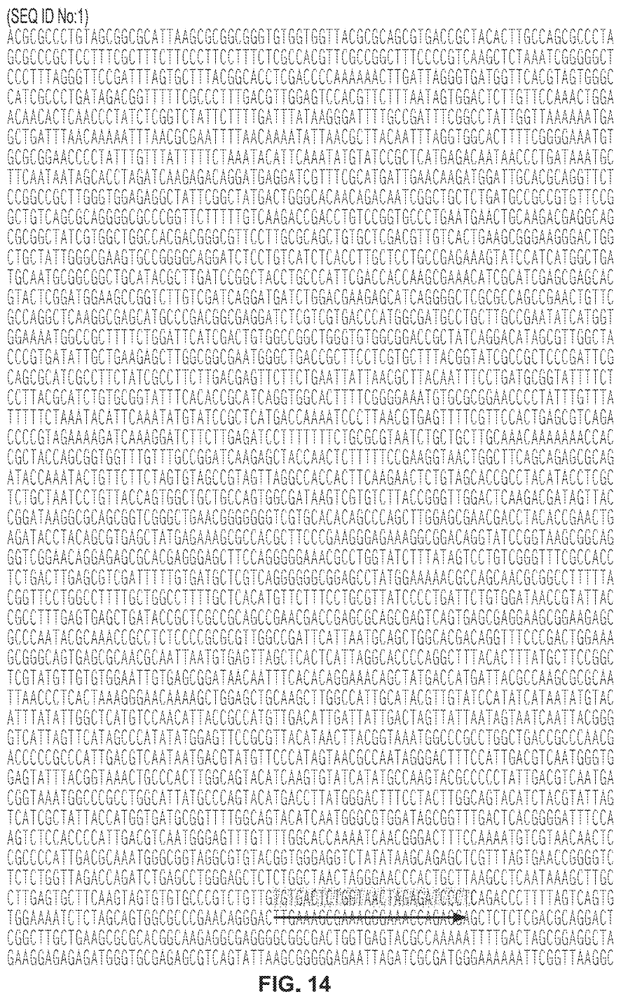

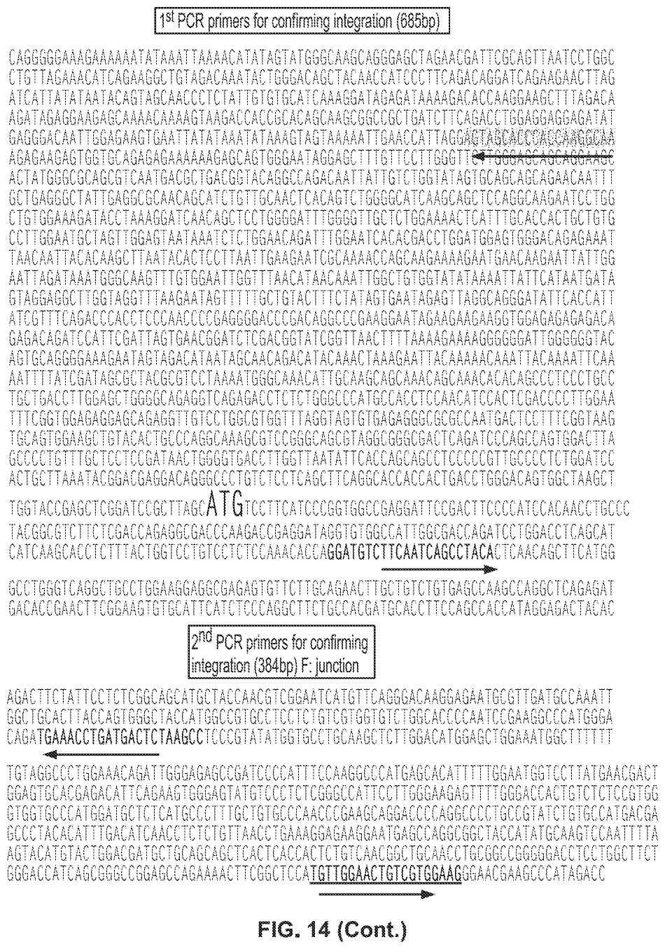

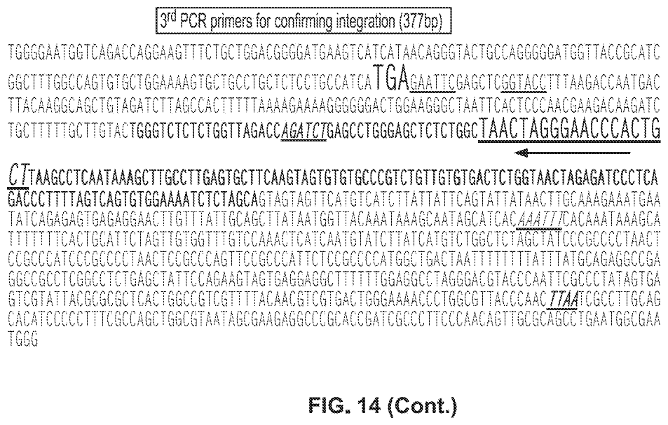

A lentiviral vector was constructed as follows. The lentiviral backbone contains the 5′ long terminal repeat (LTR) followed by the psi packaging sequence( ) followed by the rev responsive element (RRE), the central polypurine tract (cPPT), the woodchuck hepatitis virus posttranscriptional regulatory element (WPRE), and the 3″ LTR with all or part of the U3 region deleted. The lentiviral backbone can be obtained from commercial sources. FIG. 1 shows a nucleic acid comprising a hepatic control region enhancer (HCC) operably linked to the alpha 1 anti trypsin promoter (AAT) operably linked to the human Fah gene inserted into the Lentiviral backbone between the cPPT site and the WPRE site using ClaI and BamHI restriction sites for the AAT promoter, followed by newly introduced BlpI and EcoRI for the FAH gene. (SEQ ID NO: 1) See FIG. 14.

### Administration

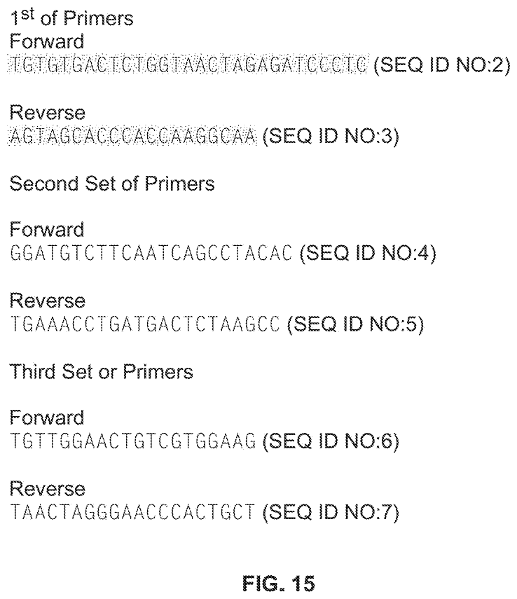

Intrahepatic injections were made in the mouse model of HT1 (commercially available from Jackson Laboratories) with a lentiviral vector carrying the human fumarylacetoacetate hydrolase (FAH) gene under control of the hepatocyte-specific alpha1-antitrypsin (AAT) promoter. Mothers were maintained on NTBC/nitinisone (Sigma Aldrich) treatment throughout pregnancy until the time of weaning, at which point administration was discontinued in the pups. Vector integration profile was studied through real-time PCR screening and next generation sequencing on eight different maternal and fetal tissue types. The sequence of the PCR primers are shown in FIG. 15.

### Results and Conclusions

Treated Fah−/− pups demonstrated the ability to maintain healthy NTBC-independent growth curves as soon as two weeks after weaning. Fourteen treated Fah^{-/-} pups demonstrated maintenance of NTBC-independent growth, with 10 (71%) not requiring any NTBC cycling. (data not shown) Complete liver repopulation by FAH-positive hepatocytes was seen on IHC at one month, together with tyrosine level normalization. Initial real-time PCR analysis demonstrated higher lentiviral vector integration in fetal liver, with lower vector copy numbers detected in other tissues. (data not shown) We show that in utero lentiviral gene therapy can correct FAH-deficient mice.

### Example 3

A pig model of hereditary tyrosinemia type 1 (HT1) was treated with ex vivo gene delivery via intraportal transplantation of spheroid suspension hepatocytes as compared to single cell suspensions. Using single cells provide advantages in that they are smaller is suspension, dedifferentiate in vitro, and can be used on the same day as harvest. In contrast, spheres have a higher cell density, higher efficiency of vector transduction, and are more stable in vitro.

### Materials and Methods

### Animals

For biodistribution experiments, Fox Chase SCID beige mice (Charles River Laboratories International, Inc., Wilmington, Mass.) and heterozygous Fah^{+/-} pigs were used. Fah^{-/-} pigs were produced in a 50% Large White and 50% Landrace pig as previously described. NTBC mixed in food was administered at a dose of 1 mg/kg/day with a maximum of 25 mg/day. All animals remained on NTBC until the time of transplantation, after which NTBC administration was discontinued to support expansion of the corrected cells. After hepatocyte transplantation, all animals were monitored daily for loss of appetite or any other clinical signs of morbidity. Animals were weighed daily for the first two weeks post-operatively and weekly thereafter. If loss of appetite, weight loss, or any other signs of morbidity occurred, NTBC treatment was reinitiated for seven days. Animals were cycled on and off NTBC in this fashion to stimulate expansion of corrected FAH-positive cells. For biodistribution experiments, female SCID beige mice and male heterozygous Fah^{+/-} pigs were used. Male and female Fah^{-/-} pigs were used in equal numbers for ex vivo gene therapy experiments.

### Liver Resection and Hepatocyte Isolation

Six-week-old (15-20 kg) pigs underwent a laparoscopic partial hepatectomy involving the left lateral lobe under inhaled general anesthesia with 1-3% isoflurane. Resection volumes represented 15 to 20% of the total liver mass. An upper midline incision was made for placement of a 12 mm port using an open Hasson technique, through which a 5 mm laparoscope (Stryker, Kalamazoo, Mich.) was passed. The abdomen was insufflated with CO2 to provide adequate visualization, and two additional 5 mm ports were placed. The liver vasculature was identified and isolated, at which point the parenchymal resection was performed using an Endo GIA stapler with a 45 mm vascular load (Covidien, Dublin, Ireland). When parenchymal resection was complete, the liver section was retrieved using an Endo Catch bag (Covidien, Dublin, Ireland), and adequate homeostasis was ensured prior to port removal and incision closure. This liver section was then perfused ex vivo through the portal vein with a two-step perfusion system to isolate hepatocytes as previously described.

### Biodistribution of Radiolabeled Hepatocytes

For biodistribution experiments, hepatocytes in single cell or spheroid form were radiolabeled in suspension with ⁸⁹Zr with synthon ⁸⁹Zr-DBN at 27° C. for 45 min in Hanks Buffered Salt Solution.

### Hepatocyte Transduction

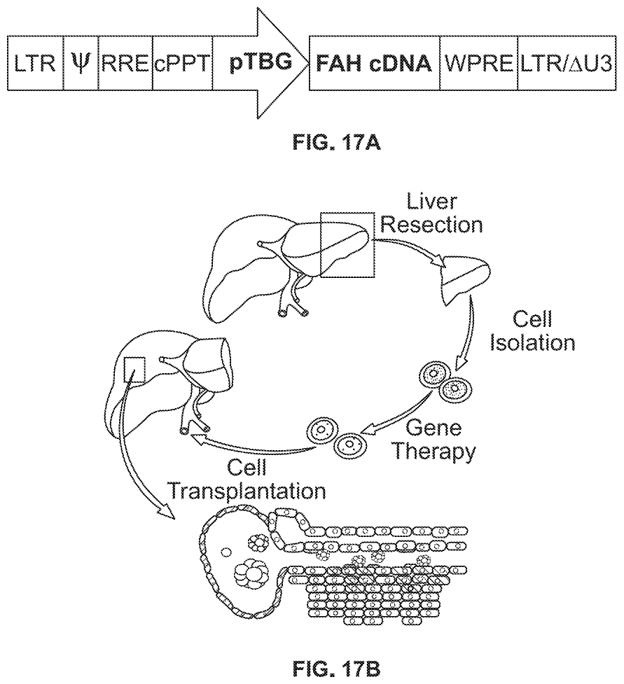

Hepatocytes were transduced in suspension at a multiplicity of infection (MOI) of 20 transduction units (TU) with a lentiviral vector carrying the porcine Fah cDNA under control of the human thyroxine-binding globulin (TBG) promoter and two copies of the human al-microglobulin/bikunin enhancer (FIGS. 17A and 17B). Hepatocytes from pigs randomized to receive single cell suspension were transduced in suspension for two hours before transplantation. Hepatocytes from pigs randomized to receive hepatocyte spheroids were transduced in suspension for 18-24 h, while spheroid formation was taking place by means of a rocking technique. Spheroid viability was assessed through inverted epifluorescent microscopy (Axiovert 135 TV, Carl Zeiss Inc., Thornwood, N.Y.) using the Fluoroquench fluorescent viability stain (One Lambda). Spheroid diameter was measured by use of a Multisizer3 with 560 μm aperture (Beckman Coulter, Fullerton, Calif.). Spheroid vs. single cell hepatocyte ex vivo transduction efficiency was assessed through transduction with a lentiviral vector expressing green fluorescent protein (GFP) at MOIs of 0, 500, and 2000 lentiviral particles (LPs) per cell. GFP-positive cells were then quantified by flow cytometry at 96 h post-transduction.

### Hepatocyte Transplantation in Mice

In mice, cell transplants were performed intrasplenically. Intrasplenic injections were performed through an open technique, where a small incision was made in the left upper quadrant of the abdomen, the spleen was visualized by partial evacuation from the abdomen, and cells were delivered directly into the splenic parenchyma via a 27- or 28-gauge 0.5-in needle.

### Hepatocyte Transplantation in Pigs

Wild-type and Fah^{-/-} pigs were randomized to receive autologous transplantation of either single cell hepatocytes or spheroid hepatocytes through ultrasound-guided percutaneous portal vein infusion. Single cell animals were kept under general anesthesia post-operatively until the time of transplantation, approximately 4 h later; spheroid animals were recovered post-operatively and re-anesthesized for transplantation the following day. A schematic representation of the experimental process is presented in FIG. 16. The portal vein was identified using a 2 to 5 MegaHz transducer (Fujifilm SonoSite, Inc., Bothell, Wash.), and an 18-gauge 5-in needle was directed towards the main portal vein prior to its bifurcation for manual infusion of hepatocytes. Portal pressures were monitored continuously during transplantation, and infusion was paused if pressures increased more than 8 mmHg above the upper limit of normal. When portal pressures did not return to baseline after pausing infusion for five minutes, the injection was discontinued and the actual doses administered are reported. Heparinization of the cell solution was performed at 70 U/kg of recipient weight. Ultrasound was used to evaluate for the presence of thrombotic events after transplantation.

### Positron Emission Tomography-Computed Tomography (PET-CT) Imaging and Analysis

In mice, imaging was performed on the Inveon MicroPET-CT (Siemens Medical Solutions USA, Inc., Malvern, Pa.) at 2, 24, and 48 h post-transplantation. CT was performed at 80 kEv, 500 uA, with 250 ms/projection, 180 projections, bin 4. The effective pixel size was 94.59 um. PET was performed at 10 min acquisition, OSEM2D reconstruction with Fourier rebinning, 4 iterations. In pigs, imaging was performed on the high-resolution GE Discovery 690 ADC PET/CT System (GE Healthcare, Chicago, Ill.) at 6, 30, and 66 h post-transplantation. CT was performed at 120 kV and 150 mA, with tube rotation of 0.5 s and pitch of 0.516. PET was performed as a two-bed acquisition with 10 min per bed and 17 slice overlap, resulting in a 27-cm axial field of view. All post reconstruction analysis was performed using PMOD software (PMOD Technologies LLC, Zirich, Switzerland). Activities were decay-corrected and normalized to standardized uptake value (SUV) units.

### Histopathological Analysis

Tissue samples were fixed in 10% neutral buffered formalin (Azer Scientific, Morgantown, Pa.), paraffin embedded and sectioned. H&E and Masson's trichrome staining were performed by means of standard protocols. Immunohistochemistry (IHC) for FAH was performed.

### Statistical Analysis

Numerical data are expressed as mean (±standard deviation). Mann-Whitney U test was used to analyze differences in continuous variables between spheroid and single cell groups. Statistical analyses were performed with GraphPad Prism software version 7. Statistical significance was established when p<0.05.

### Results

Spheroids Show a Higher Efficiency of Vector Transduction than Single Cell Hepatocytes

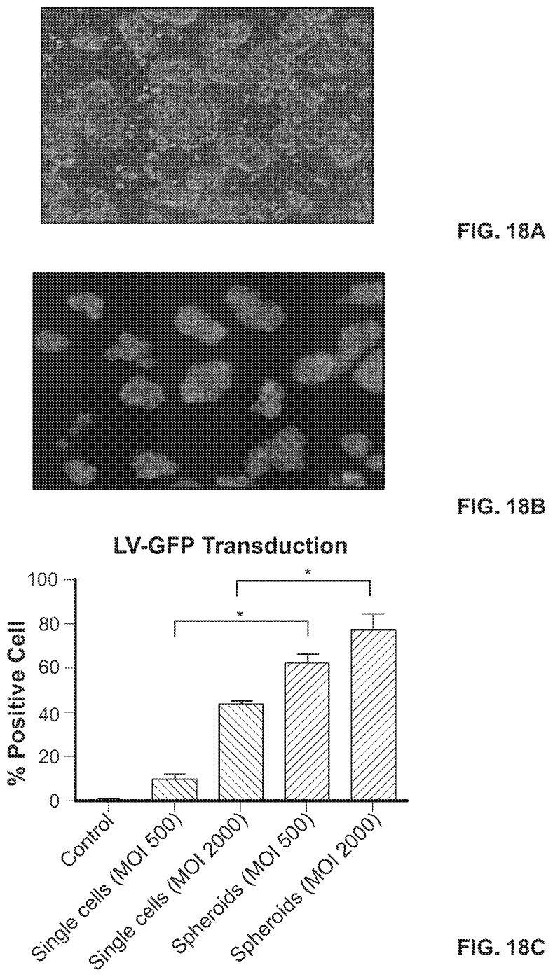

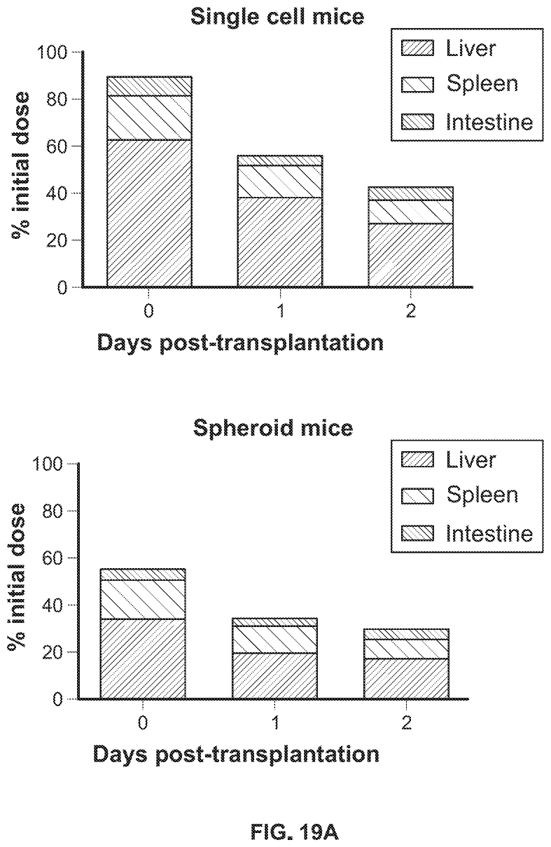

To evaluate transduction efficiencies in spheroid hepatocytes compared to single cell hepatocytes, an in vitro experiment was performed where primary pig hepatocytes in both forms were transduced with a lentiviral vector expressing GFP at different MOIs. Percentage of GFP-positive cells detected by flow cytometry 96 h after transduction was significantly higher in spheroid than in single cell groups (FIGS. 18A to 18C).

### Hepatocyte Spheroids Engraft in the Liver in Mice

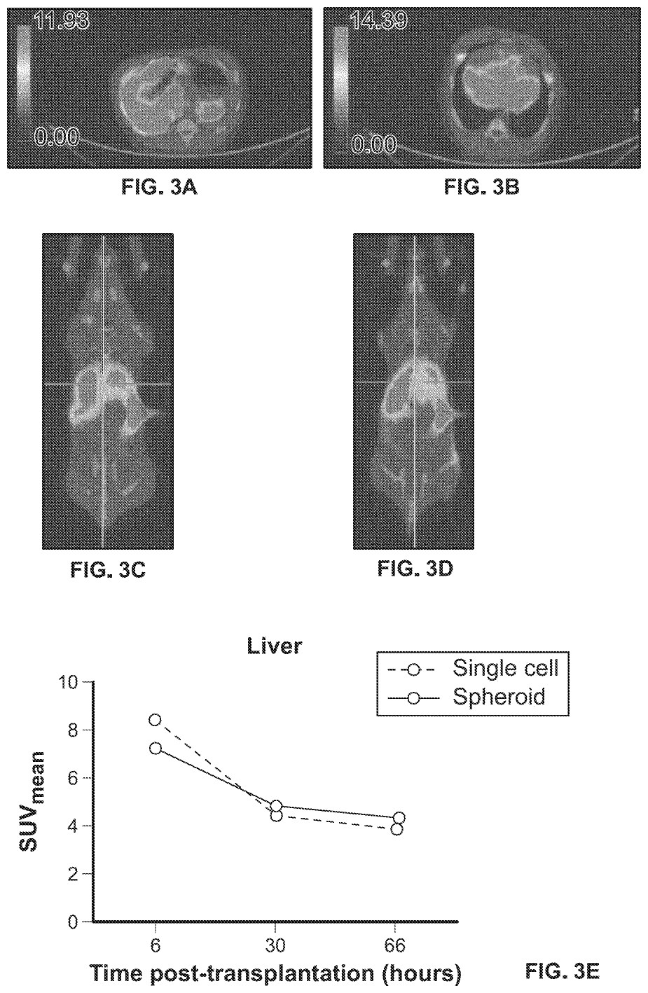

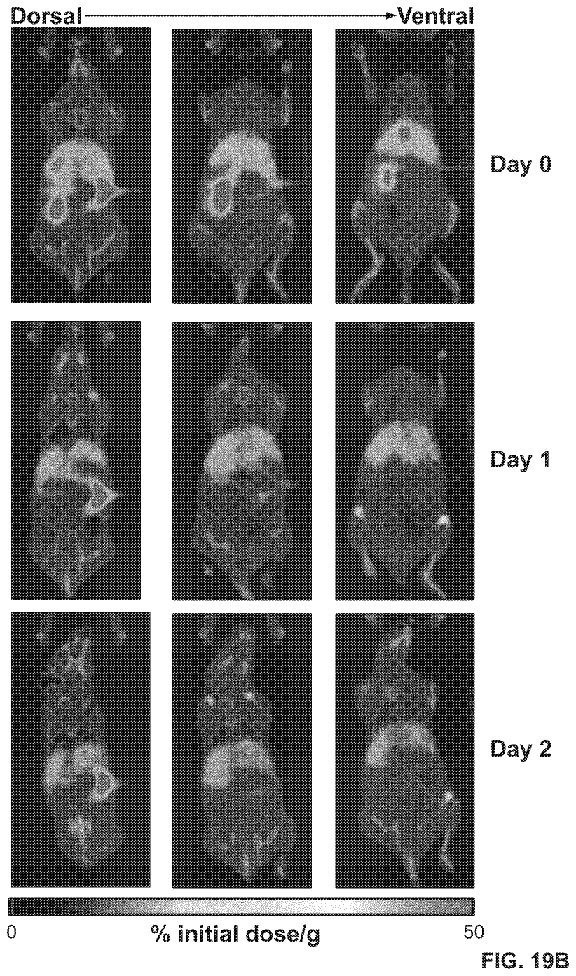

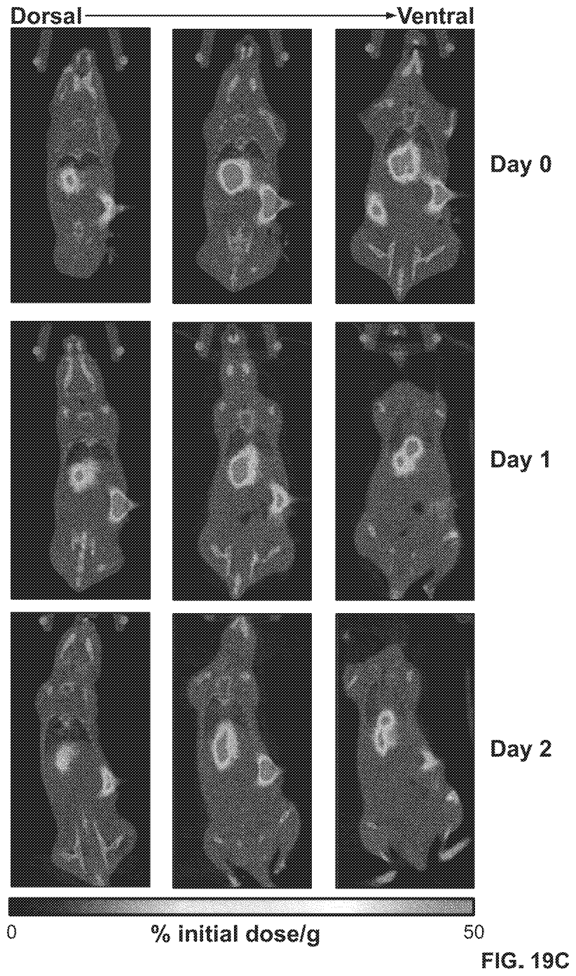

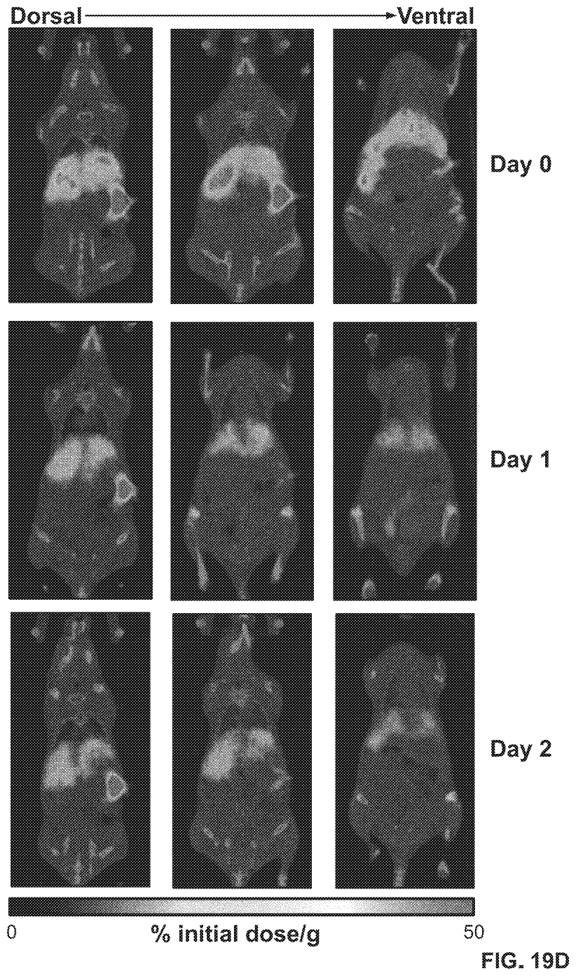

To compare engraftment of spheroid hepatocytes to single cell hepatocytes, SCID beige mice were randomized to receive intrasplenic injection of single cell hepatocyte suspension (n=4) or spheroid hepatocyte suspension (n=4). Hepatocytes were obtained from a wild-type pig harvest and were radiolabeled with ⁸⁹Zr (half-life 78.4 h). The radiolabeling efficiency was ˜20%. The radioactivity concentration in both single cell and spheroid forms was ˜0.1 MBq/10⁶ cells. Each mouse was injected with approximately 665,000 cells. MicroPET-CT imaging at 2, 24, and 48 h post-transplantation showed migration and engraftment in the liver of a majority of intrasplenically-injected cells in both single cell and spheroid groups. However, it also showed a higher percentage of the initial dose remaining in the liver in the single cell mice compared to spheroid mice (FIG. 19A), which approached significance (p=0.06). Other main sites of biodistribution were spleen and, to a lesser degree, intestine. In addition to higher liver positivity in the single cell group, biodistribution of cells within the liver was visibly different between single cell (FIG. 3C; FIG. 19B) and spheroid (FIG. 19C) mice. Inter-mouse variability was higher in the spheroid group, with a more heterogeneous distribution of cells when compared to single cell mice FIGS. 19C and 19D).

### Hepatocyte Spheroids Engraft in the Liver in Pigs

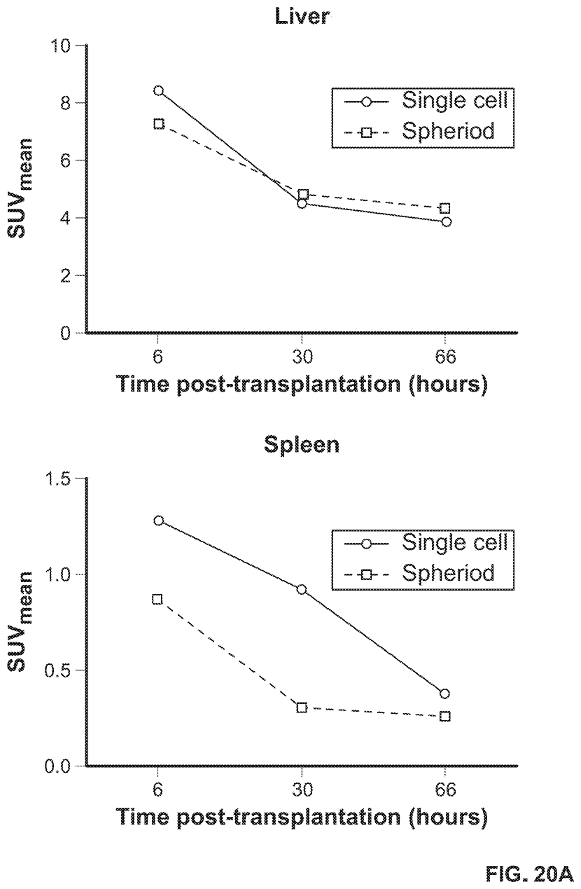

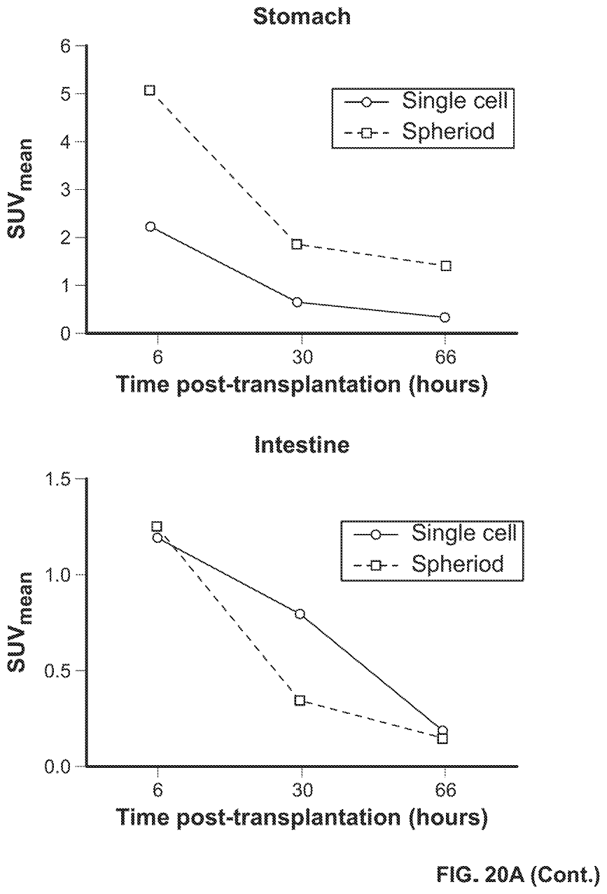

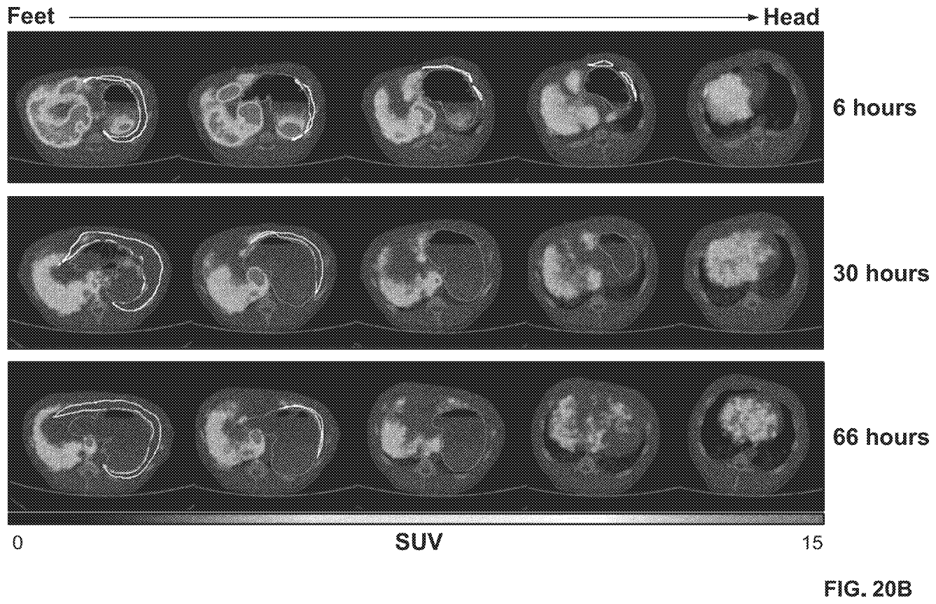

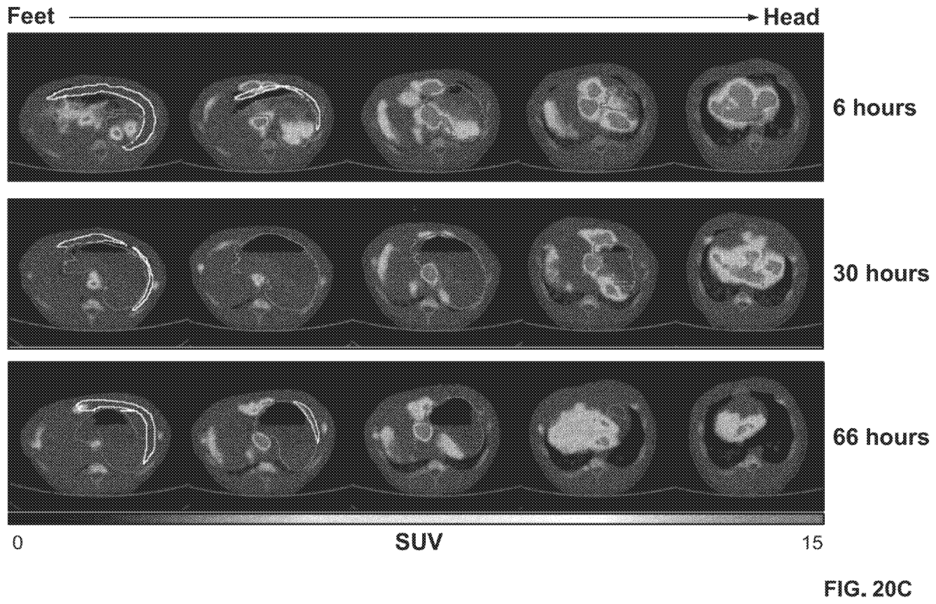

Spheroid hepatocyte transplantation was then compared to single cell transplantation in wild-type pigs. After laparoscopic liver resection and ex vivo hepatocyte isolation, autologous hepatocytes were transplanted as single cell suspension (n=1) or spheroid suspension (n=1) using ultrasound-guided percutaneous portal vein infusion. Each pig was injected with 4.18×10⁷ radiolabeled cells (˜0.1 MBq/10⁶ cells, 8.36×10⁵ cells/mL) in saline. No complication was noted in either procedure. PET-CT imaging at 6, 30, and 66 h post-transplantation showed no difference in total liver radioactivity levels in the liver (8.50, 4.56, 3.91 vs. 7.34, 4.90, 4.39 SUV; p>0.99), spleen (1.29, 0.93, 0.39 vs. 0.87, 0.32, 0.27 SUV; p=0.25), stomach (2.20, 0.64, 0.32 vs. 5.03, 1.82, 1.40 SUV; p=0.75), or intestine (1.19, 0.79, 0.19 vs. 1.24, 0.35, 0.15 SUV; p=0.25) between the single cell and spheroid animals (FIG. 20A). There was no evidence of radiotracer present in the lungs or other organs. In both pigs, cells were efficiently cleared out of spleen and stomach, with significant levels of radioactivity remaining only in the liver at 66 h. However, PET-CT shows significant heterogeneity in the distribution pattern of the spheroid hepatocytes when compared with the single cell suspension animal (FIGS. 3A and 3B; FIGS. 20B and 20C). Axial PET-CT images from the spheroid transplant demonstrate a high signal intensity in the central segments of the liver with minimal distribution in the lateral segments. In contrast, distribution of single cell hepatocytes appears relatively homogeneous within the liver.

Hepatocyte Spheroids are Able to Engraft and Expand in a Pig Model of Human HT1 after Ex Vivo Lentiviral Gene Transfer

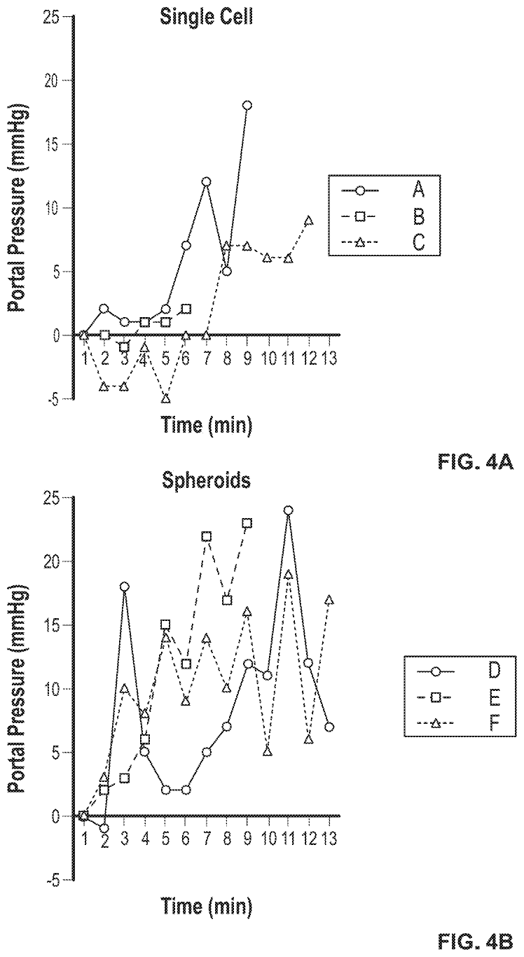

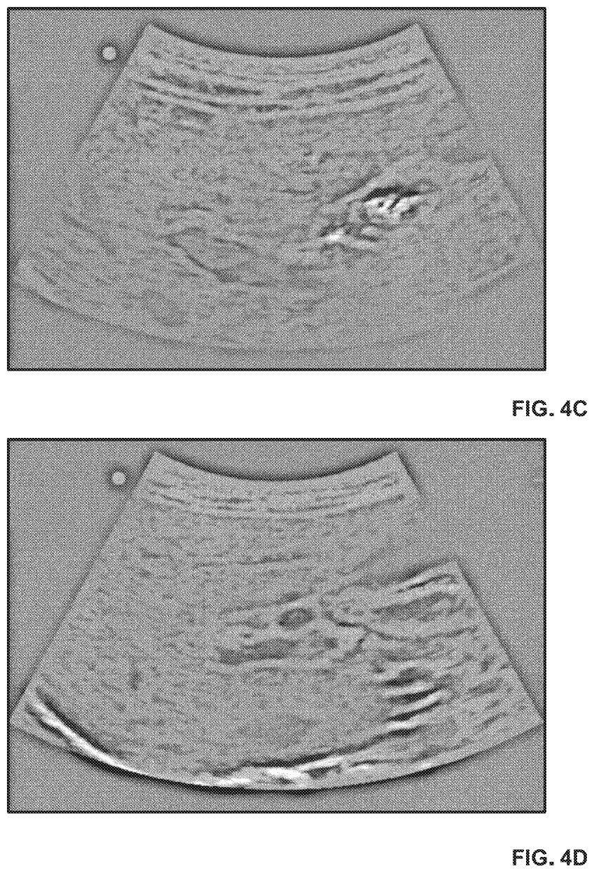

Autologous transplantation of spheroid hepatocytes was compared to single cell suspension after ex vivo gene delivery of the porcine Fah gene in 6 Fah^{-/-} pigs. Harvest results and dose of cells injected into each pig are shown in Table 1. Pooled mean diameter of transplanted spheroids was 76.65±18.97 μm. Portal pressures were monitored during intraportal infusion: single cell transplanted animals experienced a mean change in portal pressure of 2.53±2.51 mmHg and animals transplanted with spheroid hepatocytes experienced a significantly higher mean change in portal pressure of 10.98±0.31 mmHg (p<0.01; FIG. 4A). In all spheroid animals, injections were discontinued due to rises in portal pressure. Furthermore, two spheroid pigs developed portal vein thrombi (FIG. 4B). These animals were treated with enoxaparin at the therapeutic dose of 1 mg/kg twice daily for seven days, at which point ultrasound showed complete resolution of the thrombi. In the third spheroid animal, systemic anticoagulation was performed with an 80 U/kg bolus of intravenous heparin immediately prior to transplantation, and no significant thrombotic complications were noted but significant changes in portal venous pressures were observed once again.

TABLE 1
Summary of cell transplantations.

| | | Weight | Cells | | Cells | | Cells |
|---|---|---|---|---|---|---|---|
| Pig | Single cell vs. | at transplant | harvested | | transplanted | Cells transplanted | transplanted |
| ID | spheroid | (kg) | (g) | Viability (%) | (g) | (×10⁶) | (×10⁶/kg) |
| 889 | Single cell | 16.4 | 7.4 | 89 | 7.4 | 720 | 44 |
| 896 | Single cell | 18.2 | 4.8 | 52 | 4.8 | 250 | 14 |
| 960 | Single cell | 10.6 | 14.4 | 97 | 11.6 | 2,250 | 212 |
| Single cell mean ± SD | | 15.1 ± 4.0 | 8.9 ± 5.0 | 79.3 ± 24.0 | 7.9 ± 3.4 | 1,100 ± 1,000 | 90 ± 110 |
| 888 | Spheroid | 17.4 | 23.9 | 90 | 10.8 | 1,870 | 107 |
| 897 | Spheroid | 16.4 | 19.3 | 92 | 9.1 | 1,960 | 120 |
| 958 | Spheroid | 16.4 | 12.8 | 93 | 10.1 | 1,340 | 82 |

| | | | | | | |
|---|---|---|---|---|---|---|
| Spheroid mean ± SD | 16.7 ± 0.6 | 18.7 ± 5.6 | 91.7 ± 1.5 | 10.0 ± 0.9 | 1,700 ± 300 | 100 ± 20 |

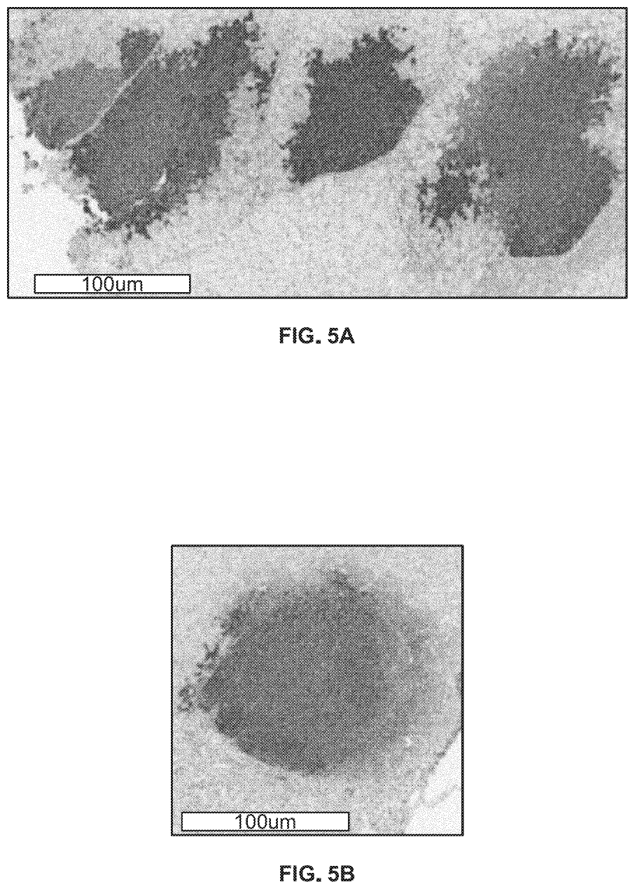

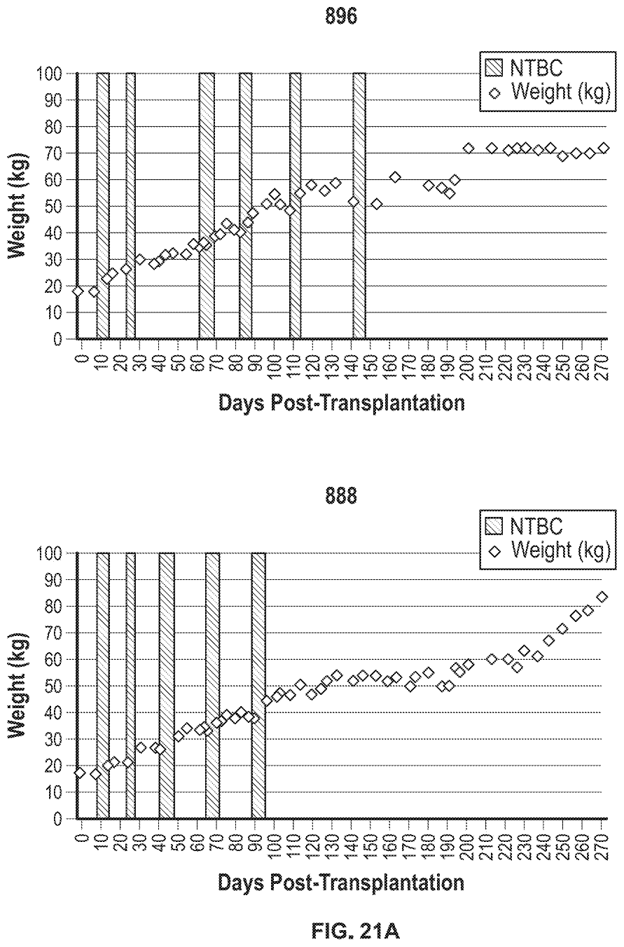

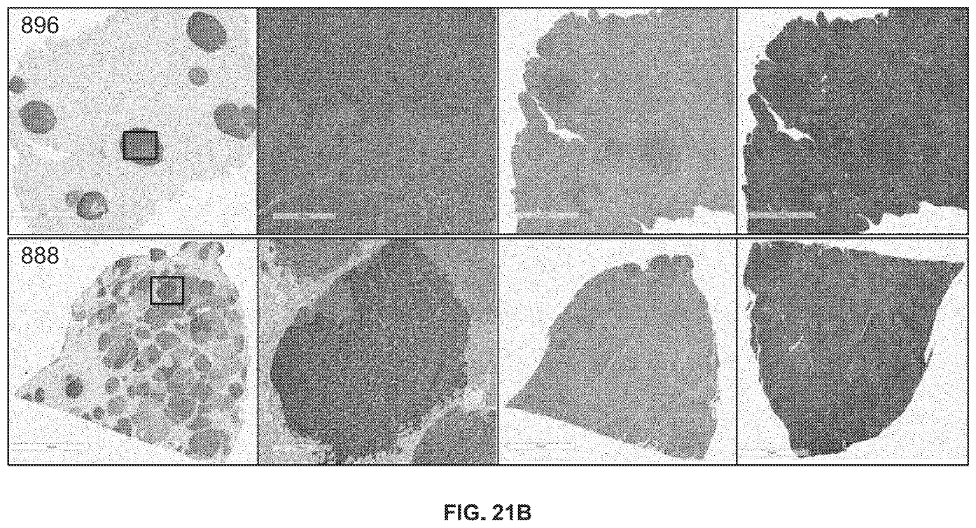

All animals remained on NTBC until the time of transplantation, at which point NTBC administration was discontinued to stimulate expansion of the transplanted FAH-positive hepatocytes. Animals were then cycled on and off NTBC based on weight and clinical parameters until weight stabilization occurred (FIG. 21A). Only one single cell animal (896) was kept as a control for the spheroid group. At 6 months post-transplantation, laparoscopic liver biopsies were performed on one single cell (896) and one spheroid (888) animal. IHC showed multiple FAH-positive nodules within all three remaining liver lobes in both pigs, demonstrating expansion clusters of engrafted cells. At 9-10 months post-transplantation, all animals were euthanized. Terminal histology showed robust FAH expression in all pigs, and no evidence of any major pathology by H&E staining (FIG. 5A (spheroid); FIG. 5B (single cell); FIG. 21B).

### Discussion

Primary hepatocytes are difficult to maintain in vitro due to dedifferentiation and loss of proliferative capacity. Therefore, the clinical use of hepatocyte transplantation is still significantly limited by loss of viability and metabolic function in these cells. Spheroids, three-dimensional hepatocyte aggregates, may circumvent some of these issues due to their improved longevity and phenotypic durability when compared to single cell hepatocytes. Spheroid-like structures have been used experimentally to enhance cell transplantation in a number of studies. Hepatocyte spheroids were created through a different method and transfected with non-viral vectors to show that subcutaneous transplantation of these spheroids resulted in longer term transgene expression and preservation of proper functionality. These data, together with the higher lentiviral vector transduction efficiency we show in our rocker-formed spheroids as compared to single cell hepatocytes, make spheroids useful for intraportal hepatocyte transplantation worth further development.

Our biodistribution studies showed initial presence of both single cell hepatocytes and hepatocyte spheroids in liver, spleen, and digestive tract, with cells being progressively cleared out of spleen and digestive tract until only the liver remained positive. Biodistribution of single cell hepatocytes transplanted through portal vein infusion has been evaluated in humans with pediatric ornithine transcarbamoylase deficiency patient. In this study, a predominant hepatic distribution with an average liver-to-spleen ratio of 9.5 to 1 and no significant pulmonary radiotracer activity was found. Our results, obtained using a novel radiotracer and high-resolution imaging technology, are consistent with these data in both single cell and spheroid pigs. Pulmonary translocation of hepatocytes after transplantation has been described in several studies, but we found no evidence of radiotracer in the pulmonary fields, possibly due to the careful monitoring and prevention of portal hypertension in our models.

Although there was no significant difference in overall biodistribution in terms of total cells in the liver between single cell hepatocytes and hepatocyte spheroids, we found dissimilarities in distribution of cells within the liver between the two groups, with spheroids presenting a more uneven, irregular pattern. Heterogeneity in distribution of intraportally transplanted single cell hepatocytes within the liver has been previously described. In this previous study, heterogeneity was due to differential intrahepatic portal venous blood flow. Heterogeneity observed in our study is likely due to spheroid size where steric restrictions through the distal portal ramifications might limit transplant engraftment.

The thrombogenic potential of spheroids presents an important safety issue. It has been shown in pigs that portal pressures increase linearly with cell load, and that even with single cell hepatocyte infusion thrombi are formed in segmental portal branches. These concerns are magnified with the use of spheroids. In our study, portal pressures during infusion were significantly higher with spheroid than with single cell suspensions, and ⅔ of the spheroid-transplanted animals developed thrombotic complications requiring systemic anticoagulation. Safety issues, however, could be overcome by modification of infusion parameters and anticoagulation protocols. Use of a gravity-fed bag system instead of the syringe method could allow for a controlled rate of infusion through a natural reduction in flow following any increase in portal pressure. Prophylactic systemic anticoagulation in our third pig appeared to prevent thrombus formation during spheroid infusion, although any interpretation must be contextualized as a single observation. The Edmonton Protocol for islet transplantation in humans does include a therapeutic heparin infusion, as well as low molecular weight heparin for a week after transplantation. Even so, partial-branch venous occlusion does occasionally occur and is managed with further anticoagulation and Doppler ultrasound follow-up without any further complications, as was the case in our study.

Despite safety issues and differences in distribution between single cell hepatocytes and spheroid hepatocytes, we have demonstrated that spheroid transplantation with hepatocytes after ex vivo gene therapy is able to effectively correct the metabolic deficiency in the clinically relevant pig model of HT1. Spheroids demonstrated the ability to engraft and proliferate in, and eventually repopulate Fah^{-/-} livers, making this method a viable alternative to single cell hepatocyte transplantation for the treatment of metabolic liver disease. Furthermore, conclusions drawn from this study are applicable to allogeneic hepatocyte transplantation for the treatment of other liver diseases.

□□hepatocytes to be a relevant alternative to single cell hepatocytes for transplantation after ex vivo gene therapy. Hepatocytes cultured to generate spheroids demonstrated a significantly higher efficiency of vector transduction and a comparable overall volume of cells within the liver after transplantation, despite showing a more heterogeneous biodistribution than single cell hepatocytes. Importantly, spheroid transplantation resulted in successful engraftment and in vivo expansion of corrected autologous hepatocytes, and treatment of the clinically relevant large animal model of human HT1.

### Example 4

A porcine model of hereditary tyrosinemia type 1 (HT1) was treated with in vivo liver-directed lentiviral vector gene therapy.

### Methods

### Lentiviral Vector

A lentiviral vector was constructed as described in Example 2.

### Administration

Direct percutaneous portal vein infusions of a lentiviral vector carrying the human Fah gene under control of the hepatocyte-specific alpha1-antitrypsin promoter at a dose of 2×10¹⁰ transfection units (TU)/kg was conducted in four fumarylacetoacetate hydrolase (FAH)-deficient pigs at six weeks of age. Treated animals were cycled on and off with 25 mg NTBC/nitisone administered orally in the chow. Metabolic correction was followed through biochemical analysis of liver function and tyrosine levels, histological analyses of liver fibrosis and FAH-positive hepatocytes, and clinically through the animal's ability to thrive off the protective drug NTBC/nitisinone.

### PCR Analysis of Integration

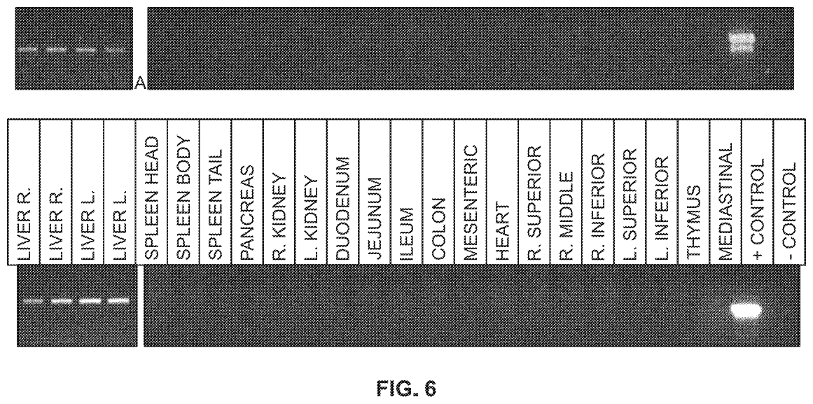

Integration profile of the lentiviral vector was studied through PCR analysis of all major intraabdominal and thoracic organs. The sequences and location of the PCR primers are shown in FIG. 14 and FIG. 15. Tissues and organs included: liver, spleen, pancreas, kidney, duodenum, jejunum, ileum, colon, mesenteric lymph node, heart, lung, thymus, and mediastinal lymph node. Three primer pairs were used to analyze for presence of LV-hFAH in multiple samples of thirteen different tissue types at 48 hours (Betsy/pig1, top) and 60 days (Donald/pig2, bottom) post-treatment. (FIG. 6)

### Histology

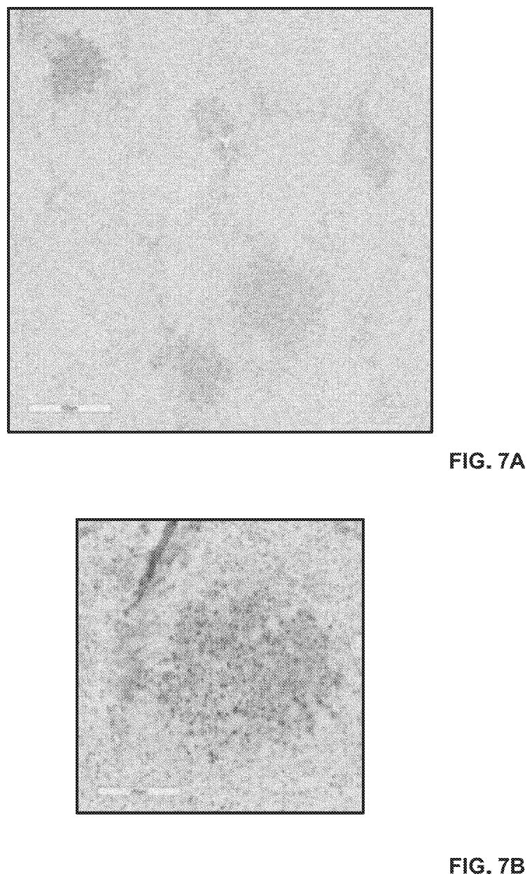

Immunohistochemical analysis of FAH-expression in hepatocytes was conducted using standard methods. For tissue analysis, samples were fixed in 10% neutral buffered formalin (PROTOCOL, Fisher Scientific) and processed for paraffin embedding and sectioning. For hematoxylin and eosin staining slides were prepared using standard protocols (FIGS. 7A and 7B).

### Biochemical Characterization

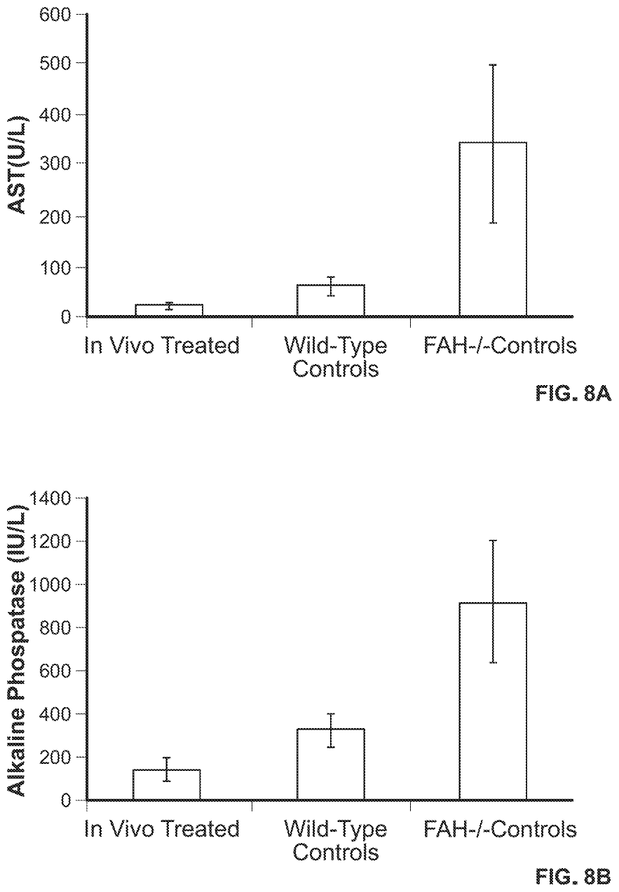

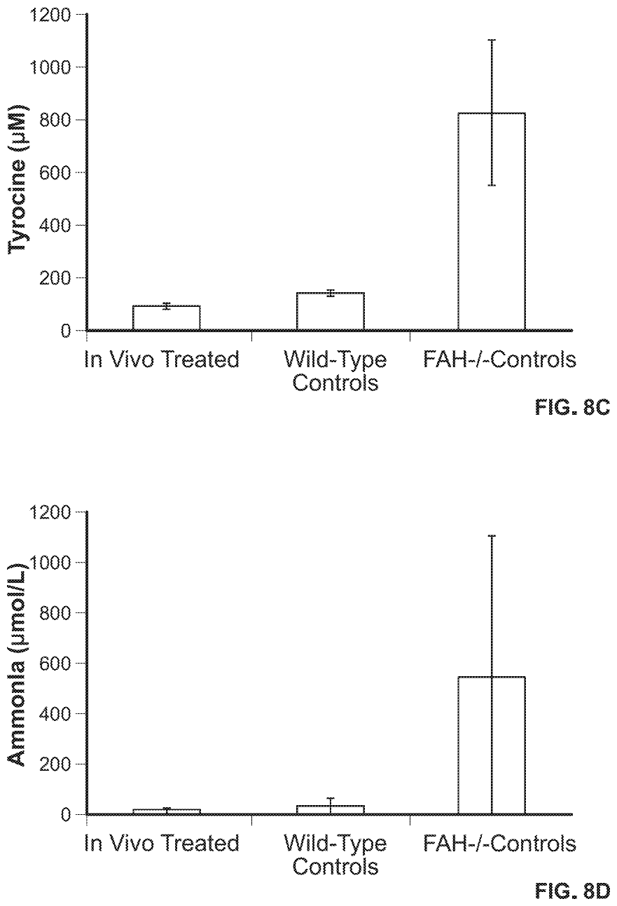

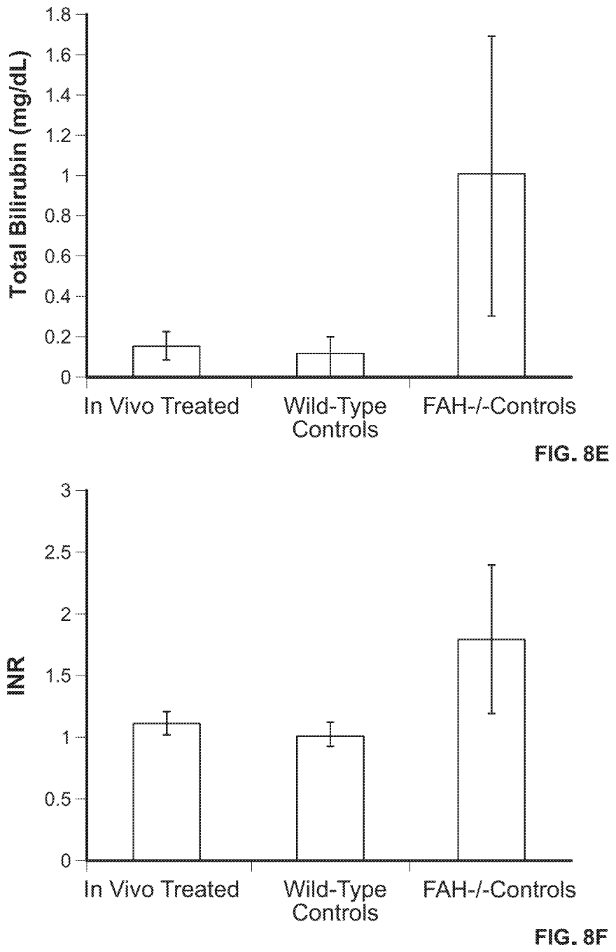

Metabolic correction was followed through biochemical analysis of liver function and tyrosine levels, histological analyses of liver fibrosis and clinically through the animal's ability to thrive off the protective drug NTBC/nitisinone. The levels of aspartate amino transferase (AST), alkaline phosphatase, tyrosine, ammonia, total bilirubin, and international normalized ratio (INR) were measured in in vivo treated animals, wild type control animals, and FAH−/− animals at 5 months post treatment (FIG. 8).

### Weight Stabilization

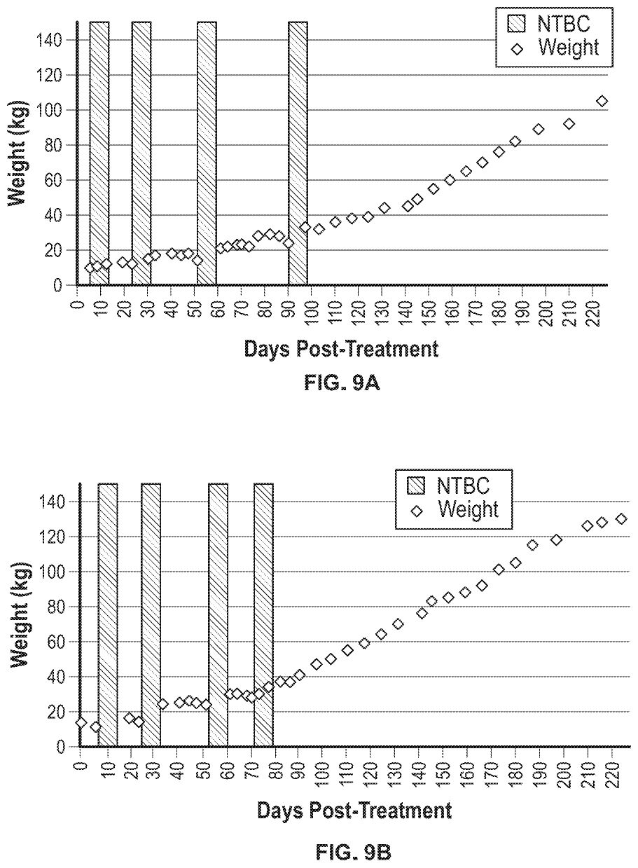

Weights of animals were measured in kg over time and before and after NTBC withdrawal (FIGS. 9A and 9B).

### Results and Conclusions

One animal developed an acute inflammatory reaction and died in the 48 hours following treatment. Three animals were cycled on and off NTBC to stimulate expansion of FAH-positive hepatocytes.

Terminal end points were at 48 h, 2 months, and 1 year. At two months post-treatment, liver harvest in one pig showed presence of multiple FAH-positive hepatocyte nodules with no evidence of fibrosis as shown in FIGS. 7A and 7B. Three animals were cycled with NTBC to stimulate expansion of FAH-positive hepatocytes. Two months post-treatment, liver harvest showed >10% FAH-positive hepatocytes within the liver. Histology showed 100% complete liver repopulation with FAH-positive hepatocytes, no significant fibrosis and no HCC.

Biochemistry showed normalization of tyrosine levels and liver function tests at 6 months. Preliminary PCR analysis demonstrated lentiviral vector integration exclusively in the liver, with no evidence of lentiviral vector DNA in any other major organ as shown in FIG. 6. Next generation sequencing and bioinformatics in hepatocytes revealed a benign integration profile with no preference for tumorigenic genes or promoter regions.

Portal vein delivery may preclude systemic dissemination of the lentiviral vector at appropriate therapeutic titers. In vivo gene transfers can effectively correct the metabolic deficiency in FAH-deficient pigs within two to three months of treatment, with no detectable lentiviral integration in non-hepatic tissues. As shown in FIG. 8A-E, the levels of AST, alkaline phosphatase, tyrosine, ammonia, total bilirubin, and international normalized ratio (INR) were more similar to the levels in wild type controls than the FAH−/− pig when measured 5 months after infection. The two remaining animals became NTBC-independent 78 and 98 days post-treatment, and are currently gaining weight age-appropriately off NTBC (FIGS. 9A and 9B). Blood chemistry shows complete normalization of tyrosine levels. These results show that in vivo gene transfers can effectively correct the metabolic deficiency in FAH-deficient pigs with no evidence of significant lentiviral integration in non-hepatic tissues.

### Example 5

### Introduction

The effectiveness of cell-based therapeutics to treat liver failure is limited by the inflammatory, fibrotic environment of the diseased liver. Alternative anatomical sites for transplantation of corrected cells could provide a healthier milieu to enable hepatocyte engraftment and proliferation. In this study, we examined ectopic transplantation of ex vivo corrected hepatocytes into lymph nodes in the pig model of HT1, and their role in treating acute liver failure.

### Materials and Methods

### Animals

All animal procedures were performed in compliance with Mayo Clinic's Institutional Animal Care and Use Committee regulations and all animals received humane care. For biodistribution experiments, a female heterozygous Fah^{+/-} pig was used. For ex vivo gene therapy experiments, male and female Fah^{-/-} pigs were used. Fah^{-/-} pigs were produced in a 50% Large White and 50% Landrace pig as previously described.

### NTBC Administration

NTBC mixed in food was administered at a dose of 1 mg/kg/day with a maximum of 25 mg/day. All animals remained on NTBC until the time of transplantation, after which NTBC administration was discontinued to support expansion of the corrected cells. After hepatocyte transplantation, all animals were monitored daily for loss of appetite or any other clinical signs of morbidity. Animals were weighed daily for the first two weeks post-operatively and weekly thereafter. If loss of appetite, weight loss, or any other signs of morbidity occurred, NTBC treatment was reinitiated for seven days. Animals were cycled on and off NTBC in this fashion to stimulate expansion of corrected FAH-positive cells.

### Liver Resection and Hepatocyte Isolation

Eight-week-old (16-21 kg) pigs underwent a laparoscopic partial hepatectomy involving the left lateral lobe under inhaled general anesthesia with 1-3% isoflurane. Resection volumes represented 15 to 20% of the total liver mass. Using an open Hasson Technique, a 12 mm port was placed into the peritoneal cavity and the abdomen insufflated. Using a 5 mm laparoscope (Stryker, Kalamazoo, Mich.) two additional 5 mm ports were placed under direct visualization. The left lateral segment and its vascular and biliary drainage was isolated and divided using an Endo GIA stapler (Covidien, Dublin, Ireland). The liver section was retrieved using an Endo Catch bag (Covidien, Dublin, Ireland), and adequate homeostasis was ensured prior to port removal and incision closure. This liver section was then perfused ex vivo through the segmental portal vein with a two-step perfusion system to isolate hepatocytes as previously described Number and viability of cells were determined by trypan blue exclusion.

### Hepatocyte Radiolabeling

For early biodistribution experiments, hepatocytes were radiolabeled in suspension with ⁸⁹Zr with synthon ⁸⁹Zr-DBN at 27° C. for 45 min in Hanks Buffered Salt Solution as previously described.

### Hepatocyte Transduction

Hepatocytes were co-transduced in suspension at a MOI of 20 TUs with lentiviral vectors carrying the sodium-iodide symporter (NIS) reporter gene or the pig FAH gene or under control of the thyroxine-binding globulin promoter. The NIS reporter allows for longitudinal non-invasive monitoring of transplanted cells through single-photon emission computed tomography (SPECT) or positron emission tomography (PET) Transduction occurred over the course of two hours before transplantation using medium and resuspension techniques previously described Hepatocytes were resuspended in 0.9% sodium chloride (Baxter Healthcare Corporation, Deerfield, Ill.).

### Hepatocyte Transplantation in Pigs

All pigs received autologous transplantation of hepatocytes through direct mesenteric lymph node injection. After their partial hepatectomy, animals were kept under general anesthesia until the time of transplantation, approximately 4 h later. Bowel was exteriorized through the upper midline incision until the root of the mesentery was visible. Mesenteric lymph nodes were identified and hepatocytes were delivered directly into 15-25 nodes through direct injection with a 25-gauge one-inch needle. Each animal received a total of 6×10⁸ hepatocytes. Heparinization of the cell solution just prior to injection was performed at 70 U/kg of recipient weight as has been previously described for islet cell transplantation protocols.

### Positron Emission Tomography-Computed Tomography (PET-CT) Imaging and Analysis

Imaging was performed on the high-resolution GE Discovery 690 ADC PET/CT System (GE Healthcare, Chicago, Ill.) at 6, 54, and 150 h post-transplantation in the ⁸⁹Zr-labeled animal, and at 3, 5, and 6 mo post-transplantation in two of the NIS-labeled animals. CT was performed at 120 kV and 150 mA, with tube rotation of 0.5 s and pitch of 0.516. PET was performed as a two-bed acquisition with 10 min per bed and 17-slice overlap, resulting in a 27-cm axial field of view. PMOD (version 3.711; PMOD Technologies, Switzerland) was used for image processing, 3D visualization and analysis. The anatomic location of liver and kidneys were identified from the registered datasets. Volumes of interest (VOIs) of mesenchymal lymph nodes were created on PET images, and standardized uptake values (SUVs) for body weight were obtained. Surface rendering was performed using the threshold pixel value for bone, liver and kidneys from the CT dataset and lymph nodes from the PET dataset to localize the lymph nodes with reference to the skeleton.

### Biochemical Analysis

Standard serum and plasma analyses were performed. Tyrosine values in plasma were determined using liquid chromatography and tandem mass spectrometry via Mayo Clinic's internal biochemical PKU test.

### Histopathological Analysis

For H&E and Masson's Trichrome staining, as well as FAH immunohistochemistry (IHC), tissue samples were fixed in 10% neutral buffered formalin (Azer Scientific, Morgantown, Pa.), paraffin embedded and sectioned (5 rpm). H&E and Masson's trichrome staining were performed by means of standard protocols.

### Immunohistochemistry

Immunohistochemistry for FAH was performed as previously described Percent of hepatocytes positive for FAH staining was quantified using a cytoplasmic stain algorithm in Aperio ImageScope. For all other immunohistochemical stains, tissue was fixed in 4% paraformaldehyde for 4 h, maintained in 30% sucrose for 12 h and then embedded in OCT medium, frozen, and stored at −80° C. Sections were washed with PBS and blocked with 5% BSA or milk for 30 min. Sections were then incubated with primary antibody for 1 h and secondary antibody for 30 min, and were mounted with Hoechst mounting media. Images were captured with an Olympus IX71 inverted microscope. The following reagents were purchased for immunohistochemistry: ER-TR7 (Abcam ab51824 1:500), Glutamine Synthetase (Abcam ab49873 1:500 and ab64613 1:200), CD31 (BIO-RAD MCA1746GA 1:100), Cytokeratin 7 (Abcam ab9021 1:100) and Cytokeratin 18 (Proteintech 10830-1-AP 1:100 and LifeSpan BioSciences LS-B11232 1:200).

### Next Generation Sequencing

Genomic DNA was isolated from tissues using a Gentra Puregene Tissue Kit (Qiagen, Hilden, Germany). Ligation-mediated PCR (LM-PCR) was used for efficient isolation of integration sites. Restriction enzyme digestions with MseI were performed on genomic DNA samples; the digested DNA samples were then ligated to linkers and treated with ApoI to limit amplification of the internal vector fragment downstream of the 5′ LTR. Samples were amplified by nested PCR and sequenced using the Illumina HiSeq 2500 Next-Generation Sequencing System (San Diego, Calif.). Integration sites were judged to be authentic if the sequences began within 3 base pairs of the lentiviral 3′ LTR end, showed a >98% sequence match, and yielded a unique best hit when aligned to the pig genome using the Burrows-Wheeler Alignment algorithm (BWA-MEM).

### Bioinformatics Analysis

Quality control of the sequencing reads was performed with FASTQC. The reads were then trimmed in two separate steps: 1) viral sequence trimming: the viral sequence was trimmed using Cutadapt with a mismatch rate (e=0.3) from the 5′ end of first read (R1) and the 3′end of the second read (R2) of the sequenced read pair; 2) linker trimming: the linker sequence was trimmed using Cutadapt from the 3′end of R1 and the 5′ end of R2. After each trimming step, reads that became too short (<15 bp) were filtered out. After trimming, the reads were mapped to the susScr11 build of the pig reference genome using BWA-MEM in single-end mode. The single-end mode was used instead of paired-end for mapping as the insert size of read pairs was determined to be too short, and resulted in overlapping mates. Default BWA-MEM parameters were used for the mapping process. Additionally, reads were also checked for contamination from potential bacterial, viral and model organism sources other than lentivirus by randomly selecting one million unmapped reads after alignment and using BLAST for querying these reads against NCBI's Genbank database.

The aligned reads were subjected to multiple filtering criteria to be selected for viral integration point identification. A site of integration is defined by the position on the pig genome of the first base immediately following the viral sequence that was trimmed by Cutadapt. To be selected for integration point identification, the reads needed to: 1) have been successfully trimmed with the viral sequence, yielding a read length shorter than the original read length (<150 bp); and 2) map uniquely to the genome with a BWA-MEM mapping quality score of greater than zero. Since the first read (R1) of a sequenced read pair predominantly contained the trimmed viral sequence, and the second read (R2) largely overlapped the first read, we excluded the second read from the analysis as it did not provide any additional value for detecting integration points. Using this approach, unique integration points were identified across the genome without any constraints on coverage. However, for downstream annotation and analysis, only those integration points which had a total read coverage of at least 5× were used to minimize false positives.

The integration points were then extensively annotated using in-house Python scripts with gene and feature information using the susScr11 Refflat file of the pig genome obtained from the UCSC Table Browser. The annotations included categorizing the integration points into those falling in genomic features such as exons, introns, 3 prime UTRs, 5 prime UTRs and intergenic regions. To avoid conflict of feature categorization arising from multiple overlapping transcript definitions of the same gene, only the longest transcript of each gene was used for the annotation process. Additionally, the distance of each integration point from the transcription start site (TSS) of the nearest gene was calculated. The integration points were further annotated to assess their presence or absence in CpG-rich regions of the genome (“CpG islands”) using the susScr11 CpG island definitions obtained from the UCSC Table Browser.

Additionally, we also used an in-house Mayo Large NGS Tumor Panel, which contains targeted regions from approximately 745 human genes to assess the distribution and density of the integration points in the pigs within these tumor-associated genes. This was done using the assumption that the tumor-associated genes are generally functionally homologous between humans and pigs and any viral integration in these tumor-associated genes in the pig would subsequently result in similar gene expression profile in the pigs as that found in humans.

### RNA Isolation, Pre-Screening and Sequencing

Snap-frozen tissue samples of hepatized or control (non-transplanted) mesenteric lymph nodes from three experimental animals, and of engrafted (from three experimental animals) or control livers (from one control animal) were shipped on dry ice for RNA isolation and pre-screening prior to sequencing. Briefly, RNA isolation was performed using the RNeasy Mini Kit (Qiagen) according to manufacturer's instructions (including the optional on-column DNase digestion). RNA purity and concentration were measured using a NanoDrop 2000/c spectrophotometer (Thermo Fisher Scientific). Pre-screening of hepatized lymph nodes was performed by examining Albumin and FAH expressions using quantitative reverse transcription polymerase chain reaction (qRT-PCR). Non-transplanted mesenteric lymph nodes and control livers were used as negative and positive controls, respectively. RNA was retrotranscribed using the iScript Reverse Transcription Supermix (Bio-Rad) and Albumin and FAH amplified using the SsoAdvanced™ Universal SYBR® Green Supermix (Bio-Rad) on a StepOnePlus Real-Time PCR System (Applied Biosystems). Expression of glyceraldehyde-3-phosphate dehydrogenase (GAPDH) was used for normalization of gene expression data. Relative changes in gene expression were calculated using the 2^{-ΔΔCT} method. RNAs from selected samples were shipped on dry ice to Novogene in Sacramento, Calif. for library preparation and sequencing. All samples passed Novogene internal quality control with RNA integrity number above eight.

### RNA Sequencing Data Analysis

Pre-processing of RNAseq data was performed by a standardized and reproducible pipeline and were analyzed using Partek Genomics Suite software version 7.0. Briefly, quality control was measured taking into account sequence-read lengths and base-coverage, nucleotide contributions and base ambiguities, quality scores as emitted by the base caller and over-represented sequences. All the samples analyzed passed all the QC parameters and were mapped to the annotated pig reference SScrofa11.1_90+nonchromosomal using STAR2.4. id index standard settings.

Estimation of transcript abundance based on the aligned reads was performed by optimization of an expectation-maximization algorithm (strict pair-end compatibility and introns matching by junction reads) and total reads in a sample were divided by one million to create a per million scaling factor for each sample; then the read counts were divided by the per million scaling factor to normalize for sequencing depth and give a reads per million value; and finally divided per million values by the length of the gene in kilobases to normalize for size (RPKM).

Differentially expressed genes were detected using differential gene expression (GSA) algorithm based on p-value of the best model for the given gene, false discovery rate (i.e. fraction of the false positive results) based on step-up method by Benjamini and Hochberg (FDR), ratio of the expression values between the contrasted group linear, fold change between the groups and least-square means (adjusted to statistical model) of normalized gene counts per group. After filtering the data comprised 12,000 present genes (P-value with FDR of 0.05, fold change of 121). Unsupervised clustering to visualize expression signature was performed using 1-Pearson correlation distance and complete linkage rule and samples were classified using t-SNE (t-distributed stochastic neighbor embedding).

### Statistical Analysis

Numerical data are expressed as mean (±standard deviation). Statistical significance was determined by Welch's t-test, and established when p<0.05. Statistical analyses were performed with GraphPad Prism software version 7.

### Results

Hepatocytes Engraft in the Mesenteric Lymph Nodes after Ectopic Transplantation

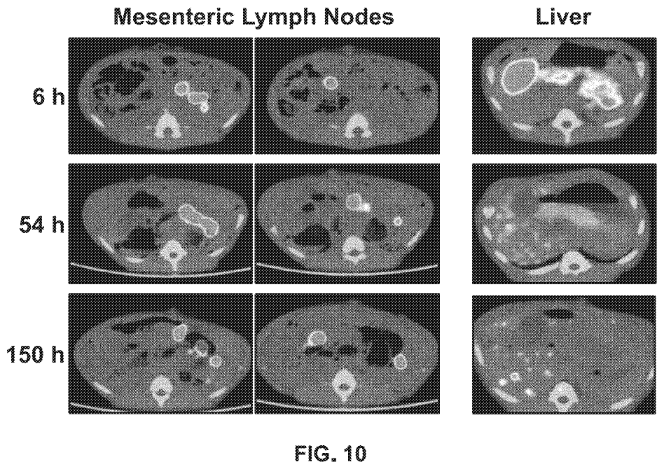

To demonstrate that hepatocytes are able to engraft in lymph nodes in a large animal model, a wild-type pig underwent a partial hepatectomy, and harvested hepatocytes were labeled with ⁸⁹Zr (half-life 78.4 h) prior to transplantation into 10-20 mesenteric lymph nodes. Radiolabeling efficiency was ˜20% and radioactivity concentration was ˜0.1 MBq/10⁶ cells. The animal received 6×10⁸ hepatocytes through direct mesenteric lymph node injection. PET-CT imaging at 6 h post-transplantation demonstrated presence of radioactivity within mesenteric lymph nodes (261.8±108.7 SUV; FIG. 10). Radioactivity remained present within the mesenteric lymph nodes at the 54 and 150 h time points (101.1±34.7 and 70.0±26.4 SUV, respectively). Background activity was measured in the left lumbar paraspinal muscle (0.1 SUV). Interestingly, although no radioactivity was detected within the liver at the 6 h time point, increasing amounts of radiotracer presence was found in the liver at both the 54 and 150 h time points (3.3±0.4 and 5.9±0.8 SUV, respectively), suggesting possible migration of transplanted cells to the liver.

Ex Vivo Corrected Hepatocytes are Able to Cure a Pig Model of HTI after Ectopic Transplantation into Lymph Nodes

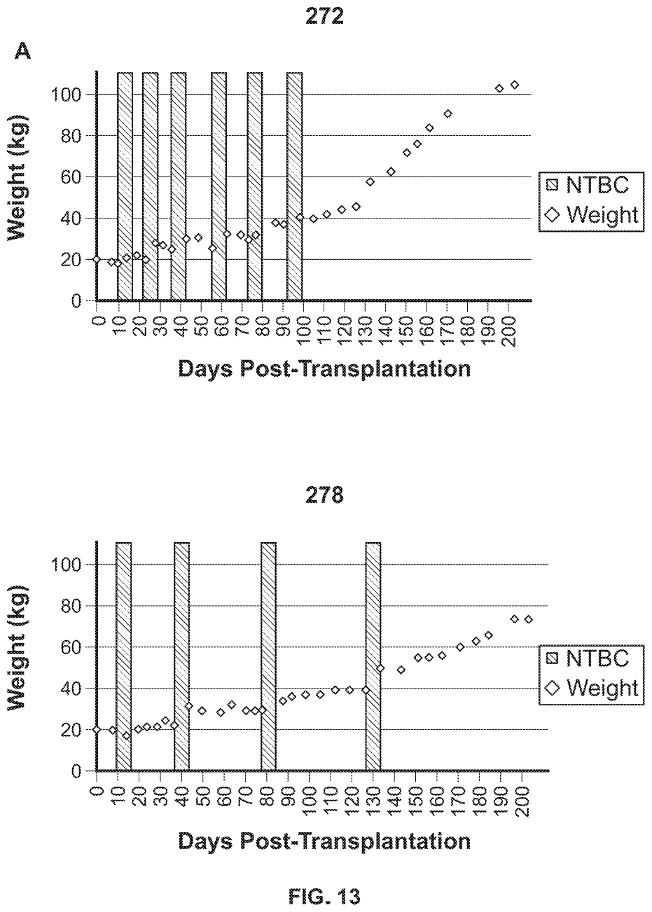

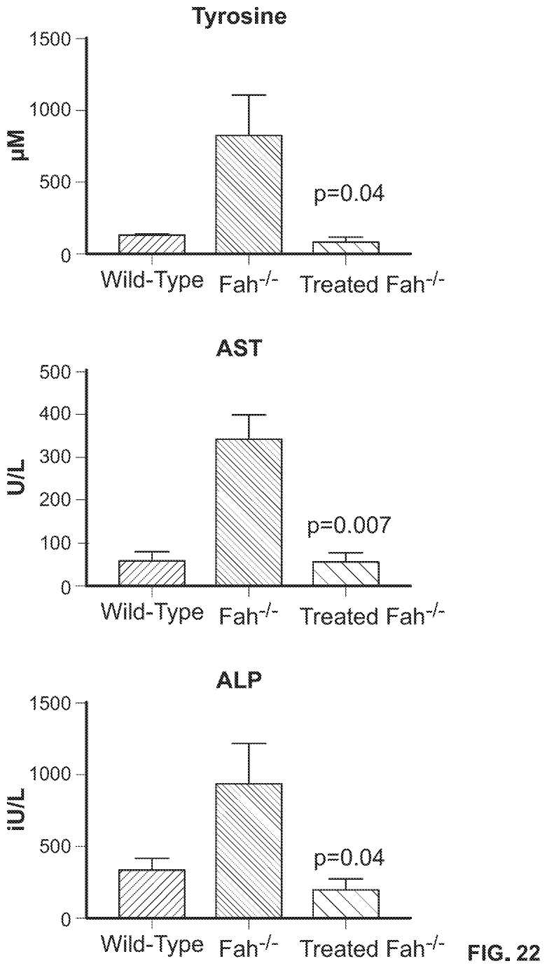

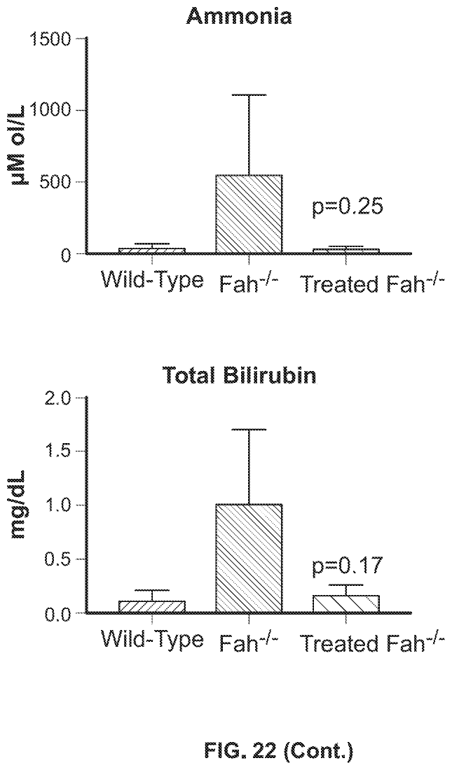

Animals remained on NTBC until the time of transplantation, at which point administration of NTBC was discontinued to stimulate expansion of the newly transplanted FAH-positive hepatocytes. Animals were then cycled on and off NTBC based on weight parameters until an NTBC-independent growth was achieved (FIG. 13). NTBC-independent growth was attained at a mean of 135±25 days post-transplantation (range: 97 to 161 days), after 3 to 6 cycles of NTBC. Biochemical cure of the HT-1 phenotype was confirmed by measurement of liver specific enzymes known to be elevated in HT-1 as well as normalization of tyrosine levels. At the time of euthanization, tyrosine (84.2±34.5 M) levels in our five treated pigs were within normal limits for a wild-type animal, and were significantly lower than untreated FAH^{-/-} controls (FIG. 22). Similar results were seen in liver function tests in treated animals compared to untreated controls AST (56.8±21.7 U/L), ALP (186.6±78.8 IU/L), ammonia (35.8±14.4 μmol/L), albumin (3.6±0.3 g/dL), and total bilirubin (0.15±0.1 mg/dL) levels.

Hepatocytes Transplanted into Lymph Nodes Demonstrate Long-Term Survival

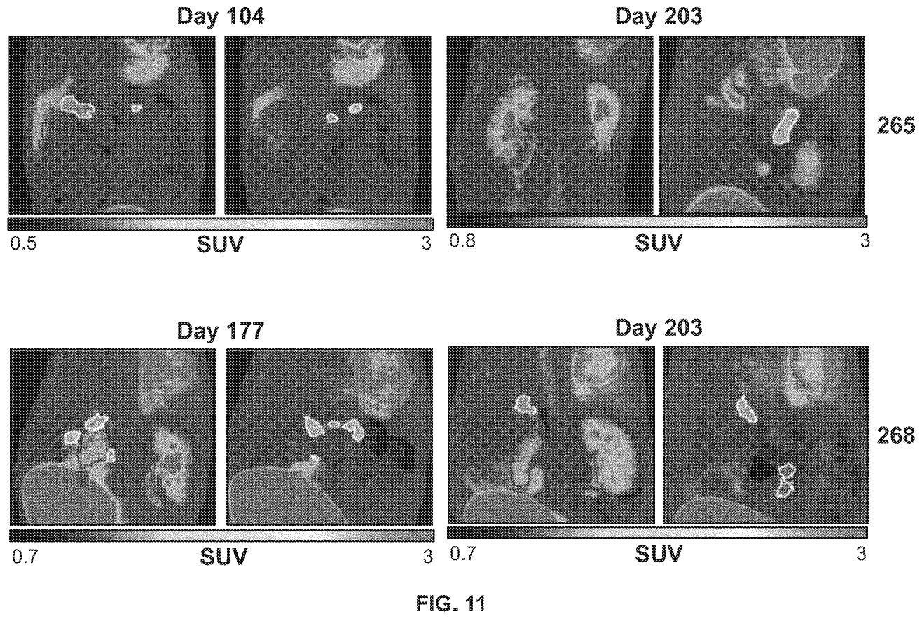

To confirm that transplanted hepatocytes were still present in mesenteric lymph nodes at later time points, two of our animals were co-transfected with a lentiviral vector carrying the Fah transgene and a second lentiviral vector carrying the NIS reporter gene. We performed longitudinal PET-CT imaging of these two treated pigs to monitor for the expansion of NIS-positive hepatocytes in the mesentery and other tissues. One hour prior to imaging, animals received 10 mCi of Technetium99-m. Pigs 265 and 268 were scanned at 104 and 203 days and 177 and 203 days respectively post-transplantation. Both animals showed NIS-positivity in mesenteric lymph nodes (15.7 and 39.0 SUV and 92.6 and 32.0 SUV, respectively; FIG. 11). No NIS-positive signal was found in the liver at any time point.

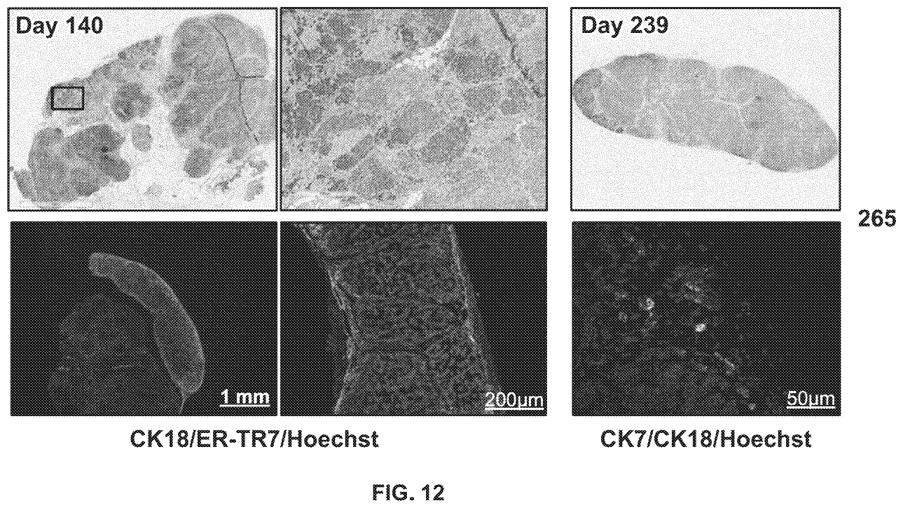

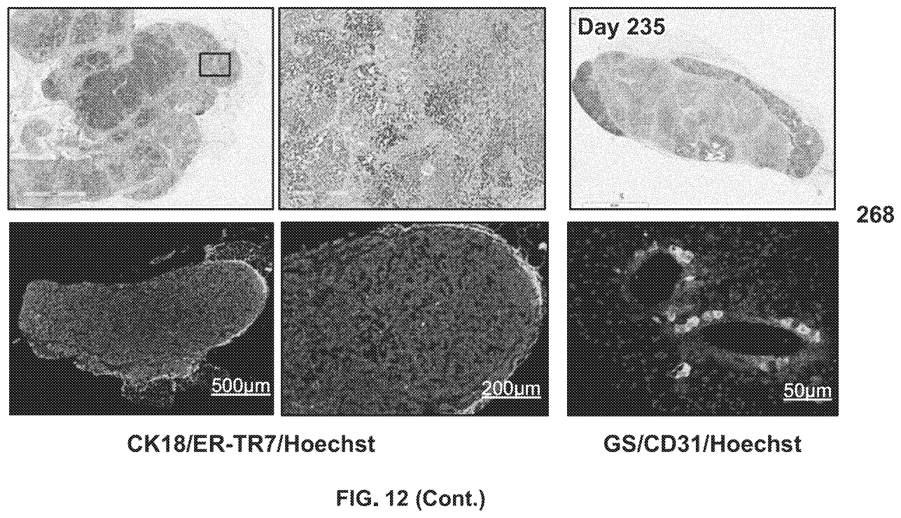

To correlate these data with histological findings, lymph node biopsies were performed on two animals (265, 268) at day 140 post-transplantation. All lymph nodes at the root of the mesentery were grossly positive for hepatocyte tissue. IHC for FAH-positive hepatocytes was in all samples taken within the mesenteric lymph nodes in both animals (FIG. 12). Transplanted hepatocytes were more commonly found in a subcapsular location within the lymph node. All experimental animals were euthanized at 212 to 241 days post-transplantation. Histology from mesenteric lymph nodes showed sustained presence of FAH-positive hepatocytes in four out of five animals (FIG. 12), including the two animals that had been biopsied previously. Hepatocyte presence was confirmed by IHC for FAH and cytokeratin 18. CD31+ endothelial cells were found within areas of hepatocyte engraftment, suggesting that significant blood vessel remodeling took place after transplantation to sustain long-term engraftment. Positivity for glutamine synthetase, an enzyme exclusively expressed in pericentral hepatocytes in mammals, was found in hepatocytes surrounding CD31+ neo-vessels, suggesting neo-formation of a central vein. Reproduction of hepatic microarchitecture within transplanted lymph nodes is further supported by presence of cytokeratin 7-positive bile duct cells within the areas of engraftment.

Hepatocytes Transplanted into Lymph Nodes Migrate to the Liver

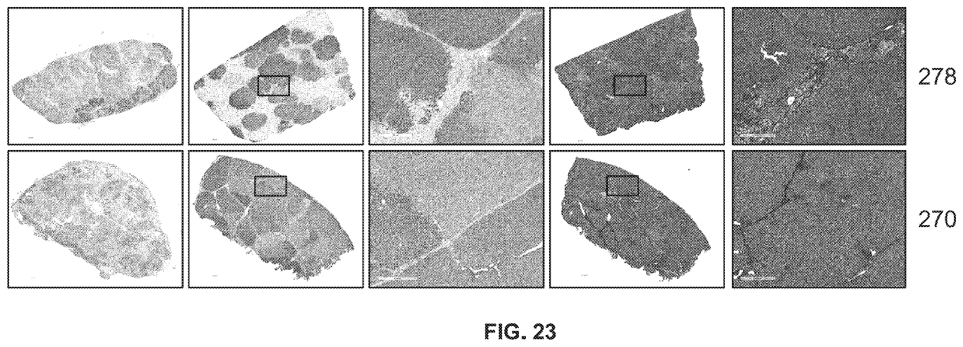

Migration of hepatocytes to the liver, suggested by the ⁸⁹Zr 54 and 150 h scanning, was confirmed by FAH immunohistochemistry of liver tissue demonstrating multiple FAH-positive nodules within all five livers, covering 67 to 100% (84.7±7.0) of the total liver area. A negative correlation was found between percent liver repopulation with FAH-positive hepatocytes and sustained presence of FAH-positive areas of hepatocyte engraftment within lymph nodes. Pigs 270 and 272 with complete liver repopulation with FAH-positive hepatocytes showed little to no hepatocyte presence remaining within lymph nodes at the terminal time point, while pigs with only partial liver repopulation with FAH-positive hepatocytes demonstrated a more robust hepatocyte presence within the lymph nodes at 235-239 days post-transplantation. As expected, FAH-negative areas of the liver showed marked hepatocellular damage and fibrosis due to prolonged NTBC withdrawal. However, the two fully FAH-positive livers demonstrated healthy, normal-looking tissue with minimal residual fibrosis, suggesting that the hepatic insult that occurs during NTBC cycling is reversible with time, as FAH-positive hepatocytes expand to repopulate the liver (FIG. 23).

In order to characterize any differences between the hepatocytes that migrated to the liver and those that remained in the lymph nodes, we performed next generation sequencing and bioinformatics analysis of both cell groups. Mapping statistics are provided in Table 2.

TABLE 2
Next generation sequencing mapping statistics

| | | | Integration | Integration |
|---|---|---|---|---|
| | | Mapped Reads | Points | Points |
| Sample | Total Reads | (Total) | at 1X | at 5X |
| Lymph | 115.748.080 | 5,465,009 (7.34%) | 40.239 | 6.335 |
| node | | | | |
| Liver | 116.490.845 | 21,126,444 (22.29 %) | 27.987 | 4.270 |

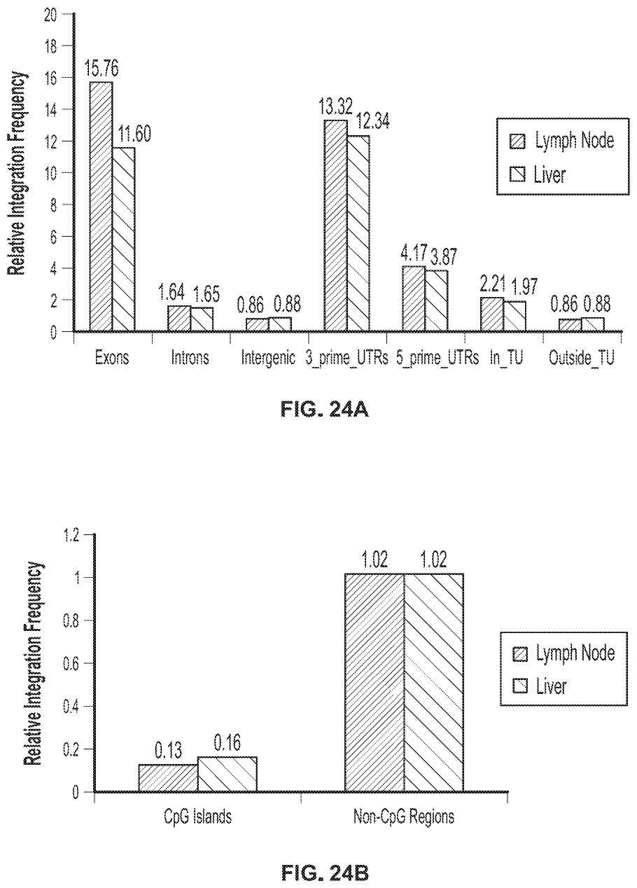

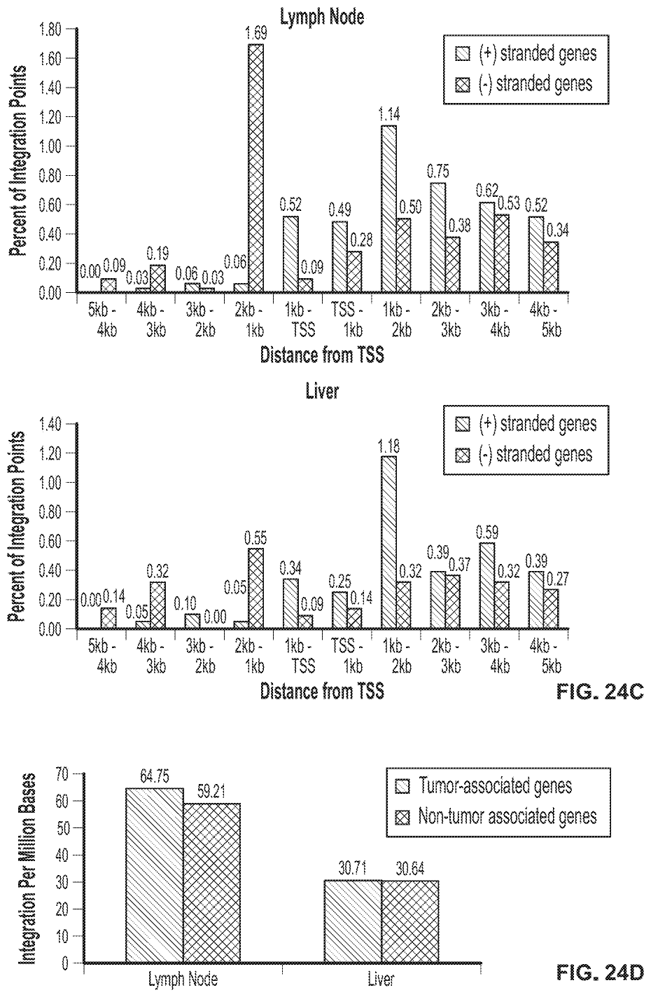

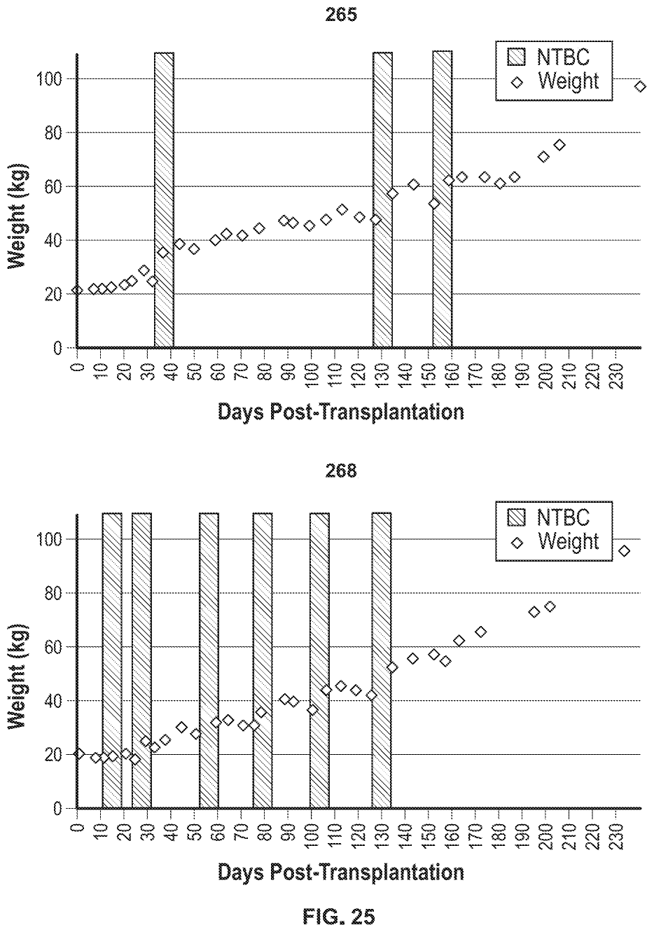

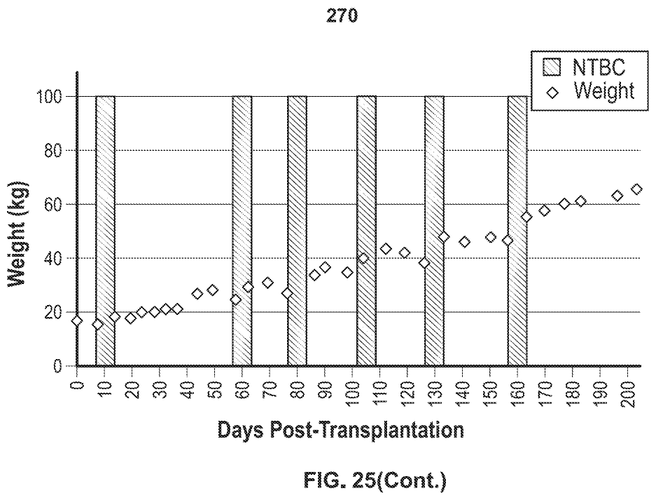

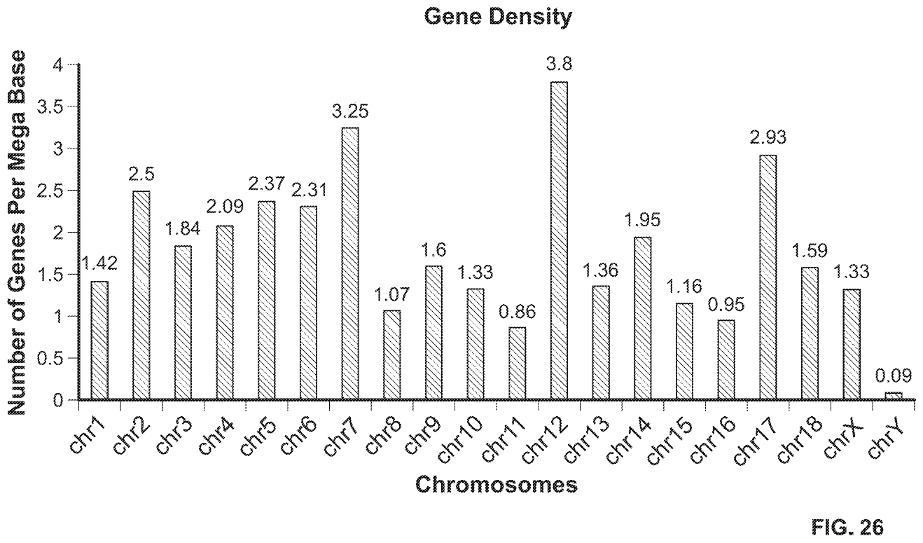

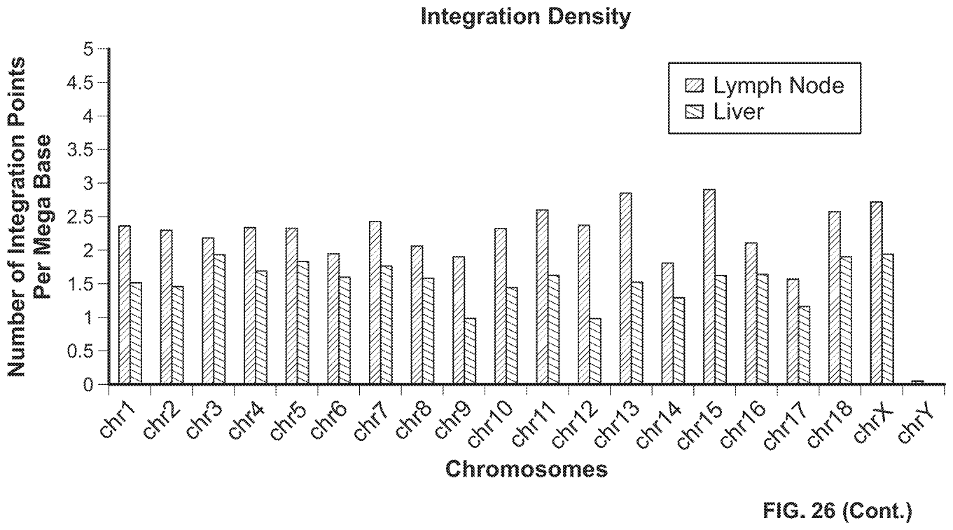

We found no significant differences in lentiviral integration profile between these two cell populations. In both cases, integration occurred more often in coding regions than in non-coding regions of the genome. Integration also occurred more often in exons than in introns. (FIG. 24A). Therefore, integration was favored in chromosomes with higher gene densities. In both cell populations, lentiviral integration was clearly favored downstream of transcription start sites, and was rare in CpG-rich islands (FIG. 24B). There was no preference for integration in tumor-associated genes in either cell population (FIG. 26). The genes with the highest integration frequency in both groups of cells are presented in Table 3.

TABLE 3
Genes with most frequent LV-FAH integration

| Lymph node hepatocytes | | | | | Liver hepatocytes | | | | |
|---|---|---|---|---|---|---|---|---|---|
| | Number of | Percent of | | | | Number of | Percent of | | |
| | integration | integration | Number | Percent | | integration | integration | Number of | Percent of |
| Top genes | sites | sites | of reads | of reads | Top genes | sites | sites | reads | reads |
| GLRX2 | 26 | 0.40 | 338,309 | 25.57 | MIR9799 | 18 | 0.41 | 5,479,826 | 56.29 |
| MIR9799 | 14 | 0.21 | 148,852 | 11.24 | NDUFV2 | 53 | 1.20 | 1,669,810 | 17.15 |
| LNPK | 53 | 0.81 | 146,239 | 11.05 | IFRD1 | 45 | 1.02 | 952,259 | 9.79 |
| PDCD10 | 81 | 1.24 | 129,982 | 9.82 | SUCLG1 | 24 | 0.54 | 707,921 | 7.27 |
| BEX1 | 51 | 0.78 | 41,620 | 3.15 | CENPP | 77 | 1.75 | 328,807 | 3.38 |
| NDUFV2 | 25 | 0.38 | 39,429 | 2.98 | CPX2 | 36 | 0.82 | 78,896 | 0.81 |
| IDO1 | 5 | 0.08 | 30,360 | 2.29 | CTDSP2 | 45 | 1.02 | 41,364 | 0.42 |
| FAH | 206 | 3.15 | 28,545 | 2.16 | RAB28 | 19 | 0.43 | 23,253 | 0.24 |
| IFRD1 | 31 | 0.47 | 22,270 | 1.68 | QKI | 7 | 0.16 | 16,777 | 0.17 |
| BMP5 | 35 | 0.54 | 19,409 | 1.47 | GSTO1 | 21 | 0.48 | 14,074 | 0.14 |
| MTRF1 | 8 | 0.99 | 19,077 | 1.44 | ENY2 | 30 | 0.68 | 12,470 | 0.13 |
| SUCLG1 | 53 | 0.15 | 19,033 | 1.44 | PRMT6 | 26 | 0.59 | 12,390 | 0.13 |
| ADAMTS1 | 5 | 0.26 | 17,754 | 1.34 | C1QTNF3 | 49 | 1.11 | 11,544 | 0.12 |
| HSD17B3 | 32 | 0.54 | 16,484 | 1.25 | RGS5 | 13 | 0.30 | 11,377 | 0.12 |
| SLC9A2 | 15 | 0.12 | 14,970 | 1.13 | MTRF1 | 36 | 0.82 | 10,972 | 0.11 |
| ACVR2A | 11 | 0.81 | 13,296 | 1.00 | SNCAIP | 5 | 0.11 | 10,252 | 0.11 |
| KCNJ2 | 21 | 0.08 | 12,294 | 0.93 | CTSV | 5 | 0.11 | 9,865 | 0.10 |
| CLDN22 | 8 | 0.48 | 12,073 | 0.91 | FAH | 135 | 3.06 | 9,650 | 0.10 |

To determine whether the transcriptome of engrafted lymph nodes is related to expression within normal liver tissue, hepatized and control lymph nodes from experimental animals were compared with native liver from experimental animals and control liver from control animals. Total RNA was extracted and RNAseq was performed with three independent biological replicates. An initial analysis to identify the genes differentially expressed (DE) between tissues was performed using Partek Genomic Suite. According to the employed cut-off (log 2|FC|≥>=2, FDR>=0.05), 12000 genes were identified as DE and contrasting control lymph node to liver tissues including hepatized lymph nodes. T-distributed Stochastic Neighbor Embedding (t-SNE) showed a strong tendency of hepatized lymph nodes transcripts to cluster with liver tissues samples, whereas control lymph nodes were distinct from the other three groups. These data suggest that a significant proportion of the lymph node transcriptome changes to liver-like expression levels after hepatocyte transplantation.

### Discussion

In our study, we show that hepatocyte transplantation into lymph nodes is able to generate enough liver mass to correct the HT1 defect, suggesting that hepatocyte transplantation into lymph nodes is scalable to large animals and may therefore be a clinically relevant technique that permits the creation of sufficient ectopic liver mass to significantly impact liver function and even cure metabolic disease.

We used autologous hepatocytes obtained from a partial hepatectomy and corrected through ex vivo gene therapy. This technique could be translated into human patients with genetic liver disease, thereby avoiding immune concerns related to the use of allogeneic cells. At the same time, in our study hepatocytes were transplanted into pig mesenteric lymph nodes using a minimal open technique due to anatomical limitations of the model, but the same procedure could be performed into central or peripheral lymph nodes in humans via a percutaneous, ultrasound-guided technique. Outside of ex vivo gene therapy for the treatment of metabolic liver disease, allogeneic hepatocyte transplantation into lymph nodes could generate enough ectopic hepatic mass to stabilize liver function as a bridge to either whole organ liver transplantation or, in some cases, regeneration and recovery of the native liver. Regarding the HT-1 pig model, these animals show evidence of acute and chronic liver failure early in the disease, thereby replicating many of the features seen in other liver diseases. The fact that our animals were rescued and recovered completely after the transplantation procedure validates the robustness of this approach for human diseases. The animals treated through this method demonstrated NTBC independence within three to five months of treatment, as well as complete normalization of tyrosine levels and liver function tests.

If hepatocyte transplantation were to be performed in animals with fibrotic or cirrhotic livers, it is possible that a cure of the metabolic disease could occur faster with hepatocyte delivery ectopically into lymph nodes than orthotopically into the native, diseased liver. Furthermore, the current data show that ectopic transplantation is supportive of native liver repopulation over time.

We found that although hepatocytes demonstrated long-term survival within lymph nodes, some also traveled to and engrafted in the liver as early as two days post-transplantation. This phenomenon was not seen in previous mouse studies and could be related to the microanatomy and direction of lymph flow in pig lymph nodes, which differ from those of other mammals: their microarchitecture is inverted, with the germinal centers being located internally to the medulla, suggesting that lymphocytes are transported back into a capillary system after passing through a lymph node, as opposed to into a lymphatic system as in other animals In our study, the fact that hepatocytes engrafted in both lymph node and liver makes it impossible to determine whether the expansion of transplanted hepatocytes to the levels necessary to correct metabolic disease occurred within the lymph nodes, within the liver, or in both sites. Central lymph node injection could potentially be considered a safer alternative to portal vein injection for hepatocyte delivery into the liver, since portal vein infusion of cells often leads to thrombotic complications and requires systemic anticoagulation.

Based on these results, we also analyzed in detail the integration profile of our lentiviral vector in both hepatocytes that remained in the lymph nodes and hepatocytes that migrated to the liver. No significant differences were seen between the two cell populations suggesting that selection of sub cell population after ex vivo gene therapy did not occur. In both cell groups, the lentiviral vector showed a benign integration profile, with no preference for integration in promoter regions or tumor-associated genes. Of note, the FAH gene was within the top twenty genes with the highest integration frequency in both groups, suggesting that homology may to some extent be driving integration. We also examined relative changes in gene expression, finding no significant differences in transcriptomes between engrafted and control lymph nodes, as well as engrafted and control liver, implying that ex vivo lentiviral gene therapy does not alter gene expression in corrected cells. Together, these two findings advocate for the safety of the ex vivo lentiviral gene therapy approach.

In summary, we have shown that transplantation of corrected, autologous hepatocytes into mesenteric lymph nodes after ex vivo gene therapy is curative in the pig model of HT1. This method is not only safe, with no complications related to the procedure in any of the animals treated, but is able to correct the metabolic disease in the same amount of time as when hepatocytes are transplanted back into the native liver via the portal vein. Hepatocyte transplantation into lymph nodes is a promising approach to the treatment of liver disease that holds several important advantages over more traditional cell transplantation techniques, especially in patients with pre-existing liver damage and fibrosis.

### Example 6

We have demonstrated curative ex vivo gene and cell therapy using a lentiviral vector to express FAH in autologous hepatocytes. To further evaluate the long-term clinical outcomes of this therapeutic approach, we continued to monitor one of these pigs over the course of three years.

### Materials & Methods:

### Animals and Animal Care:

All animals received humane care in compliance with the regulations of the institutional animal care and use committee at Mayo Clinic, Rochester, Minn. Daily observations were performed by animal care/laboratory staff and any clinical concerns were addressed by on-site veterinarians. Animals were weighed weekly until NTBC independence was confirmed and then at various time points throughout the study. Since growing pigs are prone to gastric ulceration and NTBC cycling can cause inappetance, animals were given prophylactic omeprazole orally at a dose of 1 mg/kg with a maximum dose of 80 mg/day for the duration of the study. At the onset of sexual maturity, semen collection was carried out weekly with average yield and concentration evaluated on a SpermaCue photometer (MiniTube of America, Delavan, Wis.).

### Biochemical Analysis:

For biochemical analysis of alkaline phosphatase (ALP), alanine aminotransferase (ALT), aspartate aminotransferase (AST), albumin, and total bilirubin (TBIL), plasma or serum was analyzed with the VetScan VS2 benchtop analyzer (Mammalian Liver Profile, Abaxis, Union City, Calif.) according to the manufacturer's instructions. Alpha-fetoprotein (AFP) was analyzed in serum with the Beckman Coulter Access AFP immunoenzymatic assay on the Beckman Coulter UniCel DXI 800 (Beckman Coulter Inc., Fullerton, Calif.). Tyrosine values were determined using tandem mass spectrometry and chromatography via Mayo Clinic's internal biochemical PKU test.

### Histology Analysis:

For histological analysis, tissue samples were fixed in 10% neutral buffered formalin (Protocol, Fisher-Scientific, Pittsburgh, Pa.) and processed for paraffin embedding and sectioning. For hematoxylin and eosin staining, slides were prepared with standard protocols. FAH or Ki67 immunohistochemistry using a polyclonal rabbit anti-FAH primary antibody (9) or a monoclonal anti-Ki67 primary antibody (MIB-1; Dako/Agilent, Santa Clara, Calif.) was performed with a Bond III automatic stainer (Leica, Buffalo Grove, Ill.) with a 20-min antigen retrieval step using Bond Epitope Retrieval Solution 2 (Leica, Buffalo Grove, Ill.), and stained with diaminobenzidine (Leica, Buffalo Grove, Ill.). Slides used to quantify fibrosis were stained with Masson's trichrome stain using standard protocols. Fifteen rectangular areas totaling 2.39×10⁷ μm² per slide were randomly selected and analyzed using an Aperio ImageScope algorithm that quantifies pixel hue. Blue pixels were counted as being positive for collagen. Reported results are total percentage of positive pixels among those analyzed. Similarly, Ki-67 quantification was performed by selecting 15 random rectangular areas totaling 2.39×10⁷ μm² per slide. Areas were analyzed and quantified using an Aperio ImageScope algorithm that quantifies nuclear staining. Results are reported as percentage of nuclear positivity among cells analyzed.

### Statistical Analysis:

Data were analyzed using GraphPad Prism software version 7. Experimental analyses with only one comparison were compared using a two-tailed Welch's t test. Differences between multiple groups were compared using a one-way ANOVA followed by Tukey's multiple comparisons test. P<0.05 was considered statistically significant.

### Results:

Pig Y842 remained phenotypically stable for the duration of the study. Y842 was a male homozygous Fah^{-/-} pig with a 50% Large White and 50% Landrace pedigree. At 39 days of age, weighing 11 kg, this pig underwent a partial hepatectomy to remove approximately 10% of its liver. Hepatocytes were isolated and transduced with a lentiviral vector (LV) expressing the porcine Fah cDNA under the control of a hepatocyte-specific promoter. LV-transduced autologous hepatocytes were transplanted back to the same pig by percutaneous portal vein injection. Y842 was cycled on/off the protective drug NTBC for four cycles, and became NTBC-independent 95 days after transplantation. At 12 months of age, liver biopsies and biochemical analyses revealed complete amelioration of symptoms characteristic of HT1.

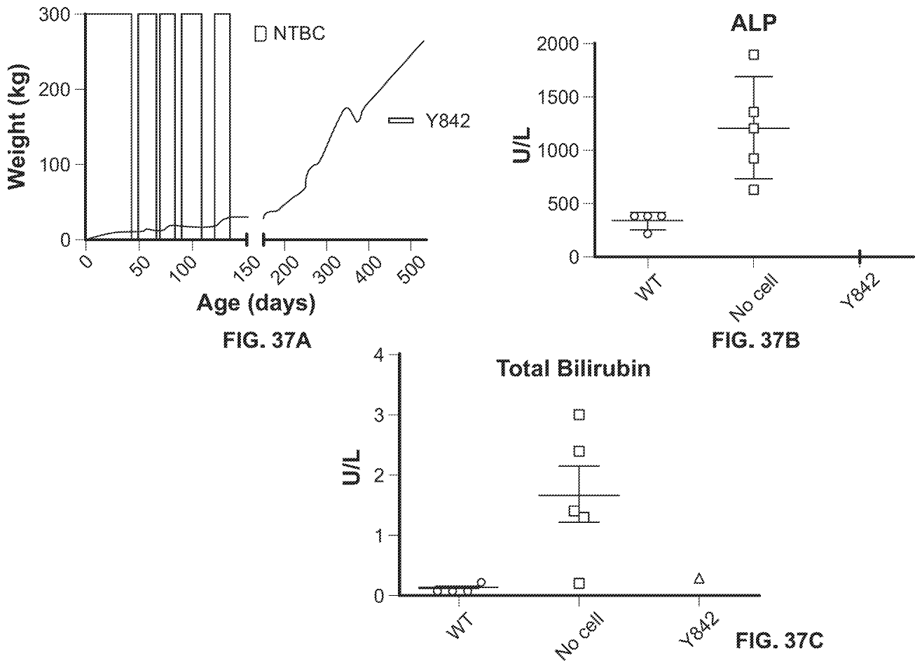

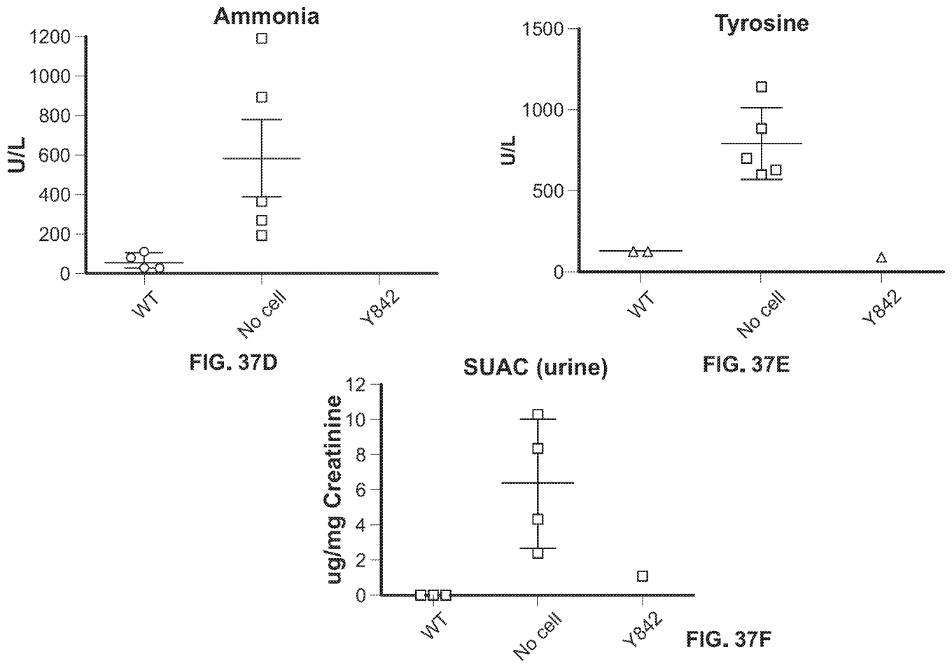

In this study, we continued to monitor the phenotype of Y842 over the course of 949 days after transplantation. At 532 days of age, the pig weighed 265 kg, which was at the functional limit of the available scale (FIG. 37A). The animal continued to thrive, and remained NTBC independent for the course of this study. As a measure of health, the animal maintained sexual fecundity, siring a total of 57 piglets from five breedings; the last litter of piglets was sired 860 days after transplantation. Semen collection was carried out weekly with an average yield of 275 ml at a concentration of 265×10⁶ sperm/ml. At 988 days after birth, Y842 was euthanized at the request of the institute's veterinarians owing to the inability to humanely house such a large animal. No clinical manifestations of liver disease were noted at the time of euthanasia. Biochemical parameters of liver function were normal.

At the time of euthanasia, a complete biochemical and histological analysis was performed. Due to the lack of appropriate age-matched controls (large pigs are not routinely kept at institutes for this prolonged period of time), we compared biochemical values from Y842 to younger historical Fah^{+/+} (wild type) and Fah^{-/-} (No Cell) controls (FIG. 37B-F). Biochemical liver function markers, including alkaline phosphatase (ALP), total bilirubin, and ammonia were within normal limits, indicating normal liver function. Next, we looked at metabolites specific to HT1. Tyrosine and succinylacetoacetate levels were also normalized indicating continued amelioration of tyrosine catabolism. Finally, to investigate whether any biochemical indication of HCC could be detected, we determined the concentration of serum AFP by ELISA to be 0.8 ng/ml, which was within normal limits.

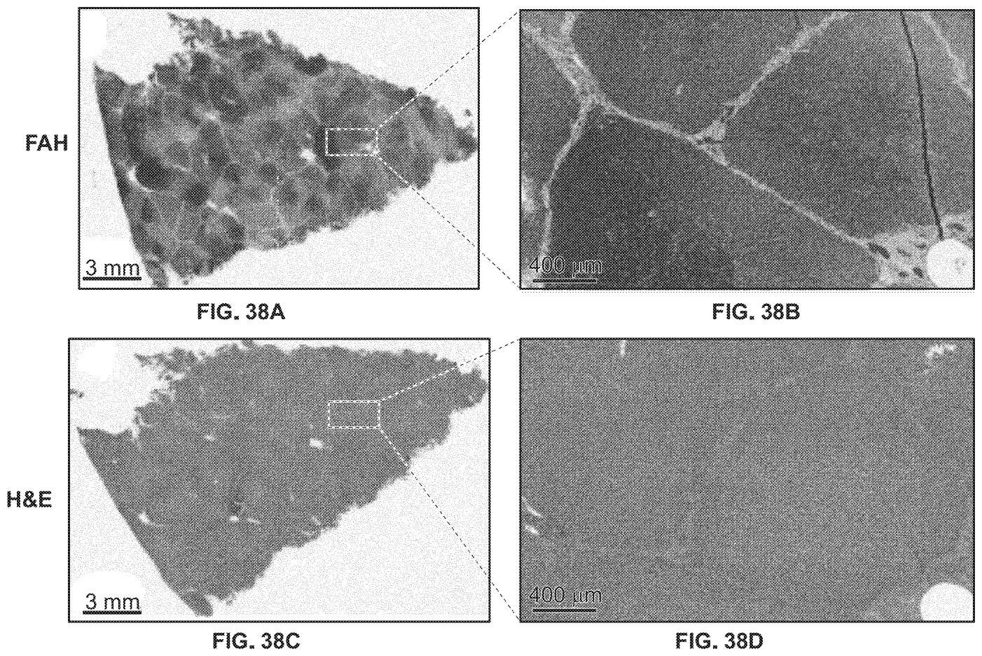

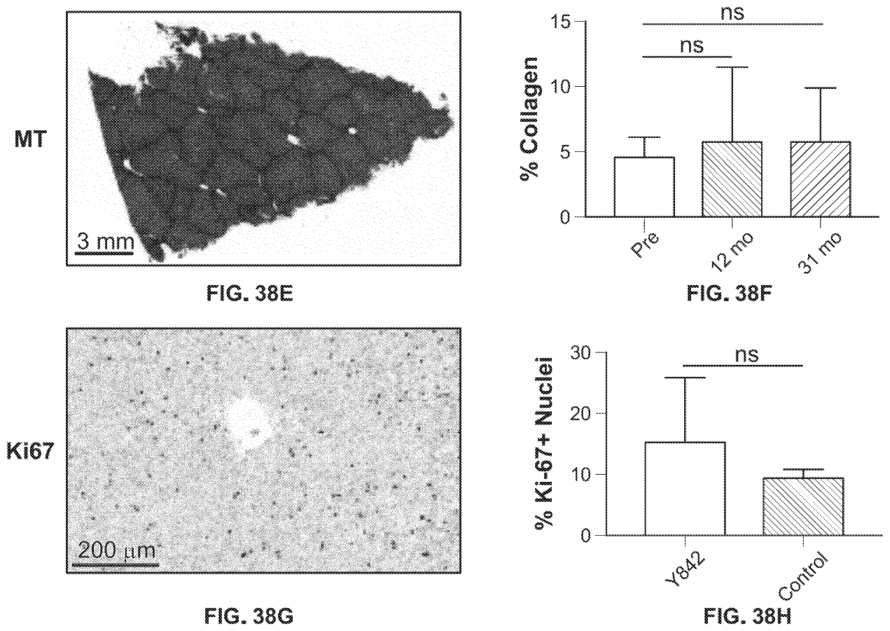

Histological assessment of the liver revealed normal tissue architecture. We next assessed by histology and immunohistochemistry whether any abnormal pathology could be detected in the liver. Macroscopically, the liver had no gross lesions or tumors and there was no evidence of surface nodularity or extrahepatic collateral vein development suggesting the absence of cirrhosis. FAH immunohistochemistry of the liver revealed complete repopulation of the Fah^{-/-} liver by the LV-Fah-transduced hepatocytes (FIG. 38, A-B). Dividing the liver into 1-2 cm sections and performing H&E (FIG. 38, C-D) and Masson's Trichrome staining (FIG. 38, E-F) throughout the liver revealed no significant pathology, including no evidence of HCC—supporting the negative AFP findings in the serum. Only minimal periportal fibrosis was noted, which was in contrast to the mild stage 1 fibrosis detected at the 12 month time point—providing provocative data that the mild fibrosis in HT1 may be reversible after FAH+ hepatocyte repopulation. Quantification of collagen staining revealed no significant changes in fibrosis over the course of 31 months, a striking contrast to Fah^{-/-} pigs that succumb to severe fibrosis within six months in the absence of intervention. Finally, we quantified Ki67-positive cells in the liver to determine if abnormal proliferation was occurring. No significant difference in Ki67-positive cells was noted between multiples segments of the liver from Y842 and a 42-month old control boar (FIG. 38, G-H).

Histological assessment of the kidney revealed normal tissue architecture. As histopathological abnormities of the kidney are commonly associated with HT1 clinically, we went on to analyze the kidneys of Y842 (data not shown). As expected, no FAH+ renal tubular cells were detected by IHC, and H&E staining revealed no significant pathologies except for a mild increase in tubular degeneration with regeneration. Additionally, primarily medullary, inner and outer stripe and some medullary rays were noted with an increase in medullary interstitial stroma. In addition to the liver and kidneys, no significant pathology was detected in the rest of the animal that would indicate an adverse event resultant from the ex vivo gene therapy (data not shown).

### Conclusions

This study demonstrated robust engraftment and proliferation of corrected cells in the Fah^{-/-} liver which completely cured the disease by preventing the onset of severe fibrosis and liver failure, which is characteristic of untreated Fah^{-/-} pigs. With this chronic follow up presented herein, we demonstrate that this phenotypic correction was durable and, in a single pig, did not result in cirrhosis or HCC, which is an important observation when considering the clinical potential of gene therapy for HT1. Indeed, the data described in this paper demonstrate conclusively that ex vivo gene therapy for HT1 is feasible, efficacious, and safe, providing a durable cure in a clinically relevant model of this disease.

### Example 7

### Introduction

In order to investigate the potential for adverse events such as insertional activation of oncogenes or the disruption of gene coding sequences, and to evaluate the genotoxicity of lentivirus mediated gene delivery into human hepatocytes, here we present a genome-wide analysis of the distribution of integration sites of a lentiviral vector carrying the human FAH gene (LV-FAH) in human hepatocytes.

### Materials and Methods

### Lentiviral Vector Design and Production

The lentiviral vector containing human FAH coding region and liver specific promoter (HCR-hAAT) was constructed. A 252 bp of SerpinAl Exon-1 was located in downstream of the promoter (FIG. 1). This lentiviral vector was then functionally confirmed by gene correction experiments in mice in vivo. The pLV-hFAH, pMD.G and p8.91 plasmids were used for cotranfection of 193T cell to produce lentiviral particles, using 1 mg/ml Polyethyleneimine (PEI, Polysciences) at 4:1 v/w ratio of PEI: DNA. The lentiviral supernatants were harvested from cell culture medium (DMEM with 10% FBS, 25 mM HEPES) at 48 and 72 h after transfection, mixed together, filtered through 0.45 um filters, concentrated by 50% Polyethylene glycol (PEG)-6000 (Sigma) precipitation and centrifugation, and immediately aliquoted, stored frozen at −80° C. Lentivirus solution was titrated on 293T cells. Viral titers were in the level of 108 IU/ml.

### Human Hepatocytes and Lentiviral Vector Transduction

Fresh human hepatocytes were obtained from Yecuris, purity >95%, viability >70%. Hepatocytes were washed twice with cold culture medium, HBM (Lonza; # CC-3199) supplemented with HCM SingleQuotes (# CC-4182) and 5 mM Dexamethasone (Sigma). Hepatocytes were transduced with lentiviral vector at multiplicity of infection (MOI) 60, at 4° C., 10 rpm rotation for 90 minutes, then at 37° C. for 30 minutes. Transduced hepatocytes were plated and cultured on pre-coated (Coating Matrix Kit, Gibco) 6-well Primaria dishes at 1×10⁶ cells per well. Change medium 2 h after seeding and then every day.

### Isolation of Integration Sites and Next Generation Sequencing

Lentivirus transduced hepatocytes were harvested at 72 h after transduction. Gentra Puregene Cell Kit (QIAGEN) was used to lysate the cells and extract genomic DNA. To determine vector integration sites on the human genome, DNA fragments from host-vector junction were prepared using ligation-mediated PCR [21]. Briefly, five microgram each genomic DNA was digested with MseI (NEB). After purification, the digested DNA fragments were ligated with linkers. To avoid PCR amplification of the internal vector fragment downstream of the 5′ LTR, linker added DNA sample was treated with ApoI (NEB). Sample was then amplified by nested PCR. PCR primers and linker sequences are shown in Table 4 and FIG. 1.

TABLE 4

MseI linker:
Sense:
5′Phos-TAGTCCCTTAAGCGGAG-3′AmMO
Antisense:
5′-GTAATACGACTCACTATAGGGCCTCCGCTTAAGGGAC-3′
Linker primer (reverse primer):
5′-CAAGCAGAAGACGGCATACGAGATCGGTCTCGGCATTCC
TGCTGAACCGCTCTTCCGATCTGTAATACGACTCACTATAGG
GC-3′
Lenti-specific primer (LSP):
5′-TGTGACTCTGGTAACTAGAGATCCCTC-3′
Nested LSP:
5′-AATGATACGGCGACCACCGAGATCTACACTCTTTCCCTA
CACGACGCTCTTCCGATCTCGATGTGAGATCCCTCAGACCCT
TTTAGTCAG-3

PCR amplicons were gel separated, and 300 bp to 800 bp DNA fragments were purified using Qiagen gel extraction kit. The pooled PCR product was subjected to next generation sequencing by Illumina HiSeq2500. Utilize a spike-in of 30% PhiX DNA, paired-end 150 bp sequencing.

### Results

### Distribution of 79, 718 Unique Lentiviral Vector Integration Sites on the Human Hepatocyte Genome

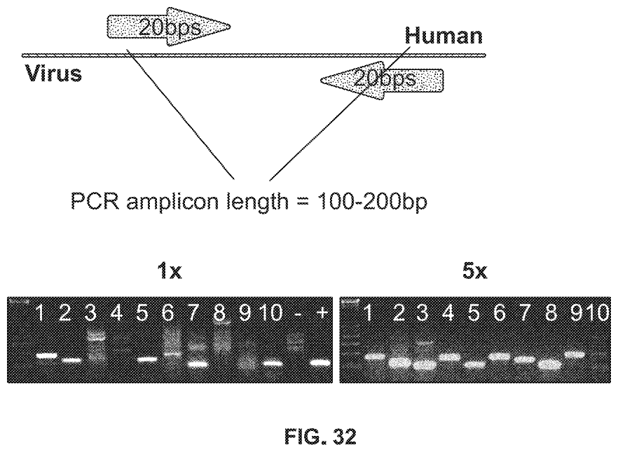

In our present study, 78.7 million lentivirus-host DNA junction sequences were read by next generation sequencing and mapped to human genome. Among them, 190,124 unique integration sites were identified at 1× read trimming level; 79,718 unique integration sites were identified at 5× reads trimming level. (FIG. 32) To avoid sequencing errors, these 79,718 unique integration sites were further characterized and reported in this study.

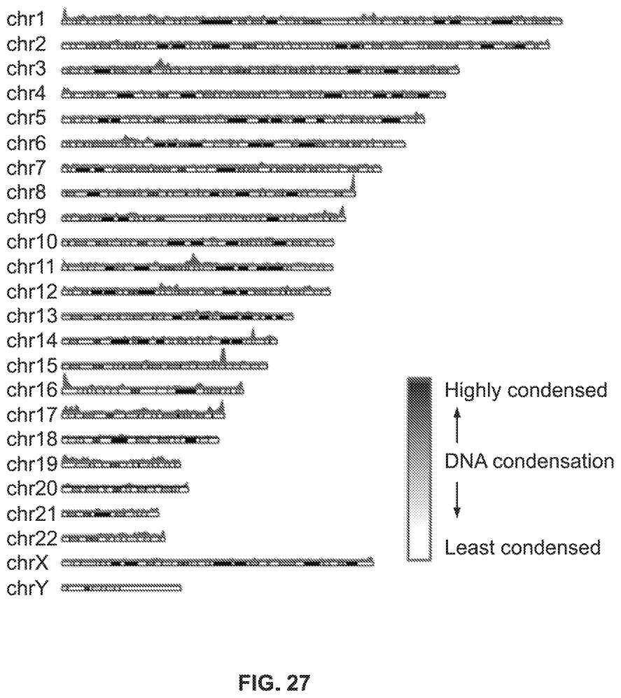

Patterns of provirus distribution in each chromosome were shown in FIG. 27. Lentivirus widely integrated in all chromosomes (except the Y). No gross significant integration preferences were observed in each chromosome even if some regional hotspots were detected. Interestingly, the regional hotspots in chromosome 15 and 14 correspond to the locations of FAH gene and SerpinAl gene, respectively. When comparing highly condensed and less condensed regions in each chromosome, most hotspot regions correspond to lighter band regions of the chromosomes indicating the presence of integration sites in less condensed regions of the genome (more accessible and more active). A tendency to a differential distribution of provirus towards telomeric and subtelomeric regions of the most of chromosomes was observed. Due to the uncertainty of human centromeric regions, no integration sites were mapped in those regions even though other authors showed that centromeric alphoid repeat regions are disfavored as integration sites.

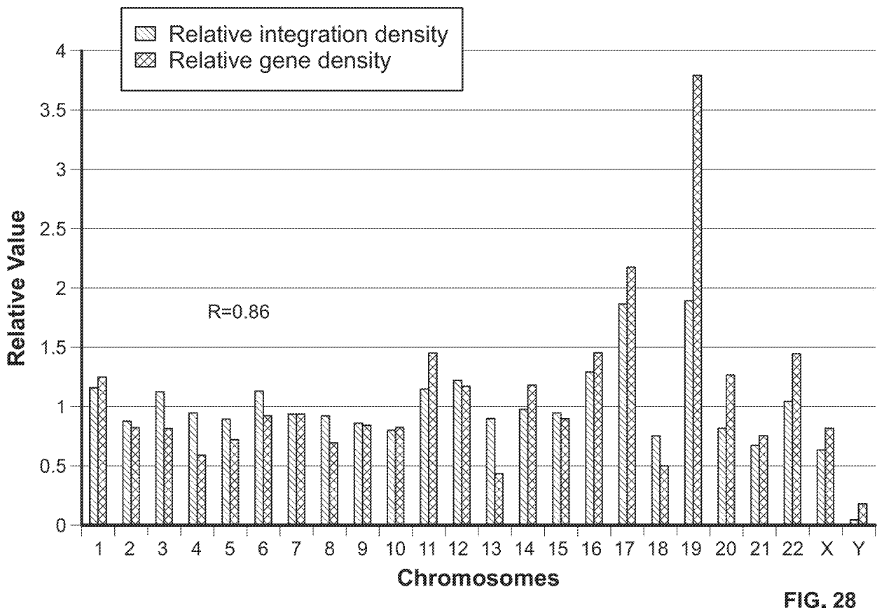

In order to know whether integration events are favored by gene-rich regions in chromosomes, a comparison between relative integration density and relative gene density was performed (FIG. 28). The correlation coefficient (R) of those two variables is 0.86, indicating that a high gene density in chromatin regions determine a favorable environment for integration.

### Lentiviral Integration Favored in Transcription Units, Disfavored in CpG Islands and Transcription Start Sites

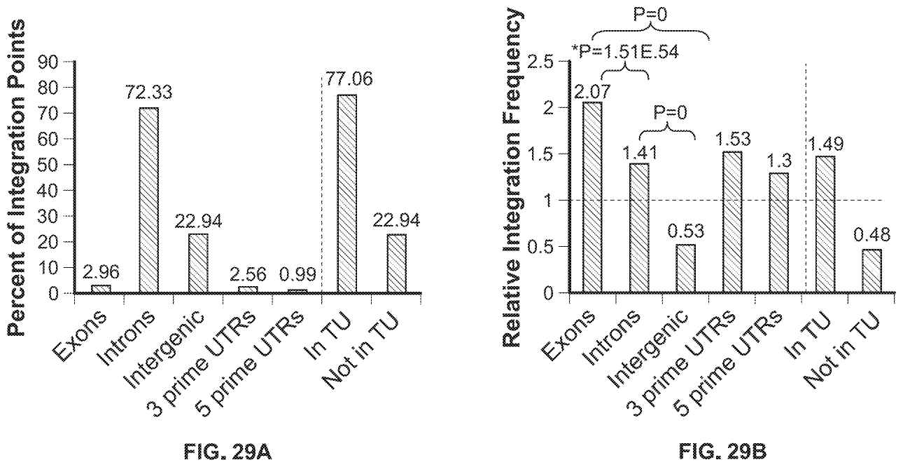

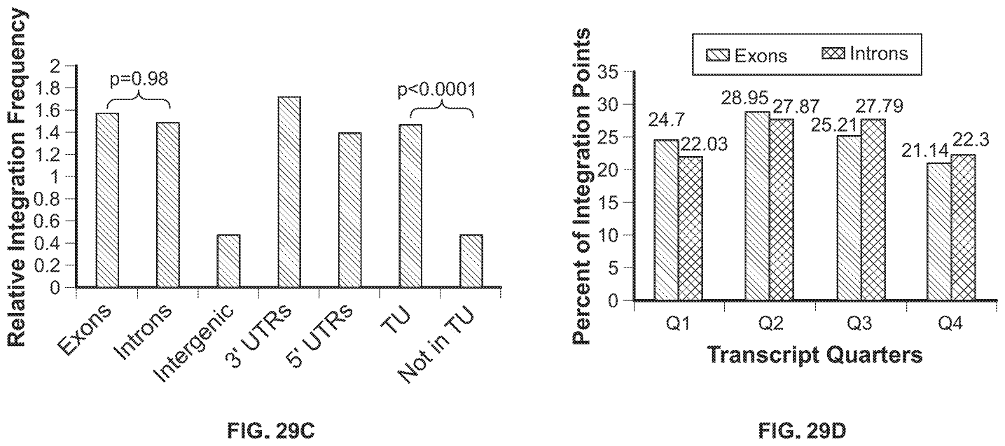

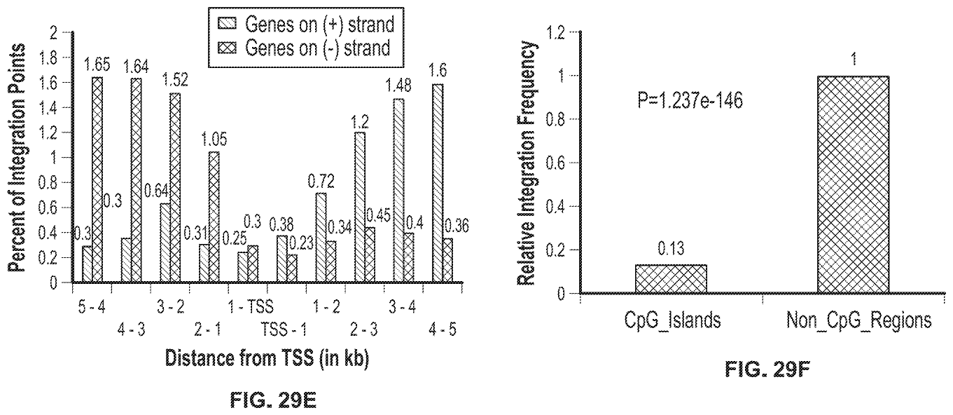

Analysis of the placement of the integration sites revealed that 77.1% located in transcription units (TU) (FIG. 29A), significantly different from intergenic regions (p<0.00xx). Within genes, integration was favored in introns over exons by percentage. Considering the greater size of intergenic and introns compared to TU and exons, we normalized all integration sites to the sizes of the features. Relative integration frequency above 1 indicates the enrichment of integration (FIG. 29B. Intergenic regions were disfavored as lentivirus integration (p=0 when compared with exons or introns). All elements in transcription units (exons, introns, 3′-UTRs and 5′-UTRs) were enriched, especially in the exons (p=1.5E-54 when compared with introns). This is likely a result of the contributions of the integration sites that happened in exons of FAH and SerpinAl genes. When we exclude the integration sites in these two genes (FIG. 29C), the difference of integration frequency between exons and introns was not statistically significant (p=0.96).

5′-UTR and 3′-UTR are important elements for the regulation of posttranscriptional activities, we analyzed them separately from exons. Integration events in 3′- and 5′-UTR were enriched significantly, especially 3′-UTR. We divided transcripts into four quarters (count exons and introns separately), the average percentages of integration sites across in features were shown in FIG. 29D. An equivalent distribution was seen across the transcripts in both exons and introns. No particular bias was found around 3′ and 5′ ends. Another important regulator for gene expression is transcription start site (TSS). We analyzed lentivirus integration in 5 kb upstream and downstream of TSS regions (FIG. 29E). No matter the genes are in sense or antisense strands, integration of lentivirus was disfavored at TSS and 5 kb upstream regions. Integration sites were enriched in 2 kb downstream and after, entering into transcription units. CpG islands are probable regulatory regions associated with gene 5′ ends, gamma-retrovirus reportedly showed a strong integration preference in or near these features. Integration by lentiviral vector was significantly disfavored within CpG islands (p=1.24E-146 when compared with non-CpG regions) (FIG. 29F).

### Actively Transcribed Genes are Preference for Lentivirus Integration

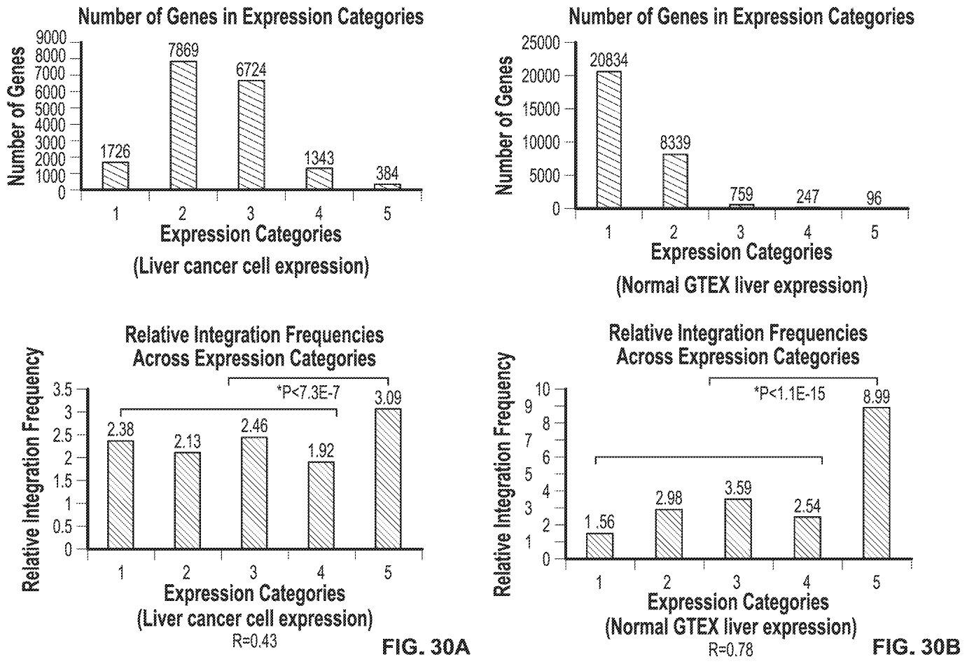

In this study, all genes were sorted into 5 categories based on their expression levels derived from publicly available RPKM data values. Two expression datasets were used to analyze the relationship between expression level and integration frequency, (A) HepG2 hepatocellular carcinoma from ATCC and (B) normal liver tissues from the genotype-tissue expression project (GTEx). In human hepatocytes transduced with therapeutic lentiviral vector carrying human FAH gene, we observed a significant preference of lentivirus to integrate in highly expressed genes (category 5, p<7.3E-7) in both expression datasets (FIGS. 30A and 30B), especially in normal liver tissue. The correlation coefficient between gene expression levels in normal liver tissue and relative integration frequency is 0.78.

### Tumorigenesis Genes are not Favored Targets for Lentiviral Vectors

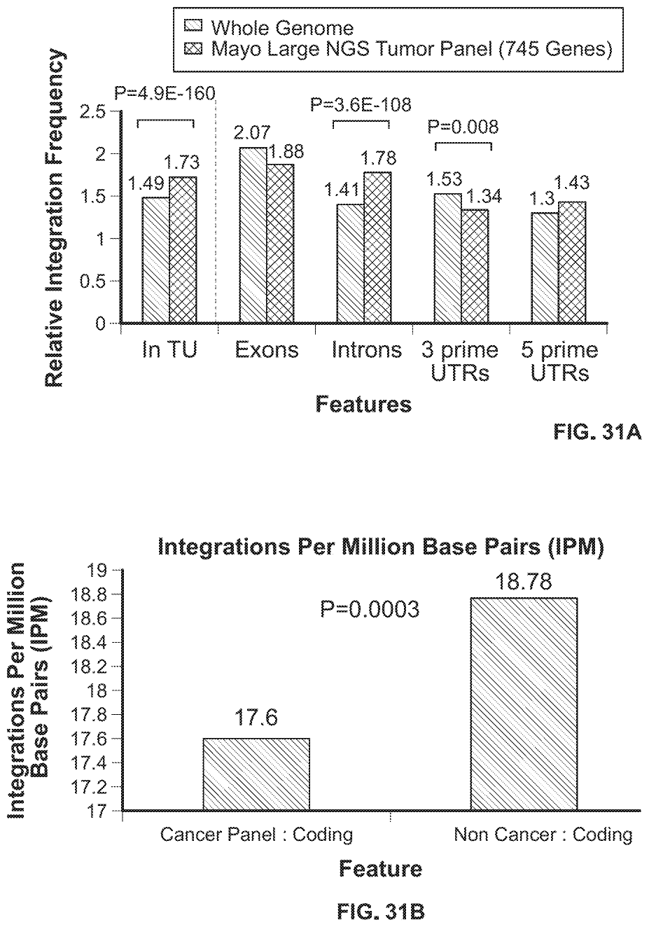

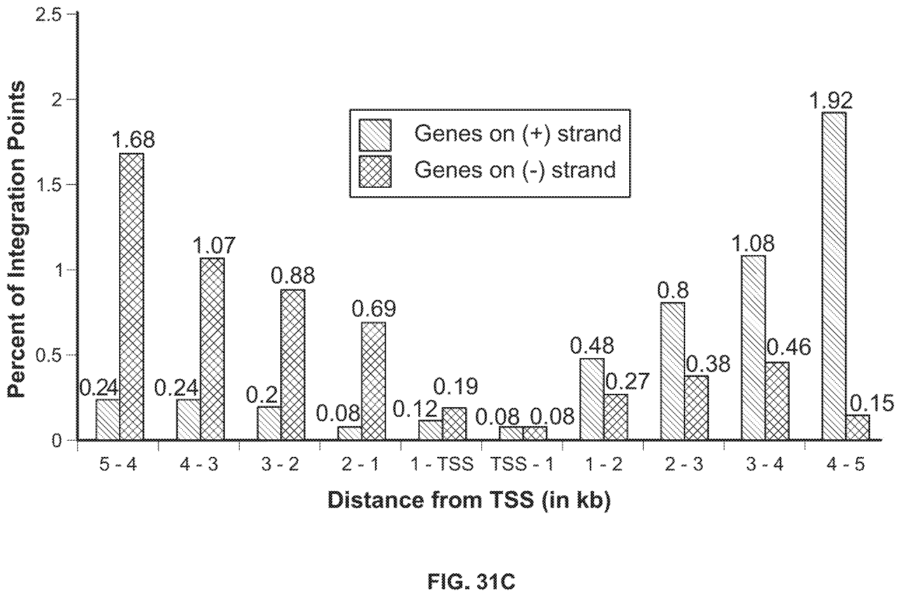

To determine if there is a preference of lentivirus integration in tumorigenic genes, we analyzed integration sties in genic features of all genes in tumor panel containing 754 tumor associated genes (Mayo Large NGS Tumor Panel), and compared with whole genome genes (FIGS. 31A to 31C). Similar pattern of distribution around features was seen in the whole genome and the tumor panel. Statistical analysis shows that integration of lentiviral vector carrying human FAH in tumor associated genes was enriched (p=4.9E-160), but this enrichment was due to the integration in introns (FIG. 31A). The coding regions of tumor associated genes were disfavored by lentivirus (p=0.0003; FIG. 31B). No integration preference in 5 kb upstream of and around transcription start site (TSS) of tumor genes was seen (FIG. 31C). It can be concluded that there was no enrichment of integration in tumor-associated genes with regard to coding regions, 5′- and 3′-UTRs.

### Homology Driven Mechanism May be Involved in the Lentiviral Vector Integration.

In our study, we identified high percentages of lentiviral vector integration events in FAH and SerpinAl genes of human primary hepatocytes which transduced with therapeutic lentiviral vector carrying human FAH coding region and SerpinAl (exon-1) as promoter. Table 5 shows the top 10 genes with the highest amount of coverage.

Further analysis of the integration distribution within FAH gene shows that overwhelming integration events happened in exons. On the other hand, integration distribution in SerpinAl gene shows that integration events located in exon-1 overwhelmingly. We compared each exon size in these two genes with integration frequency, no correlation was found. This interesting discovery implies that homology tethering may be involved in lentiviral integration. To validate this hypothesis, we constructed a new lentiviral vector, using human PAH gene coding region to replace FAH gene in the original lentiviral vector. After transducing human primary hepatocytes, lentiviral integration sites were isolated, sequenced and analyzed in the same as the original vector. The top 10 genes with the highest amount of coverage were shown in Table 5A and B, this time the PAH gene is on the top followed by SerpinAl (Table 5B). Distribution of integration events in PAH gene were investigated and it was found that overwhelming integration events happened in PAH exons only. Very similar results were observed for integration distribution in SerpinAl gene. (Table 5A) These results indicated that homology in lentiviral vectors may play roles in guiding lentivirus integration in host cell genome.

TABLE 5
Top 10 human hepatocytes genes
with highest integration sites
A

| | Number | | | |
|---|---|---|---|---|
| | of | | | |
| | Inte- | Percent of | Number | |
| | gration | Integration | of | Percent of |
| Gene | Points | Points | Reads | Reads |
| FAH | 595 | 0.746390356 | 296399 | 3.100577249 |
| SERPINA1 | 211 | 0.264686238 | 213485 | 2.233228634 |
| CENPC | 41 | 0.05143194 | 57216 | 0.598526405 |
| TBC1D5 | 51 | 0.063976316 | 21271 | 0.22251215 |
| KDM2A | 92 | 0.115408257 | 13305 | 0.13918124 |
| MROH1 | 143 | 0.179384573 | 13160 | 0.137664421 |
| PCDH9 | 98 | 0.122934882 | 12672 | 0.132559539 |
| ROBO1 | 97 | 0.121680445 | 12289 | 0.128553044 |
| CCDC57 | 93 | 0.116662694 | 12023 | 0.125770466 |
| NPLOC4 | 103 | 0.12920707 | 11344 | 0.118667567 |

B

| | | | | |
|---|---|---|---|---|
| PAH | 650 | 0.323003836 | 286595 | 1.473779913 |
| SERPINA1 | 179 | 0.088950287 | 75556 | 0.388537536 |
| PACS1 | 545 | 0.270826294 | 49762 | 0.25589503 |
| CDC428PA | 405 | 0.201256236 | 42975 | 0.220993708 |
| NSD1 | 424 | 0.210697887 | 42485 | 0.218473943 |
| ASH1L | 385 | 0.191317657 | 38010 | 0.1954618 |
| DACH1 | 329 | 0.163489634 | 35330 | 0.181680226 |
| MEIOB | 313 | 0.15553877 | 34154 | 0.175632789 |
| ABCA17P | 300 | 0.149078694 | 29204 | 0.150178016 |
| PPP6R2 | 301 | 0.149575623 | 26593 | 0.136751267 |

### Discussion

In our study, the density of integration sites across human chromosomes revealed a preferential distribution in low- and medium-condensed chromosome regions. These regions are significantly correlated with high gene density in each chromosome. It could be interpreted in terms of chromatin instability as a general effect of lentivirus infection. We compared the relative integration frequency with two different human liver cell gene expression datasets, both of them showed the significantly high integration frequency in highly expressed genes.

In our studies, lentiviral vectors showed highly significant bias in favor of integration in transcript units. The relative integration frequency in exons was enriched higher than in introns (p=1.51E-54), this was due to the contribution of exonic integration events in FAH and SerpinAl genes. When all integration events in these two genes were excluded, the difference between exons and introns was not significant. For integration sites within transcript units, lentivirus did not show a significant bias in quarterly intervals of exons and introns, including 3′- and 5′-UTRs. Within 5 kb upstream of and around TSS, our results showed the disfavored integration. After comparison of lentiviral vectors integration in these genomic features between our results and previous ones on other human cell lines, we noted that the differences were quantitatively minor. This may indicate that cell type is not a crucial factor for lentiviral integration sites selection, perhaps because much of the cellular program of gene regulation and gene activity are shared among many cell types.

CpG islands are the most important feature for evaluating genotoxicity of lentiviral vectors. Our results showed that the relative integration frequency near or in CpG islands was disfavored statistically significant. Thus, for lentiviral vectors, broadly favorable gene-dense chromosomal regions actually contain a mixture of favorable clusters of active genes and unfavorable CpG islands. Base frequency within +50 bp around the integration site was further analyzed. We found that relatively AT-rich regions were characteristic of the target site selection. This observation is consistent with the unfavorable integration in CpG islands.

The ultimate genotoxicity of gene delivery mediated by lentiviral vectors is tumorigenesis. We analyzed the integration sites in 745 tumor associated genes from Mayo Large NGS Tumor Panel. In transcript units, higher integration enrichment was seen in tumor panel genes (p=4.93E-160), this enrichment was due to the higher integration in introns (p=3.59E108). Around and upstream of transcription start sites of tumor panel genes, lentiviral integration did not show any preferences. No increase in the frequency of integration in genomic features in or near tumor genes 5′-end was observed in our study.

A large number of studies on mechanisms of lentivirus integration has provided a wealth of information on the remarkably complex and evolved patterns of interaction between the viral structures and the host genome. Basically, lentiviral integration processes includes, (1) binding to host cell membrane through cell receptors; (2) viral capsid shifts into host cytoplasm and reversely transcribes its RNA to DNA, forms viral pre-integration complex (PIC) with IN at 5′- and 3′-LTRs; (3) nuclear import to host nucleus through nuclear pore; (4) protein mediated tethering of the viral PIC to the host cell chromatin. The PIC is tethered to transcribed gene regions (active gene body), marked by specific histone modifications (H3K20me1, H3K27me1, H3K36me3), by the LEDGF/p75 protein, which interacts with the HIV integrase (IN) through an integrase-binding domain (IBD) and with histones through its PWWP domain. An AT-hook domain (AT) mediates interaction with AT-rich DNA sequences on genomic DNA. In these processes, LEDGF/p75 interacting tightly with viral IN and stimulating the IN catalytic activity is the most important step. In our study, we found an interesting phenomenon that cDNA homology in the construct of lentiviral vector directs lentiviral integration into host homologous region. The possible explanation will be involved in the 4^{th} step described above. While PIC tethers to open chromatin regions, DNA homologous binding process may play a role in this step. Though further experiments need to be done for detailed characterizing this phenomenon, this finding points to the design and development of new lentiviral vector and the nature of the sequences it carries, allowing gene delivery into human genome in a safer way.

### Example 8

### Introduction

To further assess toxicity and specifically address hepatotoxicity/carcinogenicity, we conducted a toxicology study whereby a therapeutic dose of LV-FAH was administered to wild type mice and mice subjected to a chemical liver injury via N-nitrosodiethylamine (DEN) induction and chronic CCl4 exposure. While the wild type mice would generate data to demonstrate inherent toxicity of replication incompetent lentivirus in vivo (if any), the chemical injury model would demonstrate any special toxicity of lentiviral vector exposure/integration in the context of chronic hepatocyte injury and recovery.

### Materials and Methods

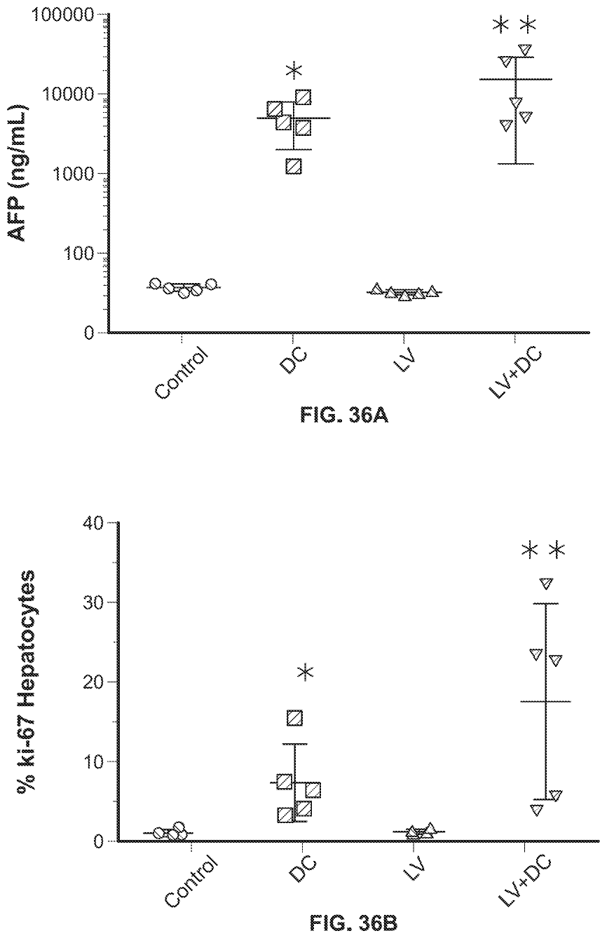

A total of 86 mice were randomized into 1 of 4 groups to receive either vehicle (Groups 1 and 2) or LV-FAH (Groups 3 and 4), with (Groups 2 and 4) or without (Groups 1 and 3) induction of chemical liver injury. Mice were vehicle treated or induced with DEN on Day 1, administered LV-FAH on Day 8/9, and received twice weekly IP injections of CCl4 or olive oil beginning on Day 43 (FIGS. 36A and 36B). Mice were observed for 106 days, at which time all mice were necropsied for post mortem evaluations of toxicity.

We evaluated circulating levels of alpha fetoprotein (AFP) as a biomarker for tumorigenic potential and ki-67 staining as an indicator of hepatocyte proliferation.

### Animals and Animal Care:

All animals received humane care in compliance with the regulations of the institutional animal care and use committee at Mayo Clinic, Rochester, Minn. Mice for the study were bred in house from the laboratory's research colony. Daily observations were performed by animal care/laboratory staff and any clinical concerns were addressed by on-site veterinarians. Body weights were evaluated weekly as part of the toxicological assessment and used as an additional measure of animal health. Animals were dosed intravenously via lateral tail vein (LV-FAH or vehicle) or by intraperitoneal injection (DEN, saline, CCl4, or olive oil). N-nitrosodiethylamine was obtained from Sigma Aldrich (St Louis, Mo.). Carbon tetrachloride was obtained from Acros Organics (Fair Lawn, N.J.). Olive oil was obtained from MP Biomedicals (Solon, Ohio). LV-FAH was formulated in Dulbecco's Modified Eagle Medium (DMEM, ThermoFisher Scientific). Animals were sacrificed by CO2 asphyxiation followed by cervical dislocation consistent with institutional policies for rodent euthanasia. Blood samples were collected post-mortem via the inferior vena cava at necropsy for evaluation of clinical pathology parameters. Tissues were collected immediately after termination, dabbed to remove excess blood, and weighed for calculation of ratios to terminal body weights.

### Lentiviral Vector Construct:

In order to generate the viral vector, a plasmid containing FAH under the control of the alpha-1 antitrypsin promoter was co-transfected with the packaging plasmid p8.91 and the vesicular stomatitis virus glycoprotein G-encoding plasmid pVSV-G into 293T cells using 1 mg/ml polyethylenimine (Polysciences, Warrington, Pa.). Viral supernatant was harvested 48 and 72 h after transfection, filtered through a 0.45-μm filter, and concentrated by ultracentrifugation (25,000 rpm, 1.5 hours at 4° C.). After resuspension in serum-free media (DMEM, Thermo Fisher Scientific, Waltham, Mass.), lentiviral vectors were aliquoted and stored at −80° C. Vector titers were determined by p24 enzyme-linked immunosorbent assay and qPCR using the Lenti-X Provirus Quantitation Kit (Clontech, Mountain View, Calif.). A schematic representation of the lentiviral vector carrying human FAH is provided in FIG. 1.

### Vector Copies Per Genome

Three mice were randomly selected from groups that did not receive LV-FAH (Groups 1 and 2), while 7 animals were randomly selected from groups that did receive LV-FAH (Groups 3 and 4). Liver tissue was recovered from paraffin blocks using Gentra Puregene Tissue Kit (QIAgen, Hilden Germany) per the manufacturer's instructions. Genomic DNA was diluted in Tris-EDTA buffer to 300 ng/μL in 50 μL final volumes for quantitative PCR. A standard curve and duplicates of each liver sample were amplified using SYBR green qPCR with included forward and reverse primers (proprietary to Lenti-X Provirus Quantitation Kit, Takara Bio, Mountain View Calif., formerly and Clontech). Reactions were performed in the ViiA 7 System (Applied Biosystems, Carlsbad, Calif.).

### Biochemical Analysis:

For clinical chemistry analysis, serum was analyzed with the Piccolo® Xpress™ chemistry analyzer (Abaxis, Union City, Calif.) according to the manufacturer's instructions. For hematology analysis, whole blood was analyzed with the VetScan HM5 analyzer (Abaxis, Union City, Calif.) according to the manufacturer's instructions. Alpha-fetoprotein (AFP) was analyzed in serum with the Beckman Coulter Access AFP immunoenzymatic assay on the Beckman Coulter UniCel DXI 800 (Beckman Coulter Inc., Fullerton, Calif.). Tyrosine values were determined using tandem mass spectrometry and chromatography via Mayo Clinic's internal biochemical PKU test.

### Histology Analysis:

For histological analysis, tissue samples were fixed in 10% neutral buffered formalin (Protocol, Fisher-Scientific, and Pittsburgh, Pa.) and processed for paraffin embedding and sectioning. For hematoxylin and eosin staining, slides were prepared with standard protocols and evaluated by a board-certified veterinary pathologist for variations. Ki67 immunohistochemistry was performed using a monoclonal anti-Ki67 primary antibody (MIB-1; Dako/Agilent, Santa Clara, Calif.) as performed with a Bond III automatic stainer (Leica, Buffalo Grove, Ill.) with a 20-min antigen retrieval step using Bond Epitope Retrieval Solution 2 (Leica, Buffalo Grove, Ill.), and stained with diaminobenzidine (Leica, Buffalo Grove, Ill.). Slides used to evaluated fibrosis were stained with Masson's trichrome stain using standard protocols. Ki-67 quantification was performed by selecting 15 random rectangular areas totaling 2.39×10⁷ μm² per slide. Areas were analyzed and quantified using an Aperio ImageScope algorithm that quantifies nuclear staining. Results are reported as percentage of nuclear positivity among cells analyzed.

### Statistical Analysis.

Numerical data are expressed as mean (±standard deviation). Calculations and statistical analysis were performed using Microsoft Excel 2010, and additional statistical analyses were performed with GraphPad Prism software version 7.03. All numerical data were analyzed by 2-tailed student's T test, and differences were considered significant at p<0.05.

### Results

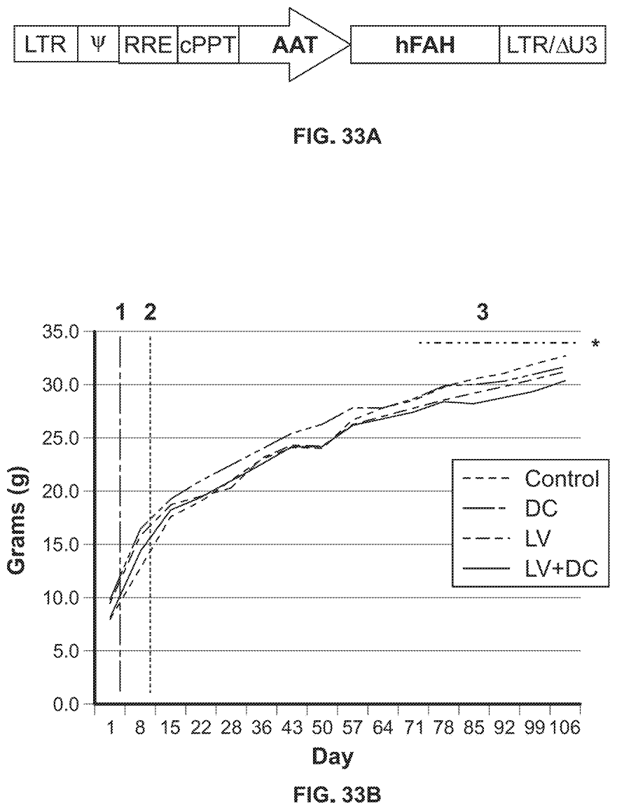

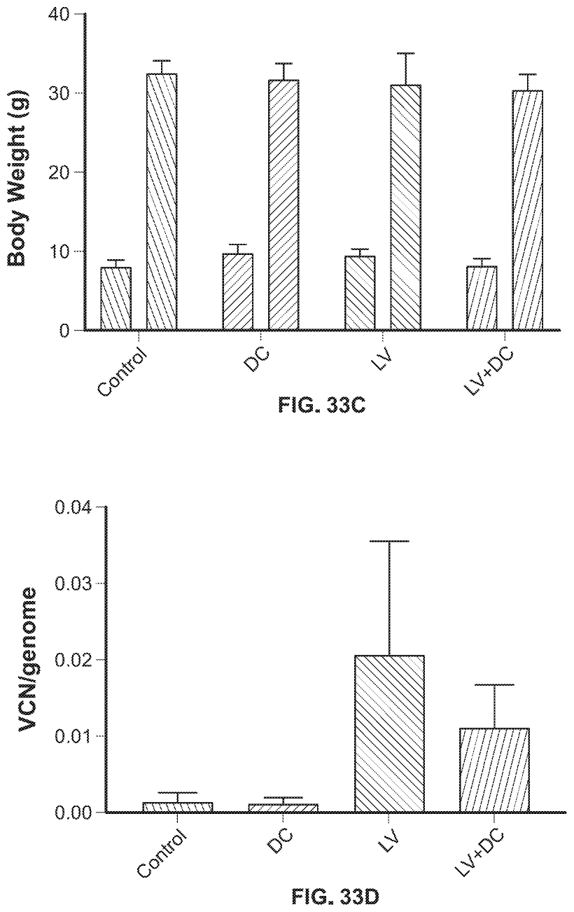

There were 9 early deaths on the study related to complications during dose administration, primarily during the twice-weekly intraperitoneal injection of the CCl4 or empty olive oil vehicle, with no increased incidence from LV-FAH or CCl4 administration (5, 2, 1, and 1 early deaths in Groups 1-4, respectively). There was no measurable exposure to LV-FAH for mice in Groups 1 and 2, while animals in Groups 3 and 4 were positive for 0.021 and 0.011 lentiviral vector copies per mouse genome, respectively, consistent with 1-2% transduced hepatocytes by the end of the observation period (FIG. 33C).

There were no effects of DEN/CCl4, LV-FAH, or their combination on observational data (not shown). Changes in clinical observations were limited to background findings with similar frequency and severity between groups, including control animals. Although animals in all groups gained weight over the course of the study, there was a slight attenuation of total body weight gains in all treated groups relative to the control cohort, whereby control animals gained to 4.2 fold their starting weight of 8.0±1.0 g compared to 3.2, 3.3, and 3.7 fold over starting weights of 9.8±1.1 g, 9.5±0.9 g, and 8.2±0.9 g for DC, LV, and LV+DC groups, respectively (FIG. 33C). This resulted in a slight net decrease in final body weights for the treated groups compared to control animals that was statistically significant in the LV+DC combination-treated group only (Group 4). Although the final difference for all groups was minimal, the DC group and the LV group (Groups 2 and 3) were statistically heavier than the control animals at Day 1, making this decrease by the end of the study possibly more noteworthy.

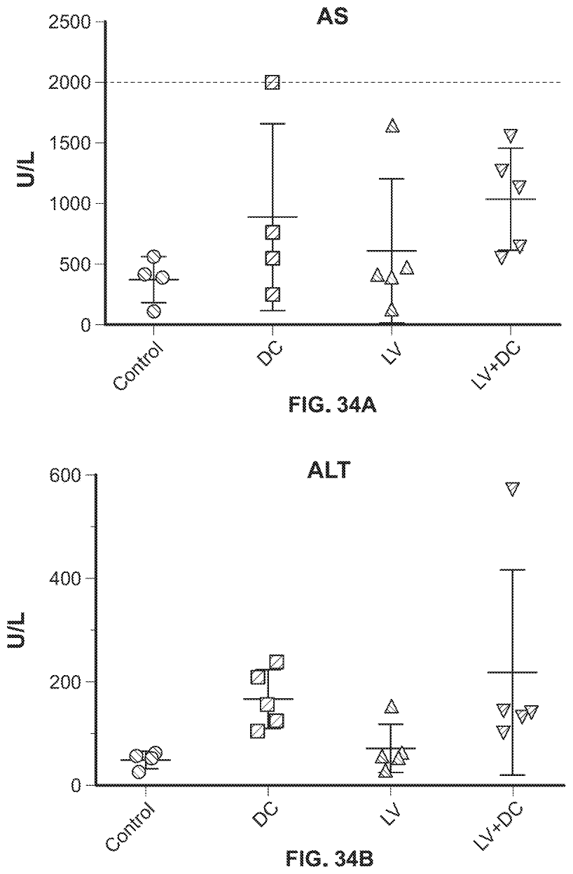

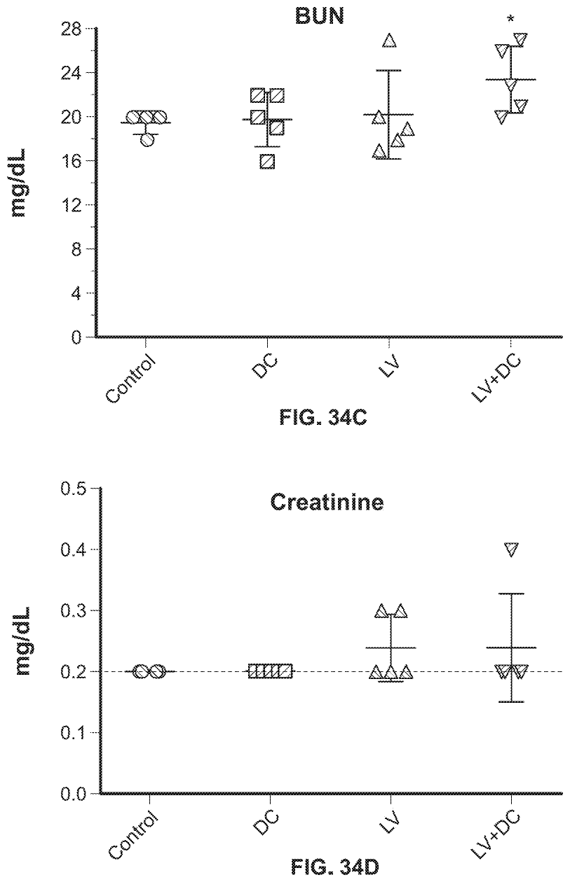

LV-FAH administration alone was not associated with changes in clinical pathology parameters (Group 3). The DEN/CCl4 chemical induction of liver injury (Group 2) was associated with a 3.2× increased in ALT and a 2.4× increase in AST compared to control values (FIGS. 34A and 34B, respectively), and co-administration with LV-FAH trended toward further increases (4.2× and 2.7×, respectively). There was high individual animal variability in these results, and the difference between the treated groups was not significant. There was some hemolysis present in these samples, and it is possible this contributed to volatility in the observed AST levels in individual animals. There was a slight elevation in blood urea nitrogen (BUN) in the combined treatment group only (FIG. 34C) which was present in most animals, and slight elevations in creatinine above the detection threshold in a few animals in both LV-FAH-treated groups (FIG. 34D), but the levels were not toxicologically significant. There were no noteworthy changes in any dose group in hematology parameters tested (Table 6).

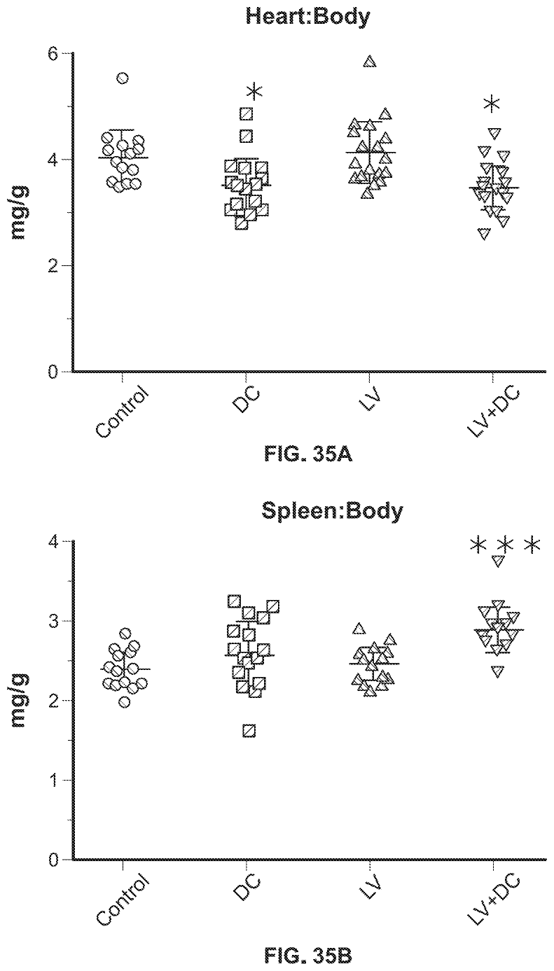

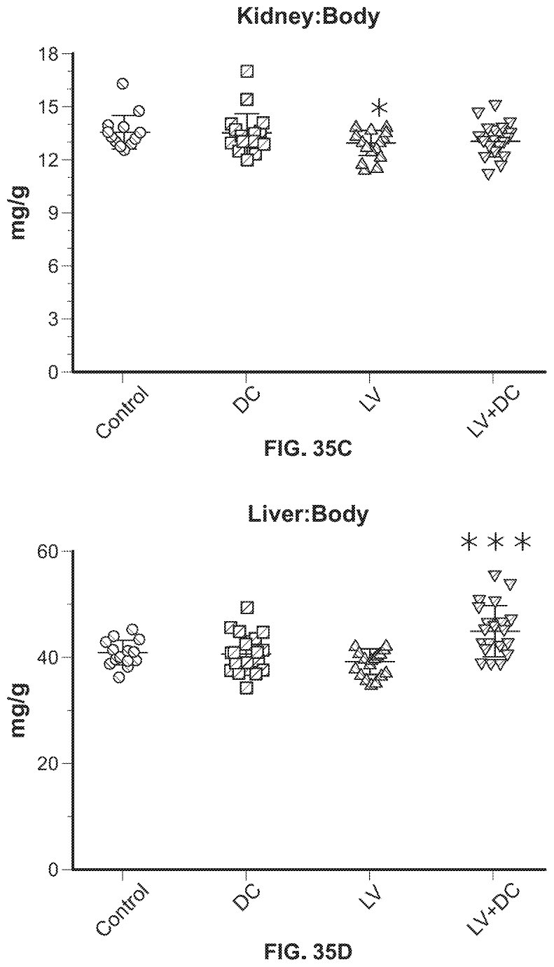

Administration of DEN/CCl4 was associated with slight decreases in heart weights/ratios (FIG. 35A) and increases in spleen weights/ratios (FIG. 35B), regardless of LV-FAH co-administration, although the LV-FAH group trended toward greater increases in the spleen than DEN/CCl4 alone. Administration of LV-FAH was associated with a slight decreases in kidney weights relative to the control group (0.93×, n.s.) which was consistent with the decrease present in the combined treatment group (0.91×, p<0.005), indicating that this slight variation may be associated with LV-FAH administration (FIG. 35C). These decreases were not considered adverse based on the marginal effect in the associated clinical pathology parameters and the absence of gross lesions at necropsy. Interestingly, DEN/CCl4-induced liver injury was not associated with organ weight changes except in combination with LV-FAH, where liver: body weight ratio was 45 mg/g compared to 41 mg/g in control animals (FIG. 35D, p<0.05), contributed to by both a marginal increase in raw liver weights and a significant decrease in body weights. The reduced bodyweight in this group did not appreciably affect ratios of other organs.

DEN/CCl4 was associated with over 100-fold increase in alpha fetoprotein (AFP) compared to control or LV-FAH groups (FIG. 36A, p<0.05 vs. control). Although the difference between the combination treatment and DC alone was an additional 3-fold increase, this trend was the result of exaggerated responses in only 2 of the 5 animals tested and was not significant between the DEN/CCl4 groups. LV-FAH alone had no effect on AFP levels, which would not portend tumor formation as a result of transduction and integration at this level in hepatocytes. LV-FAH alone did not have an effect on baseline ki-67 positivity compared to control animals (FIGS. 36B and C). However, DEN/CCl4 treatment was associated with increased ki-67 positivity of hepatocytes, which wit a trend for greater positivity in the context of LV-FAH co-administration.

### Discussion

There are not many preclinical accounts of toxicity from in vivo lentiviral vector administration. An interesting finding from our study is that the total integration from the single dose was consistent with transduction of 1-2% of the cells by the end of the study. Although this level of integration was of no toxicological consequence in wild type mice, some aspects of the chemical injury model appeared exacerbated by previous exposure to lentiviral vector. Consistent with the findings of Tuppurainen, et. al. (Tuppurainen, et. al. ((2013). Preclinical safety, toxicology, and biodistribution study of adenoviral gene therapy with sVEGFR-2 and sVEGFR-3 combined with chemotherapy for ovarian cancer. Hum Gene Ther Clin Dev 24, 29-37.)

We do not believe this was related to the specific presence or expression of the FAH transgene, but inherent to integration of the LV. The slight trend for less LV in the DEN/CCl4 group by the end of the study might be a product of random variation or sampling accuracy, but it also could be a product of mild selection against the transduced hepatocytes in the regenerative context. Initial transduction rates were not evaluated in this study, but it is possible they were similar, and the chemical injury model resulted in a higher proportion of transduced cells dying off, perhaps due to integration in genes that were important for regenerative capacity.

Lentiviral vectors have shown favorable safety profiles in preclinical and limited clinical studies. Therefore, preclinical studies designed with clinical consideration in vector design, production, and administration are translatable to indicate minimal genotoxicity. There was no evidence of tumorigenicity in any group in this study, which is consistent with our previous ex vivo work with this vector in mouse and pig models of HT1. Furthermore, the use of a liver-specific promoter limits possible transactivation to hepatocytes, where tumorigenesis is already part of the clinical management of human disease via frequent imaging.

The present disclosure is not to be limited in scope by the specific embodiments described herein. Indeed, various modifications of the disclosure in addition to those described herein will become apparent to those skilled in the art from the foregoing description. Such modifications are intended to fall within the scope of the appended claims. All publications referred to herein are hereby incorporated by reference.

What is claimed is:

1. A lentiviral vector comprising an isolated exogenous nucleic acid coding for a functional phenylalanine hydroxylase protein or a functional fumarylacetoacetate hydrolase protein, the isolated exogenous nucleic acid under the control of an alpha1-antitrypsin (AAT) promoter and enhancer operably linked to the isolated exogenous nucleic acid.

2. The lentiviral vector of claim 1, further comprising a 5′ long terminal repeat (LTR) followed by a psi packaging sequence (Ψ) followed by a rev responsive element (RRE) followed by a central polypurine tract (cPPT).

3. The lentiviral vector of claim 2, further comprising a woodchuck hepatitis virus posttranscriptional regulatory element (WPRE) follow by a 3′ LTR having a U3 region that is partially or completely deleted.

4. A method of delivering an isolated exogenous nucleic acid coding for all or part of a polypeptide to a liver of a subject, the method comprising:
administering the isolated exogenous nucleic acid to hepatocytes of the subject intrahepatically or via portal vein injection, the isolated exogenous nucleic acid comprising a nucleic acid coding for all of part of a polypeptide operably linked to a hepatocyte specific promoter and enhancer, wherein the polypeptide is a functional phenylalanine hydroxylase protein or a functional fumarylacetoacetate hydrolase protein.

5. The method of claim 4, wherein the isolated exogenous nucleic acid comprises a lentiviral vector, an adenoviral vector, adeno-associated viral vector or a retroviral vector.

6. The method of claim 4, wherein the isolated exogenous nucleic acid comprises a lentiviral vector.

7. The method of claim 4, wherein the hepatocyte specific promoter is an alpha1-antitrypsin (AAT) promoter.

8. The method of claim 7, wherein the polypeptide is a functional fumarylacetoacetate hydrolase protein.

9. The method of claim 4, wherein the subject is a human.

10. The method of claim 9, wherein the subject is a fetus.

11. The method of claim 9, wherein the subject is an infant or older pediatric patient.

12. The method of claim 4, wherein the isolated exogenous nucleic acid is in a pharmaceutical composition.

13. The method of claim 4, wherein the isolated exogenous nucleic acid is contained within a hepatocyte of the same species as the subject.

14. The method of claim 4, wherein the isolated exogenous nucleic acid is contained within a stem cell comprising an adult hepatic stem cell, a fetal hepatic stem cell, or an embryonic stem cell.

15. The method of claim 4, wherein the isolated exogenous nucleic acid is contained within a cell obtained or derived from the subject.

16. A human hepatocyte comprising a lentiviral vector comprising an isolated exogenous nucleic acid coding for all or part of a polypeptide under the control of a hepatocyte specific promoter and enhancer linked to all or part of the isolated nucleic acid, the polypeptide comprising a functional fumarylacetoacetate hydrolase, a functional tyrosine transaminase, a functional 4-hydroxy-phenylpyruvate dioxygenase, or a functional phenylalanine hydroxylase.

17. The human hepatocyte of claim 16, wherein the hepatospecific promoter comprises a spleen focus forming virus promoter, a cytomegalovirus promoter, or an alpha1-antitrypsin (AAT) promoter.

18. The human hepatocyte of claim 16, wherein the hepatospecific promoter comprises a human alpha1-antitrypsin (AAT) promoter.

19. The human hepatocyte of claim 16, wherein the polypeptide comprises a functional functional phenylalanine hydroxylase.

20. The human hepatocyte of claim 16, wherein the polypeptide comprises a functional fumarylacetoacetate hydrolase.
